(19) United States
(12) Patent Application Publication
(10) Pub. No.: US 2025/0029689 A1
(43) Pub. Date: Jan. 23, 2025

(54) SYSTEMS AND METHODS FOR SPACE HEALTH EXPOSOMICS

(71) Applicant: Linus Biotechnology Inc., North Brunswick, NJ (US)

(72) Inventors: Manish ARORA, New Rochelle, NY (US); Paul CURTIN, Jersey City, NJ (US); Christine AUSTIN, New York, NY (US)

(21) Appl. No.: 18/906,598

(22) Filed: Oct. 4, 2024

Related U.S. Application Data
(63) Continuation of application No. PCT/US2023/017676, filed on Apr. 6, 2023.
(60) Provisional application No. 63/328,164, filed on Apr. 6, 2022.

(51) Int. Cl. G16H 10/20; G16H 10/40; G16H 50/20

(52) U.S. Cl. CPC G16H 10/20; G16H 10/40; G16H 50/20

(57) ABSTRACT

Provided herein are methods and systems configured to analyze exposomic features of subjects undergoing space travel.

### CROSS-REFERENCE

This application is a by-pass continuation application of International Application No. PCT/US2023/017676, filed Apr. 6, 2023, which claims the benefit of U.S. Provisional Patent Application No. 63/328,164 filed Apr. 6, 2022, each of which are incorporated herein by reference in their entirety.

### BACKGROUND

The immediate and lasting effects of space travel on the mammals (including humans) is unknown. With the dawn of the space exploration era, developing a deep understanding of the possible effects of space travel and how to ameliorate negative effects imposed onto individuals is necessary. Therefore, there is an unmet need for systems and methods configured to screen individuals participating in space travel minimally invasively to determine the resulting effect of space travel and what interventions may ameliorate the negative effects of space travel.

### SUMMARY

Besides the apparent macro physiological and/or anatomical effects endured by prolong space travel, the effects of space travel and/or transient space travel on the human body are not well understood. To sustain prolonged space travel and/or frequent exposure to space travel, there exists an unmet need of a platform capable of measuring the influence of space or simulated space travel on human physiology and/or anatomy. The disclosure provided herein, in some cases, may describe methods and systems for analyzing subjects' exposomic signatures that are affected by space travel and/or simulated space travel. In some cases, the disclosure provided herein, describes methods and systems for determining the effect of an intervention and/or diet on one or more exposomic signature and/or exposomic features of subjects that may be used to counter act the effects space travel has on the one or more exposomic signatures and/or exposomic features of subjects.

The present disclosure addresses these needs through systems and methods capable of analyzing and classifying subjects by their exposomic biochemical signatures. Exposomic biochemical signature analysis is capable of analyzing over 50,000 biochemical signatures in a non-invasive manner from a single hair shaft, tooth, and nail sample. With such systems and methods of analysis of the present disclosure, subtle changes in a subject's biochemistry induced by diet, air-pollution, space travel, psychological stress, exposure to pesticides, industrial chemicals, etc. may be investigated and correlated to positive response and outcomes of targeted pharmaceuticals.

In addition to the copious dataset generated by exposomic biochemical signature analysis, exposomic biochemical signature analysis can also provide insight into temporal fluctuations of said biochemical signatures over the span of a subject's life. Such an approach may be utilized to screen individuals participating in space travel and/or simulated space travel to determine what single or combination of exposomic biochemical signatures are affected significantly from space travel and/or simulated space travel. The identified pathways may then be used to train statistical, machine learning, and/or artificial intelligence predictive models capable of predicting whether or not a subject's exposomic signatures and/or features are associated with space travel.

Aspects of the disclosure disclosed herein provide a computer-implemented exposomics system, the system comprising: (a) an exposome biochemical signatures database (EDB) comprising exposomic features for a plurality of subjects that have and have not participated in space travel; (b) a clinical database (CDB) comprising clinical phenotype information for the plurality of subjects participating in space travel; (c) an intervention outcome database (IODB) comprising information on intervention outcome information for the subjects that have received an intervention after, during, and/or prior to participating in space travel; and (d) a computer processor comprising: (i) an association software module communicatively coupled to the EDB, the CDB, and the IODB, where the association software module is programmed to determine an association between the exposomic features, the clinical phenotype information, and the intervention outcome information for at least one of the plurality of subjects, and (ii) a recommendation software module communicatively coupled to the EDB, the CDB, and the IODB. The recommendation software module is programmed to provide an intervention recommendation for the at least one of the plurality of subjects based at least in part on the exposomic features, the clinical phenotype information, the intervention outcome information, and the association between the exposomic features, the clinical phenotype information, and the intervention outcome information for the at least one of the plurality of subjects.

In some embodiments, the exposomic features comprises at least 10, at least 100, at least 1,000, or at least 10,000 distinct exposomic biochemical signatures. In some embodiments, the intervention outcome information comprises classifications of non-responder, adverse responder, and positive responder for at least one intervention ameliorating the negative effects of space travel. In some embodiments, the intervention outcome comprises one or more inclusion criteria or exclusion criteria for at least one intervention. In some embodiments, the exposomic features is obtained by assaying biological samples of the plurality of subjects. In some embodiments, the biological samples comprise tooth samples, nail samples, hair samples, or any combination thereof. In some embodiments, the assaying comprises obtaining mass spectrometry measurements, laser induced breakdown spectroscopy measurements, laser ablation-inductively coupled plasma mass spectrometry measurements, Raman spectroscopy measurements, breakdown spectroscopy, immunohistochemistry measurements, or any combination thereof. In some embodiments, the mass spectrometry measurements comprise measurements of one or more chemicals. In some embodiments, the one or more chemicals comprise aluminum, arsenic, barium, bismuth, calcium, copper, iodide, lead, lithium, magnesium, manganese, phosphorus, sulfur, tin, strontium, zinc, or any combination thereof chemicals. In some embodiments, the exposomic features comprises dynamic temporal biochemical responses of the plurality of subjects. In some embodiments, the exposome biochemical signatures comprises fluorescence images of the biological samples. In some embodiments, the exposome biochemical signatures comprises spatial maps of Raman spectra of the biological samples. In some embodiments, the exposome biochemical signatures are associated with space travel and/or simulated space travel. In some embodiments, the exposomic features are analyzed using a trained statistical, machine learning, and/or artificial intelligence classifier to determine the association with the space travel. In some embodiments, the classifier is selected from the group consisting of: a neural network algorithm, a support vector machine algorithm, a decision tree algorithm, an unsupervised clustering algorithm, a supervised clustering algorithm, a regression algorithm, a gradient-boosting algorithm, and any combination thereof.

Aspects of the disclosure disclosed herein describe a method for selecting a subject participating in space travel for an intervention, the method comprising: (a) providing a trained predictive model, where the trained predictive model is trained on one or more subjects' clinical metadata, exposomic features, and corresponding intervention outcome information; (b) detecting a biochemical signature obtained from a biological sample from a subject seeking the intervention, thereby producing exposomic features; (c) predicting, with the trained predictive model, the predicted intervention outcome information of the subject seeking the intervention, where the exposomic features and clinical meta of the subject seeking the intervention are inputs to the trained predictive model; and (d) selecting the subject for the intervention or excluding the subject from the intervention, based at least in part on the predicted intervention outcome information of the subject.

In some embodiments, the biochemical signature is obtained by assaying a biological sample of the subject. In some embodiments, the biological sample comprises a tooth sample, a nail sample, a hair sample, or any combination thereof. In some embodiments, the assaying comprises collecting data from laser ablation-inductively coupled plasma mass spectrometry measurements, laser induced breakdown spectroscopy measurements, Raman spectroscopy measurements, immunohistochemistry measurements, or any combination thereof. In some embodiments, the laser ablation-inductively coupled plasma mass spectrometry measurements comprise measurements of one or more chemicals. In some embodiments, the one or more chemicals comprise aluminum, arsenic, barium, bismuth, calcium, copper, iodide, lead, lithium, magnesium, manganese, phosphorus, sulfur, tin, strontium, zinc, or any combination thereof. In some embodiments, the biochemical signature comprises fluorescence images of the biological sample. In some embodiments, the biochemical signature comprises spatial maps of Raman spectra of the biological sample. In some embodiments, the biochemical signature is associated with a space travel and/or simulated space travel. In some embodiments, the trained predictive model is selected from the group consisting of: a neural network algorithm, a support vector machine algorithm, a decision tree algorithm, an unsupervised clustering algorithm, a supervised clustering algorithm, a regression algorithm, a gradient-boosting algorithm, and any combination thereof. In some embodiments, the method further comprises enrolling the subject into the intervention, when the subject is selected for the intervention. In some embodiments, the method further comprises evaluating the subject for another intervention, when the subject is excluded from the intervention.

Aspects of the disclosure describe a method of selecting an optimal treatment for a subject participating in space travel and/or simulated space travel, comprising: (a) detecting one or more biochemical signatures obtained from one or more biological samples from one or more subjects that have not participating in space travel, thereby producing one or more reference exposomic features; (b) detecting one or more biochemical signature obtained from one or more biological samples from subject that have participated in space travel, thereby producing one or more pre-treatment exposomic features; (c) administering a treatment subjects that have participated in space travel; (d) detecting one or more biochemical signatures obtained from one or more biological samples from one or more subjects that have participated in space travel after a period of time has elapsed after receiving the treatment, thereby producing one or more post-treatment exposomic features; (e) determining a difference between the one or more reference exposomic features of the one or more subjects that have not participated in space travel, the one or more pre-treatment exposomic features of the one or more subjects participating in space travel, and the one or more post-treatment exposomic features of the one or more subjects that have participated in space travel; and (f) selecting one or more optimal treatments based at least in part on the determined difference between the one or more reference exposomic features, one or more pre-treatment exposomic features, and one or more post-treatment exposomic features, where the one or more optimal treatments are selected based on the determined differences satisfying a pre-determined criterion. In some embodiments, the optimal treatment may comprise a pharmaceutical, nutraceutical, or any combination thereof. In some embodiments, the pre-determined criterion comprises a difference between the one or more pre-treatment exposomic features and the one or more post-treatment exposomic features to the one or more reference exposomic features. In some embodiments, the period of time comprises at least about 1 hour, at least about 1 day, at least about 1 week, at least about 1 month, at least about 1 year, or any combination thereof. In some embodiments, the difference comprises a change of at least 10% of the one or more post-treatment exposomic features toward the one or more reference exposomic features. In some embodiments, the pre-treatment exposomic features, post-treatment exposomic features, or any combination thereof is obtained by assaying a biological sample of the subject. In some embodiments, the biological sample comprises a tooth sample, a nail sample, a hair sample, or any combination thereof. In some embodiments, the assaying comprises obtaining laser ablation-inductively coupled plasma mass spectrometry data, laser induced breakdown spectroscopy measurements, Raman spectroscopy measurements, immunohistochemistry measurements, or any combination thereof. In some embodiments, the laser ablation-inductively coupled plasma mass spectrometry data comprises measurements of one or more chemicals. In some embodiments, the one or more chemicals comprise aluminum, arsenic, barium, bismuth, calcium, copper, iodide, lead, lithium, magnesium, manganese, phosphorus, sulfur, tin, strontium, zinc, or any combination thereof chemicals. In some embodiments, the biochemical signature comprises fluorescence images of the biological sample. In some embodiments, the biochemical signature comprises spatial maps of Raman spectra of the biological sample. In some embodiments, the differences between the reference exposomic features, pre space travel exposomic features, post space travel exposomic features, or any combination thereof, is analyzed using a trained statistical, machine learning, and/or artificial intelligence classifiers. In some embodiments, the trained classifier is selected from the group consisting of: a neural network algorithm, a support vector machine algorithm, a decision tree algorithm, an unsupervised clustering algorithm, a supervised clustering algorithm, a regression algorithm, a gradient-boosting algorithm, and any combination thereof.

The present disclosure provides improved systems and methods for accurate determination of the effect of space travel and/or simulated space travel based on analysis of dynamic biological response data from non-invasively obtained biological samples from subjects. Such improved systems and methods for accurate determination of the one or more exposomic signatures and/or exposomic features may be based on a combination of dynamic immunohistochemistry profiling of biological samples and artificial intelligence data analysis of such dynamic profiles toward assessment of how space travel influences exposomic signatures and/or features. The present disclosure addresses these needs, for example, by providing a biological sample biomarker for analyzing the one or more exposomic signatures and/or features associated with space travel. The biological sample includes a human biological specimen that is associated with incremental growth. In nonlimiting embodiments, the biological sample is a hair shaft, a tooth, a toenail, a fingernail, a physiologic parameter, or any combination thereof. The non-invasive biomarker of the present disclosure can be used for the diagnosis of young children, even infants younger than one year old. In some embodiments, the f comprises health meta data described elsewhere herein. In some embodiments, the analysis of the one or more exposomic signatures and/or exposomic features influence by space travel and/or simulated space travel may comprise analyzing one or more physiologic parameter comprising a parameter measured during a blood test, e.g., cholesterol, white blood cell count, red blood cell count, hematocrit, the presence, or lack thereof bacterial infection, etc.

In some embodiments, the analyzing determines temporal dynamics of underlying biological processes. In some embodiments, the analyzing comprises obtaining a fluorescence image of the stained tooth sample and analyzing the fluorescence intensity of the fluorescence image. In some embodiments, the fluorescence intensity is spatially varying. In some embodiments, obtaining the fluorescence image of the stained tooth sample comprises using an inverted or non-inverted confocal microscope. In some embodiments, staining the tooth sample comprises using a C-reactive protein immunohistochemistry stain. In some embodiments, the method further comprises sectioning the tooth sample. In some embodiments, staining the tooth sample comprises (1) cutting the tooth sample, (2) decalcifying the tooth sample, (3) sectioning the decalcified sample, (4) staining decalcified tooth sections with primary and secondary antibodies, (5) measuring the spatial antibody fluorescence with confocal microscopy, and/or (6) extracting a temporal profile of fluorescence intensity.

In an aspect, the present disclosure provides a method for outputting one or more subjects' response to participating in space travel, comprising: receiving one or more biological samples of one or more subjects participating in space travel; determining one or more exposomic signatures of the one or more biological samples; calculating exposomic features of the one or more biological samples' one or more exposomic signatures; and outputting the one or more subjects' response to participating in space travel by providing the exposomic features of the one or more biological samples, or by utilizing the exposomic features of the one or more biological samples as an input into a trained predictive model, which provides a binary indicator of the biological response to space travel. In some embodiments, the trained predictive model is configured to determine a likelihood that the exposomic features are associated with metabolic signatures of one or more subjects participating in space travel. In some embodiments, space travel comprises traveling beyond the Karman line at an altitude of at least 100 kilometers (km) above the Earth's surface. In some embodiments, the trained predictive model is configured to classify if or more subjects' samples are consistent or not consistent with real or simulated space travel. In some embodiments, the one or more biological samples of the subjects participating in the space travel is collected prior to the space travel, during the space travel, after the space travel, or any combination thereof periods of the space travel. In some embodiments, the biological sample comprises hair, teeth, toenails, fingernails, physiologic parameters, or any combination thereof.

In some embodiments, each feature of the exposomic features comprises one or more quantitative metrics. In some embodiments, the exposomic features comprise a measurement of temporal dynamics of the one or more exposomic signatures. In some embodiments, the measurement of the temporal dynamics comprises: determination of a linear slope, determination of a plurality of non-linear parameters describing curvature of the one or more exposomic signatures, determination of an abrupt change in intensity of the one or more exposomic signatures, determination of one or more changes in a baseline intensity of the one or more exposomic signatures, determination of a change of a frequency-domain representation of the one or more exposomic signatures, determination of a change of the power-spectral domain representation of the one or more exposomic signatures, determination of one or more recurrence quantification analysis parameters, determination of one or more cross-recurrence quantification analysis parameters, determination of one or more joint recurrence quantification analysis parameters, determination of one or more multidimensional recurrence quantification analysis parameters, estimation of a Lyapunov spectra, determination of a maximum Lyapunov exponent, or any combination thereof. In some embodiments, the measurement of the temporal dynamics comprises determination of one or more recurrence quantification analysis parameters, wherein the one or more recurrence quantification analysis parameters comprises a recurrence rate, a determinism, a mean diagonal length, a maximum diagonal length, a divergence, a Shannon entropy in diagonal length, a trend in recurrence, a laminarity, a trapping time, a maximum vertical line length, a Shannon entropy in vertical line length, a mean recurrence time, a Shannon entropy in recurrence time, or a number of a most probable recurrence.

In some embodiments, the one or more exposomic signatures are measured by mass spectrometry, laser ablation-inductively coupled plasma-mass spectrometry, laser induced breakdown spectroscopy, Raman spectroscopy, immunohistochemistry fluorescence, or any combination thereof. In some embodiments, the exposomic features are derived from analysis of one or more attractors. In some embodiments, the method further comprises analyzing a dynamic relationship between the one or more attractors' signal recurrence rates, determinism, mean diagonal length, maximum diagonal length, divergence, Shannon entropy in diagonal length, trend in recurrences, laminarity, trapping time, maximum vertical line length, Shannon entropy in vertical line lengths, mean recurrence time, Shannon entropy in recurrence times, number of the most probable recurrences, or any combination thereof.

In some embodiments, the one or more subjects are a human, non-human mammal, vertebrate, or any combination thereof.

In some embodiments, the trained predictive model comprises a classification algorithm. In some embodiments, the classification algorithm is configured to identify the one or more exposomic signatures of the one or more subjects associated with participating in space travel. In some embodiments, the classification algorithm is trained with the one or more subjects' one or more biological samples exposomic features and corresponding responses, wherein the one or more subjects participated in space travel. In some embodiments, the classification algorithm is configured to determine if the one or more subjects have a metabolic signature associated with space travel.

In some embodiments, the response comprises a state of the subject's metabolism, wherein the subject's metabolism comprises a healthy state or a state consistent with a subject undergoing space-travel.

In an aspect, the present disclosure provides a method for training a predictive model to determine a clinical response of subjects to an intervention when the subject is participating in space travel, comprising: receiving one or more biological samples and clinical responses of a first set of subjects, wherein the first set of subjects are exposed to space travel and are provided an intervention, and a second set of subjects, wherein the second set of subjects are exposed to space travel and do not receive the intervention; determining one or more exposomic signatures of the one or more biological samples of the first and second set of subjects; calculating a first and second set of exposomic features of the first and second set of subjects' one or more exposomic signatures, wherein each feature of the first and second set of exposomic features comprises one or more quantitative metrics; and outputting a trained predictive model configured to determine a clinical response of a subject to an intervention, wherein the subject is participating in space travel, and wherein the trained predictive model is trained on the first and second set of subjects' one or more exposomic features' one or more quantitative metrics and the corresponding clinical responses.

In some embodiments, the intervention prevents negative side effects of space travel. In some embodiments, the intervention is provided to the first set of subjects prior to, during, after, or any combination thereof time point of space travel the subjects have been exposed to.

In some embodiments, the method further comprises determining an influence of space travel on the first set of subjects exposed to the intervention at least in part by comparing the first and second set of exposomic features' one or more quantitative metrics. In some embodiments, the intervention comprises a diet, pharmaceutical therapeutic, or any combination thereof. In some embodiments, the diet comprises iodine supplementation.

In some embodiments, the first or second set of exposomic features comprise a measurement of temporal dynamics of the one or more exposomic signatures. In some embodiments, the measurement of the temporal dynamics comprises: determination of a linear slope, determination of a plurality of non-linear parameters describing curvature of the one or more exposomic signatures, determination of an abrupt change in intensity of the one or more exposomic signatures, determination of one or more changes in a baseline intensity of the one or more exposomic signatures, determination of a change of a frequency-domain representation of the one or more exposomic signatures, determination of a change of the power-spectral domain representation of the one or more exposomic signatures, determination of one or more recurrence quantification analysis parameters, determination of one or more cross-recurrence quantification analysis parameters, determination of one or more joint recurrence quantification analysis parameters, determination of one or more multidimensional recurrence quantification analysis parameters, estimation of a Lyapunov spectra, determination of a maximum Lyapunov exponent, or any combination thereof. In some embodiments, the measurement of the temporal dynamics comprises determination of one or more the recurrence quantification analysis parameters, wherein the one or more the recurrence quantification analysis parameters comprises a recurrence rate, a determinism, a mean diagonal length, a maximum diagonal length, a divergence, a Shannon entropy in diagonal length, a trend in recurrence, a laminarity, a trapping time, a maximum vertical line length, a Shannon entropy in vertical line length, a mean recurrence time, a Shannon entropy in recurrence time, or a number of a most probable recurrence.

In some embodiments, the one or more exposomic signatures are measured by mass spectrometry, laser ablation-inductively coupled plasma-mass spectrometry, laser induced breakdown spectroscopy, Raman spectroscopy, immunohistochemistry fluorescence, or any combination thereof.

In some embodiments, the one or more biological samples comprise hair, teeth, toenails, fingernails, physiologic parameters, or any combination thereof.

In some embodiments, the first or second set of exposomic features are derived from one or more attractors. In some embodiments, the method further comprises analyzing a dynamic relationship between the one or more attractors' signal recurrence rates, determinism, mean diagonal length, maximum diagonal length, divergence, Shannon entropy in diagonal length, trend in recurrences, laminarity, trapping time, maximum vertical line length, Shannon entropy in vertical line lengths, mean recurrence time, Shannon entropy in recurrence times, number of the most probable recurrences, or any combination thereof.

In some embodiments, the first or second set of subjects are human, non-human mammals, or any combination thereof.

In an aspect, the present disclosure provides a method for generating a predictive model configured to output one or more subjects' health state in response to a gravitational force in addition to the earth's gravitational force, comprising: receiving a first one or more subjects' one or more biological samples and corresponding health states, wherein the first one or more subjects' have received an exposure to a gravitational force in addition to the earth's gravitational force; determining one or more exposomic signatures of the first one or more subjects' one or more biological samples; calculating exposomic features of the one or more biological samples' one or more exposomic signatures; and generating a trained predictive mode configured to output a second one or more subjects' health state in response to a gravitational force in addition to the earth's gravitational force, wherein the trained predictive model is trained with the first one or more subjects' exposomic features of the one or more exposomic signatures and corresponding health states. In some embodiments, the one or more biological samples comprise hair, teeth, toenails, fingernails, physiologic parameters, or any combination thereof.

In some embodiments, the gravitational force comprises at least 1 G, at least 2 G, at least 3G, or at least 4G.

In some embodiments, the trained predictive model is configured to determine a likelihood that the exposomic features are associated with metabolic signatures of the first or second one or more subject exposed to a gravitational force in addition to the earth's gravitational force.

In some embodiments, a probabilistic threshold determined by the trained predictive model indicates that the first or second one or more subjects' metabolism is at baseline conditions not associated with an exposure to a gravitational force in addition to the earth's gravitational force.

In some embodiments, each feature of the exposomic features comprises one or more quantitative metrics. In some embodiments, the exposomic features comprise a measurement of temporal dynamics of the one or more exposomic signatures. In some embodiments, the measurement of the temporal dynamics comprises: determination of a linear slope, determination of a plurality of non-linear parameters describing curvature of the one or more exposomic signatures, determination of an abrupt change in intensity of the one or more exposomic signatures, determination of one or more changes in a baseline intensity of the one or more exposomic signatures, determination of a change of a frequency-domain representation of the one or more exposomic signatures, determination of a change of the power-spectral domain representation of the one or more exposomic signatures, determination of one or more recurrence quantification analysis parameters, determination of one or more cross-recurrence quantification analysis parameters, determination of one or more joint recurrence quantification analysis parameters, determination of one or more multidimensional recurrence quantification analysis parameters, estimation of a Lyapunov spectra, determination of a maximum Lyapunov exponent, or any combination thereof. In some embodiments, the measurement of the temporal dynamics comprises determination of one or more recurrence quantification analysis parameters, wherein the one or more recurrence quantification analysis parameters comprises a recurrence rate, a determinism, a mean diagonal length, a maximum diagonal length, a divergence, a Shannon entropy in diagonal length, a trend in recurrence, a laminarity, a trapping time, a maximum vertical line length, a Shannon entropy in vertical line length, a mean recurrence time, a Shannon entropy in recurrence time, or a number of a most probable recurrence.

In some embodiments, the one or more exposomic signatures are measured by mass spectrometry, laser ablation-inductively coupled plasma-mass spectrometry, laser induced breakdown spectroscopy, Raman spectroscopy, immunohistochemistry fluorescence, or any combination thereof.

In some embodiments, the exposomic features are derived from one or more attractors.

In some embodiments, the method further comprises analyzing a dynamic relationship between the one or more attractors' signal recurrence rates, determinism, mean diagonal length, maximum diagonal length, divergence, Shannon entropy in diagonal length, trend in recurrences, laminarity, trapping time, maximum vertical line length, Shannon entropy in vertical line lengths, mean recurrence time, Shannon entropy in recurrence times, number of the most probable recurrences, or any combination thereof.

In some embodiments, the first and second one or more subjects are human, non-human mammal, vertebrate, or any combination thereof.

In some embodiments, the trained predictive model comprises a classification algorithm.

In some embodiments, the classification algorithm is configured to identify the one or more exposomic signatures of the first or second one or more subject associated with exposure to the gravitational force in addition to the earth's gravitational force.

In some embodiments, the classification algorithm is trained with the first one or more subjects' one or more biological samples' exposomic features and corresponding health states, wherein the first one or more one or more subjects were exposed to the gravitational force in addition to the earth's gravitational force.

In some embodiments, the classification algorithm is configured to determine if the first or second one or more subjects' have metabolic signatures associated with exposure to the gravitational force in addition to the earth's gravitational force.

In some embodiments, the health state comprises a state of the first or second one or more subjects' metabolism, wherein the first or second one or more subjects' metabolism comprises a healthy state or a state consistent with a subject exposed to the gravitational force in addition to the earth's gravitational force.

In an aspect, the present disclosure provides a method for using a predictive model to output a health outcome of one or more subjects prior to exposure of a gravitational force, comprising: receiving one or more subjects' one or more biological samples; determining one or more exposomic signatures of the one or more subjects' one or more biological samples; calculating exposomic features of the one or more biological samples' one or more exposomic signatures; and outputting a health outcome of the one or more subjects prior to exposure of a gravitational force in addition to the gravitational force of the earth by providing the one or more subjects' exposomic features as an input to a trained predictive model.

In some embodiments, the gravitational force comprises at least 1 G, at least 2 G, at least 3G, or at least 4G.

In some embodiments, the trained predictive model is configured to determine a likelihood that the exposomic features are associated with metabolic signatures of the one or more subjects exposed to the gravitational force.

In some embodiments, a probabilistic threshold determined by the trained predictive model indicates that one or more subjects' metabolism is at baseline conditions not associated with the exposure to the gravitational force in addition to the earth's gravitational force.

In some embodiments, each feature of the exposomic features comprises one or more quantitative metrics.

In some embodiments, the one or more biological samples comprise hair, teeth, toenails, fingernails, physiologic parameters, or any combination thereof.

In some embodiments, the exposomic features comprise a measurement of temporal dynamics of the one or more exposomic signatures.

In some embodiments, the measurement of the temporal dynamics comprises: determination of a linear slope, determination of a plurality of non-linear parameters describing curvature of the one or more exposomic signatures, determination of an abrupt change in intensity of the one or more exposomic signatures, determination of one or more changes in a baseline intensity of the one or more exposomic signatures, determination of a change of a frequency-domain representation of the one or more exposomic signatures, determination of a change of the power-spectral domain representation of the one or more exposomic signatures, determination of one or more recurrence quantification analysis parameters, determination of one or more cross-recurrence quantification analysis parameters, determination of one or more joint recurrence quantification analysis parameters, determination of one or more multidimensional recurrence quantification analysis parameters, estimation of a Lyapunov spectra, determination of a maximum Lyapunov exponent, or any combination thereof.

In some embodiments, the measurement of the temporal dynamics comprises determination of one or more recurrence quantification analysis parameters, wherein the one or more recurrence quantification analysis parameters comprises a recurrence rate, a determinism, a mean diagonal length, a maximum diagonal length, a divergence, a Shannon entropy in diagonal length, a trend in recurrence, a laminarity, a trapping time, a maximum vertical line length, a Shannon entropy in vertical line length, a mean recurrence time, a Shannon entropy in recurrence time, or a number of a most probable recurrence.

In some embodiments, the one or more exposomic signatures are measured by mass spectrometry, laser ablation-inductively coupled plasma-mass spectrometry, laser induced breakdown spectroscopy, Raman spectroscopy, immunohistochemistry fluorescence, or any combination thereof.

In some embodiments, the exposomic features are derived from one or more attractors.

In some embodiments, the method further comprises analyzing a dynamic relationship between the one or more attractors' signal recurrence rates, determinism, mean diagonal length, maximum diagonal length, divergence, Shannon entropy in diagonal length, trend in recurrences, laminarity, trapping time, maximum vertical line length, Shannon entropy in vertical line lengths, mean recurrence time, Shannon entropy in recurrence times, number of the most probable recurrences, or any combination thereof.

In some embodiments, the one or more subjects comprise a human, non-human mammal, vertebrate, or any combination thereof.

In some embodiments, the trained predictive model comprises a classification algorithm.

In some embodiments, the classification algorithm is configured to identify the one or more exposomic signatures of the subject associated with an exposure to the gravitational force in addition to the earth's gravitational force.

In some embodiments, the classification algorithm is trained with one or more subjects' one or more biological samples' exposomic features and corresponding responses, wherein the one or more subjects are exposed to a gravitational force in addition to the earth's gravitational force.

In some embodiments, the classification algorithm is configured to determine if the one or more subjects' have metabolic signatures associated with exposure to a gravitational force in addition to the earth's gravitational force.

In an aspect, the present disclosure provides a method for administering a treatment to a subject participating in space travel, comprising: receiving one or more biological samples of a subject prior to participating in space travel; determining one or more exposomic signatures of the one or more biological samples of the subject; calculating exposomic features of the subject's one or more exposomic signatures, wherein each feature of the exposomic features comprises one or more quantitative metrics; and administering a treatment to the subject participating in space travel at a time point, wherein the treatment and the time point of administering the treatment is determined as an output of a trained predictive model when the trained predictive model is provided the exposomic features of the subject as an input.

In some embodiments, the trained predictive model is configured to output an efficacy of the treatment.

In some embodiments, the trained predictive model is configured to output a measure of whether or not the treatment is efficacious.

In some embodiments, the time point comprises a time prior to, during, after, or any combination thereof time of the subject participating in the space travel.

In some embodiments, the treatment is provided to mitigate negative side-effects of participating in space travel.

In some embodiments, the efficacy of the treatment is determined by the duration of time after the subject's participation in space travel after which the subject's exposomic features comprise baseline exposomic features of a subject that has not participated in space travel.

In some embodiments, the trained predictive model comprises a classification algorithm.

In some embodiments, space travel comprises traveling beyond the Karman line at an altitude of at least 100 kilometers (km) above the Earth's surface.

In some embodiments, the one or more biological samples comprise hair, teeth, toenails, fingernails, physiologic parameters, or any combination thereof.

In some embodiments, the exposomic features comprise a measurement of temporal dynamics of the one or more exposomic signatures.

In some embodiments, the measurement of the temporal dynamics comprises: determination of a linear slope, determination of a plurality of non-linear parameters describing curvature of the one or more exposomic signatures, determination of an abrupt change in intensity of the one or more exposomic signatures, determination of one or more changes in a baseline intensity of the one or more exposomic signatures, determination of a change of a frequency-domain representation of the one or more exposomic signatures, determination of a change of the power-spectral domain representation of the one or more exposomic signatures, determination of one or more recurrence quantification analysis parameters, determination of one or more cross-recurrence quantification analysis parameters, determination of one or more joint recurrence quantification analysis parameters, determination of one or more multidimensional recurrence quantification analysis parameters, estimation of a Lyapunov spectra, determination of a maximum Lyapunov exponent, or any combination thereof.

In some embodiments, the measurement of the temporal dynamics comprises determination of one or more recurrence quantification analysis parameters, wherein the one or more recurrence quantification analysis parameters comprises a recurrence rate, a determinism, a mean diagonal length, a maximum diagonal length, a divergence, a Shannon entropy in diagonal length, a trend in recurrence, a laminarity, a trapping time, a maximum vertical line length, a Shannon entropy in vertical line length, a mean recurrence time, a Shannon entropy in recurrence time, or a number of a most probable recurrence.

In some embodiments, the one or more exposomic signatures are measured by mass spectrometry, laser ablation-inductively coupled plasma-mass spectrometry, laser induced breakdown spectroscopy, Raman spectroscopy, immunohistochemistry fluorescence, or any combination thereof.

In some embodiments, the exposomic features are derived from one or more attractors.

In some embodiments, the method further comprises analyzing a dynamic relationship between the one or more attractors' signal recurrence rates, determinism, mean diagonal length, maximum diagonal length, divergence, Shannon entropy in diagonal length, trend in recurrences, laminarity, trapping time, maximum vertical line length, Shannon entropy in vertical line lengths, mean recurrence time, Shannon entropy in recurrence times, number of the most probable recurrences, or any combination thereof.

In some embodiments, the subject is a human, non-human mammal, or any combination thereof.

In some embodiments, the trained predictive model comprises a classification algorithm.

In some embodiments, the classification algorithm is trained with one or more subjects' one or more biological samples' exposomic features, treatment administered, time point of administering the treatment, and corresponding clinical response, wherein the one or more subjects participated in space travel.

In some embodiments, the classification algorithm is configured to determine if one or more subjects have a dysregulated metabolism induced by the space travel.

In an aspect, the present disclosure provides a method for evaluating one or more subjects' response to an intervention in the presence or lack thereof gravity, comprising: sampling each respective position in a plurality of positions along a reference line of a first set of one or more biological samples of a first set of subjects and a second set of one or more biological samples of a second set of subjects, thereby obtaining a plurality of ion samples, each ion sample in the plurality of ion samples corresponding to a different position in the plurality of positions, and each position in the plurality of positions representing a different period of growth of the first and second set of one or more biological samples, wherein the first set of subjects are subjected to space travel and the second set of subjects are not subjected to space travel, and wherein the first and second set of subjects are administered an intervention; analyzing each ion sample in the plurality of ion samples with a mass spectrometer thereby obtaining a first dataset that includes a plurality of traces, each trace in the plurality of traces being a concentration of a corresponding elemental isotope, in a plurality of elemental isotopes, over time collectively determined from the plurality of ion samples; deriving a second dataset from the plurality of traces that includes a set of features, each respective feature in the set of features being determined by a variation of a single isotope or a combination of isotopes in the plurality of traces; and inputting the set of features into a trained classifier thereby obtaining a probability from the trained classifier of the first and second set of one or more subjects' which predicts the one or more subjects' response to the space travel.

In some embodiments, the probability indicates that a biological sample of the one or more biological samples has been dysregulated as a result of the space travel.

In some embodiments, an efficacy of the intervention is measured by the probability of dysregulation.

In some embodiments, the intervention comprises a diet, pharmaceutical therapeutic, or any combination thereof.

In some embodiments, the diet comprises iodine supplementation.

In some embodiments, the intervention ameliorates radiation exposure.

In some embodiments, the first or second set of subjects comprise a human, non-human mammal, or any combination thereof.

In some embodiments, the non-human mammal comprises mice.

In some embodiments, the first or second set of one or more biological samples comprise hair, teeth, toenails, fingernails, physiologic parameters, or any combination thereof.

In some embodiments, the method further comprises treating the first or second set of one or more biological samples with a solvent or a surfactant prior to the sampling.

In some embodiments, the method further comprises irradiating the first or second set of one or more biological samples with a low powered laser to remove debris from the first or second set of one or more biological samples prior to the sampling.

In some embodiments, the sampling including irradiating, with a laser, the first or second set of one or more biological samples of the first or second set of subjects thereby extracting a plurality of particles from the first or second set of one or more biological samples and ionizing the plurality of particles with an inductively coupled plasma mass spectrometer, thereby obtaining the plurality of ion samples.

In some embodiments, the first or second set of subjects' responses comprise adverse response, positive response, or neutral response.

In some embodiments, the trained classifier is configured to identify a set of features of a subject associated with participating in space travel.

In some embodiments, the trained classifier is trained with the first set of subjects' one or more biological samples' set of features and corresponding responses.

In some embodiments, the trained classifier is configured to determine if a subject comprises a dysregulated metabolism induced by the space travel.

In an aspect, the present disclosure provides a method for outputting a subject's response to an intervention following space travel, comprising: exposing a subject's one or more biological samples to an optical signal comprising a plurality of Raman wavelengths, wherein the subject is subjected to space travel; acquiring a plurality of Raman spectra from the one or more biological samples; processing the plurality of Raman spectra to generate a plurality of Raman features; and outputting a probability of a subject's response to an intervention when subjected to space travel as a result of providing a trained classifier an input of the plurality of Raman features.

In some embodiments, the probability indicates that the subject has been dysregulated as a result of the space travel.

In some embodiments, an efficacy of the intervention is measured by the probability of dysregulation.

In some embodiments, the one or more biological samples comprise hair, teeth, toenails, fingernails, physiologic parameters, or any combination thereof.

In some embodiments, the intervention comprises a diet, pharmaceutical therapeutic, or any combination thereof.

In some embodiments, the diet comprises iodine supplementation.

In some embodiments, the intervention ameliorates radiation exposure.

In some embodiments, the subject comprises a human, non-human mammal, or any combination thereof.

In some embodiments, the non-human mammal comprises mice.

In some embodiments, the plurality of Raman spectra comprises from about 200 to about 3700 wave numbers.

In some embodiments, the acquiring comprises using a Raman spectroscopy microscope.

In some embodiments, the plurality of Raman features comprise one or more traces derived from a Raman spatial map.

In some embodiments, the one or more traces are reduced using a dimensionality-reduction algorithm, wherein the dimensionality-reduction algorithm comprises principal component analyses, independent component analysis, or any combination thereof algorithm.

In some embodiments, a measurement of temporal dynamics is derived from the one or more traces, wherein the temporal dynamics comprise: determination of a linear slope, determination of a plurality of non-linear parameters describing curvature of the one or more exposomic signatures, determination of an abrupt change in intensity of the one or more exposomic signatures, determination of one or more changes in a baseline intensity of the one or more exposomic signatures, determination of a change of a frequency-domain representation of the one or more exposomic signatures, determination of a change of the power-spectral domain representation of the one or more exposomic signatures, determination of one or more recurrence quantification analysis parameters, determination of one or more cross-recurrence quantification analysis parameters, determination of one or more joint recurrence quantification analysis parameters, determination of one or more multidimensional recurrence quantification analysis parameters, estimation of a Lyapunov spectra, determination of a maximum Lyapunov exponent, or any combination thereof.

In some embodiments, the measurement of the temporal dynamics comprises determination of one or more the recurrence quantification analysis parameters, wherein the one or more the recurrence quantification analysis parameters comprises a recurrence rate, a determinism, a mean diagonal length, a maximum diagonal length, a divergence, a Shannon entropy in diagonal length, a trend in recurrence, a laminarity, a trapping time, a maximum vertical line length, a Shannon entropy in vertical line length, a mean recurrence time, a Shannon entropy in recurrence time, or a number of a most probable recurrence.

In some embodiments, the trained classifier comprises a gradient-boosted ensemble model.

In some embodiments, the trained classifier is configured to process one or more features selected from the group consisting of laminarity, entropy, trapping time (TT), mean diagonal length (MDL), recurrence time (RT), Vmax, determinism, Lmax, and any combination thereof.

In some embodiments, the trained classifier is configured to process two or more features selected from the group consisting of laminarity, entropy, trapping time (TT), mean diagonal length (MDL), recurrence time (RT), Vmax, determinism, Lmax, and any combination thereof.

In some embodiments, the trained classifier is trained to identify Raman signatures associated with space travel.

In some embodiments, the output probability of the trained classifier indicates that the subject is exhibiting temporal dynamics associated with space travel.

In some embodiments, the trained classifier is trained with a population of human or non-human animals' plurality of Raman features, wherein the population of human or non-human animals have experienced space travel.

In some embodiments, an efficacy of the intervention is measured by a change of the subject's temporal dynamics towards a baseline temporal dynamics of the subject prior to space travel.

In an aspect, the present disclosure provides a method for evaluating a subject's response to an intervention to reduce the biological effects of space travel, comprising: staining a first set of one or more biological samples of a first set of subject and a second set of one or more biological samples of a second set of subjects to produce a stained first and second set of one or more biological samples, wherein the first set of subject are subjected to space travel and the second set of subjects are not subjected to space travel, and wherein the first and second set of subjects are administered an intervention; analyzing a fluorescence intensity spatially across the stained first and second set of one or more biological samples; and calculating a set of features of the first and second set of subjects' spatial fluorescent intensity across the stained first and second set of one or more biological samples, wherein each feature of the first and second set of subjects' exposomic features comprises one or more metrics quantifying temporal dynamics in the fluorescence intensity trace; and outputting a probability of a subject's response to an intervention to reduce the biological effects of space travel as an output of a trained predictive model when the predictive model is provided the subject's exposomic features of spatial fluorescent intensity across a stained one or more biological samples of the subject, wherein the trained predictive model is trained with the first and second set of subjects' exposomic features' one or more metrics, corresponding administered intervention, and clinical response to the administered intervention.

In some embodiments, the trained predictive model is configured to determine whether a biological sample of the subjects has been dysregulated via space travel.

In some embodiments, the trained predictive model is configured to determine the effect of the intervention as a measure of intervention efficacy based at least in part on the analysis of the fluorescence intensity spatially across one or more biological samples.

In some embodiments, the first or second set of one or more biological samples comprise hair, teeth, toenails, fingernails, physiologic parameters, or any combination thereof.

In some embodiments, the intervention comprises a diet, pharmaceutical therapeutic, or any combination thereof.

In some embodiments, the diet comprises iodine supplementation.

In some embodiments, the intervention ameliorates radiation exposure.

In some embodiments, the first or second set of subjects comprise a human, non-human mammal, or any combination thereof.

In some embodiments, the non-human mammal comprises mice.

In some embodiments, the trained predictive model comprises a gradient-boosted ensemble model.

In some embodiments, a measurement of temporal dynamics of the spatial fluorescence intensity traces include: determination of a linear slope, determination of a plurality of non-linear parameters describing curvature of the one or more exposomic signatures, determination of an abrupt change in intensity of the one or more exposomic signatures, determination of one or more changes in a baseline intensity of the one or more exposomic signatures, determination of a change of a frequency-domain representation of the one or more exposomic signatures, determination of a change of the power-spectral domain representation of the one or more exposomic signatures, determination of one or more recurrence quantification analysis parameters, determination of one or more cross-recurrence quantification analysis parameters, determination of one or more joint recurrence quantification analysis parameters, determination of one or more multidimensional recurrence quantification analysis parameters, estimation of a Lyapunov spectra, determination of a maximum Lyapunov exponent, or any combination thereof.

In some embodiments, the measurement of the temporal dynamics comprises determination of one or more the recurrence quantification analysis parameters, wherein the one or more the recurrence quantification analysis parameters comprises a recurrence rate, a determinism, a mean diagonal length, a maximum diagonal length, a divergence, a Shannon entropy in diagonal length, a trend in recurrence, a laminarity, a trapping time, a maximum vertical line length, a Shannon entropy in vertical line length, a mean recurrence time, a Shannon entropy in recurrence time, or a number of a most probable recurrence.

In some embodiments, the analyzing comprises obtaining a fluorescence image of the first and second set stained one or more biological samples and analyzing the spatial fluorescence intensity of the fluorescence image.

In some embodiments, obtaining the fluorescence image of the stained tooth sample comprises using an inverted or non-inverted confocal microscope.

In some embodiments, staining the first set of one or more biological samples of a first and second set of subjects comprises using a C-reactive stain.

In some embodiments, the method further comprises sectioning the tooth sample.

In some embodiments, staining the first and second set of one or more biological samples comprises decalcifying the first and second set of one or more biological samples.

In some embodiments, the trained model is selected from the group consisting of: a neural network algorithm, a support vector machine algorithm, a decision tree algorithm, an unsupervised clustering algorithm, a supervised clustering algorithm, a regression algorithm, a gradient-boosting algorithm, and any combination thereof.

In some embodiments, the trained model comprises a gradient-boosted decision tree.

In some embodiments, the trained predictive model comprises a classification algorithm.

In some embodiments, the classification algorithm is trained to identify features in spatial fluorescence intensity associated with the space travel.

In an aspect, the present disclosure provides a method for outputting one or more pathways associated with space travel, comprising: receiving one or more biological samples of a first set of one or more subjects and a second set of one or more subjects, wherein the first set of one or more subjects has participated in space travel and the second set of one or more subjects has not; determining one or more pathways' exposomic signatures from the one or more biological samples of the first and second set of one or more subjects; calculating exposomic features of the one or more pathways' exposomic signatures; and outputting the one or more pathways associated with space travel by comparing the first and second set of subjects' exposomic features of the one or more pathways.

In some embodiments, each feature of the exposomic features comprises one or more quantitative metrics.

In some embodiments, space travel comprises traveling beyond the Karman line at an altitude of at least 100 kilometers (km) above the Earth's surface.

In some embodiments, the biological sample comprises hair, teeth, toenails, fingernails, physiologic parameters, or any combination thereof.

In some embodiments, the exposomic features comprise a measurement of temporal dynamics of the one or more exposomic signatures.

In some embodiments, the measurement of the temporal dynamics comprises: determination of a linear slope, determination of a plurality of non-linear parameters describing curvature of the one or more exposomic signatures, determination of an abrupt change in intensity of the one or more exposomic signatures, determination of one or more changes in a baseline intensity of the one or more exposomic signatures, determination of a change of a frequency-domain representation of the one or more exposomic signatures, determination of a change of the power-spectral domain representation of the one or more exposomic signatures, determination of one or more recurrence quantification analysis parameters, determination of one or more cross-recurrence quantification analysis parameters, determination of one or more joint recurrence quantification analysis parameters, determination of one or more multidimensional recurrence quantification analysis parameters, estimation of a Lyapunov spectra, determination of a maximum Lyapunov exponent, or any combination thereof.

In some embodiments, the measurement of the temporal dynamics comprises determination of one or more recurrence quantification analysis parameters, wherein the one or more recurrence quantification analysis parameters comprises a recurrence rate, a determinism, a mean diagonal length, a maximum diagonal length, a divergence, a Shannon entropy in diagonal length, a trend in recurrence, a laminarity, a trapping time, a maximum vertical line length, a Shannon entropy in vertical line length, a mean recurrence time, a Shannon entropy in recurrence time, or a number of a most probable recurrence.

In some embodiments, the exposomic signatures are measured by mass spectrometry, laser ablation-inductively coupled plasma-mass spectrometry, laser induced breakdown spectroscopy, Raman spectroscopy, immunohistochemistry fluorescence, or any combination thereof.

In some embodiments, the exposomic features are derived from one or more attractors.

In some embodiments, the method further comprises analyzing a dynamic relationship between the one or more signal recurrence rates, determinism, mean diagonal length, maximum diagonal length, divergence, Shannon entropy in diagonal length, trend in recurrences, laminarity, trapping time, maximum vertical line length, Shannon entropy in vertical line lengths, mean recurrence time, Shannon entropy in recurrence times, number of the most probable recurrences, or any combination thereof.

In some embodiments, the one or more subjects are a human, non-human mammal, vertebrate, or any combination thereof.

In an aspect, the present disclosure provides a method for providing a treatment to ameliorate the effects of space travel, comprising: receiving one or more biological samples of one or more subjects participating in space travel; determining one or more pathways' exposomic signatures from the one or more biological samples of the one or more subjects; calculating exposomic features of the one or more pathways' exposomic signatures; and providing a treatment to the one or more subjects, wherein the treatment ameliorates the effect of the space travel on the one or more subjects' exposomic features of the one or more pathways' exposomic signatures.

In some embodiments, the treatment comprises a diet, pharmaceutical, nutraceutical, food bar, or any combination thereof.

In some embodiments, each feature of the exposomic features comprises one or more quantitative metrics.

In some embodiments, the space travel comprises traveling beyond the Karman line at an altitude of at least 100 kilometers (km) above the Earth's surface.

In some embodiments, the biological sample comprises hair, teeth, toenails, fingernails, physiologic parameters, or any combination thereof.

In some embodiments, the exposomic features comprise a measurement of temporal dynamics of the one or more exposomic signatures.

In some embodiments, the measurement of the temporal dynamics comprises: determination of a linear slope, determination of a plurality of non-linear parameters describing curvature of the one or more exposomic signatures, determination of an abrupt change in intensity of the one or more exposomic signatures, determination of one or more changes in a baseline intensity of the one or more exposomic signatures, determination of a change of a frequency-domain representation of the one or more exposomic signatures, determination of a change of the power-spectral domain representation of the one or more exposomic signatures, determination of one or more recurrence quantification analysis parameters, determination of one or more cross-recurrence quantification analysis parameters, determination of one or more joint recurrence quantification analysis parameters, determination of one or more multidimensional recurrence quantification analysis parameters, estimation of a Lyapunov spectra, determination of a maximum Lyapunov exponent, or any combination thereof.

In some embodiments, the measurement of the temporal dynamics comprises determination of one or more recurrence quantification analysis parameters, wherein the one or more recurrence quantification analysis parameters comprises a recurrence rate, a determinism, a mean diagonal length, a maximum diagonal length, a divergence, a Shannon entropy in diagonal length, a trend in recurrence, a laminarity, a trapping time, a maximum vertical line length, a Shannon entropy in vertical line length, a mean recurrence time, a Shannon entropy in recurrence time, or a number of a most probable recurrence.

In some embodiments, the exposomic signatures are measured by mass spectrometry, laser ablation-inductively coupled plasma-mass spectrometry, laser induced breakdown spectroscopy, Raman spectroscopy, immunohistochemistry fluorescence, or any combination thereof.

In some embodiments, the exposomic features are derived from one or more attractors.

In some embodiments, the method further comprises analyzing a dynamic relationship between the one or more signal recurrence rates, determinism, mean diagonal length, maximum diagonal length, divergence, Shannon entropy in diagonal length, trend in recurrences, laminarity, trapping time, maximum vertical line length, Shannon entropy in vertical line lengths, mean recurrence time, Shannon entropy in recurrence times, number of the most probable recurrences, or any combination thereof.

In some embodiments, the one or more subjects are a human, non-human mammal, vertebrate, or any combination thereof.

Aspects of the disclosure describe a method for outputting one or more pathways associated with an intervention, comprising: (a) receiving one or more biological samples of a first set of one or more subjects and a second set of one or more subjects, wherein the first set of one or more subjects has received an intervention and the second set of one or more subjects has not received an intervention; (b) determining one or more pathways' exposomic signatures from the one or more biological samples of the first and second set of one or more subjects; (c) calculating exposomic features of the one or more pathways' exposomic signatures; and (d) outputting the one or more pathways associated with the intervention by comparing the first and second set of subjects' exposomic features of the one or more pathways. In some embodiments, each feature of the exposomic features comprises one or more quantitative metrics. In some embodiments, the biological sample comprises hair, teeth, toenails, fingernails, physiologic parameters, or any combination thereof. In some embodiments, the one or more subjects are a human, non-human mammal, vertebrate, or any combination thereof. In some embodiments, the intervention comprises a pharmaceutical, diet, food bar, nutraceutical, metal ion supplement, or any combination thereof.

In some embodiments, the exposomic features comprise a measurement of temporal dynamics of the one or more exposomic signatures. In some embodiments, the measurement of the temporal dynamics comprises: determination of a linear slope, determination of a plurality of non-linear parameters describing curvature of the one or more exposomic signatures, determination of an abrupt change in intensity of the one or more exposomic signatures, determination of one or more changes in a baseline intensity of the one or more exposomic signatures, determination of a change of a frequency-domain representation of the one or more exposomic signatures, determination of a change of the power-spectral domain representation of the one or more exposomic signatures, determination of one or more recurrence quantification analysis parameters, determination of one or more cross-recurrence quantification analysis parameters, determination of one or more joint recurrence quantification analysis parameters, determination of one or more multidimensional recurrence quantification analysis parameters, estimation of a Lyapunov spectra, determination of a maximum Lyapunov exponent, or any combination thereof. In some embodiments, the measurement of the temporal dynamics comprises determination of one or more recurrence quantification analysis parameters, wherein the one or more recurrence quantification analysis parameters comprises a recurrence rate, a determinism, a mean diagonal length, a maximum diagonal length, a divergence, a Shannon entropy in diagonal length, a trend in recurrence, a laminarity, a trapping time, a maximum vertical line length, a Shannon entropy in vertical line length, a mean recurrence time, a Shannon entropy in recurrence time, or a number of a most probable recurrence.

In some embodiments, the exposomic signatures are measured by mass spectrometry, laser ablation-inductively coupled plasma-mass spectrometry, laser induced breakdown spectroscopy, Raman spectroscopy, immunohistochemistry fluorescence, or any combination thereof.

In some embodiments, the exposomic features are derived from one or more attractors. In some embodiments, the method further comprises analyzing a dynamic relationship between the one or more signal recurrence rates, determinism, mean diagonal length, maximum diagonal length, divergence, Shannon entropy in diagonal length, trend in recurrences, laminarity, trapping time, maximum vertical line length, Shannon entropy in vertical line lengths, mean recurrence time, Shannon entropy in recurrence times, number of the most probable recurrences, or any combination thereof.

Aspects of the disclosure provide a method a method to determine exposomic features of a subject associated with space travel, comprising: (a) receiving one or more biological samples of a subject; (b) determining one or more pathways' exposomic signatures from the one or more biological samples; (c) calculating exposomic features of the one or more pathways' exposomic signatures; and (d) outputting the exposomic features of the subject associated with space travel by comparing the subject's exposomic features and a database of space travel exposomic features. In some embodiments, each feature of the exposomic features comprises one or more quantitative metrics. In some embodiments, the biological sample comprises hair, teeth, toenails, fingernails, physiologic parameters, or any combination thereof. In some embodiments, the subject is human, non-human mammal, vertebrate, or any combination thereof.

In some embodiments, the exposomic features comprise a measurement of temporal dynamics of the one or more exposomic signatures. In some embodiments, the measurement of the temporal dynamics comprises: determination of a linear slope, determination of a plurality of non-linear parameters describing curvature of the one or more exposomic signatures, determination of an abrupt change in intensity of the one or more exposomic signatures, determination of one or more changes in a baseline intensity of the one or more exposomic signatures, determination of a change of a frequency-domain representation of the one or more exposomic signatures, determination of a change of the power-spectral domain representation of the one or more exposomic signatures, determination of one or more recurrence quantification analysis parameters, determination of one or more cross-recurrence quantification analysis parameters, determination of one or more joint recurrence quantification analysis parameters, determination of one or more multidimensional recurrence quantification analysis parameters, estimation of a Lyapunov spectra, determination of a maximum Lyapunov exponent, or any combination thereof. In some embodiments, the measurement of the temporal dynamics comprises determination of one or more recurrence quantification analysis parameters, wherein the one or more recurrence quantification analysis parameters comprises a recurrence rate, a determinism, a mean diagonal length, a maximum diagonal length, a divergence, a Shannon entropy in diagonal length, a trend in recurrence, a laminarity, a trapping time, a maximum vertical line length, a Shannon entropy in vertical line length, a mean recurrence time, a Shannon entropy in recurrence time, or a number of a most probable recurrence. In some embodiments, the exposomic features are derived from one or more attractors. In some embodiments, the method further comprises analyzing a dynamic relationship between the one or more signal recurrence rates, determinism, mean diagonal length, maximum diagonal length, divergence, Shannon entropy in diagonal length, trend in recurrences, laminarity, trapping time, maximum vertical line length, Shannon entropy in vertical line lengths, mean recurrence time, Shannon entropy in recurrence times, number of the most probable recurrences, or any combination thereof.

In some embodiments, the exposomic signatures are measured by mass spectrometry, laser ablation-inductively coupled plasma-mass spectrometry, laser induced breakdown spectroscopy, Raman spectroscopy, immunohistochemistry fluorescence, or any combination thereof.

Aspects of the disclosure describe a method to determine exposomic features of a subject associated with space travel with a trained predictive model, comprising: (a) receiving one or more biological samples of a subject; (b) determining one or more pathways' exposomic signatures from the one or more biological samples; (c) calculating exposomic features of the one or more pathways' exposomic signatures; and (d) outputting the exposomic features of the subject associated with space travel by providing a trained predictive model with the exposomic features of the subject as an input, wherein the trained predictive model is trained on exposomic features of one or more subjects that participated in space travel. In some embodiments, the biological sample comprises hair, teeth, toenails, fingernails, physiologic parameters, or any combination thereof. In some embodiments, the subject is human, non-human mammal, vertebrate, or any combination thereof.

In some embodiments, each feature of the exposomic features comprises one or more quantitative metrics. In some embodiments, the exposomic features are derived from one or more attractors. In some embodiments, the exposomic features comprise a measurement of temporal dynamics of the one or more exposomic signatures. In some embodiments, the measurement of the temporal dynamics comprises: determination of a linear slope, determination of a plurality of non-linear parameters describing curvature of the one or more exposomic signatures, determination of an abrupt change in intensity of the one or more exposomic signatures, determination of one or more changes in a baseline intensity of the one or more exposomic signatures, determination of a change of a frequency-domain representation of the one or more exposomic signatures, determination of a change of the power-spectral domain representation of the one or more exposomic signatures, determination of one or more recurrence quantification analysis parameters, determination of one or more cross-recurrence quantification analysis parameters, determination of one or more joint recurrence quantification analysis parameters, determination of one or more multidimensional recurrence quantification analysis parameters, estimation of a Lyapunov spectra, determination of a maximum Lyapunov exponent, or any combination thereof. In some embodiments, the measurement of the temporal dynamics comprises determination of one or more recurrence quantification analysis parameters, wherein the one or more recurrence quantification analysis parameters comprises a recurrence rate, a determinism, a mean diagonal length, a maximum diagonal length, a divergence, a Shannon entropy in diagonal length, a trend in recurrence, a laminarity, a trapping time, a maximum vertical line length, a Shannon entropy in vertical line length, a mean recurrence time, a Shannon entropy in recurrence time, or a number of a most probable recurrence. In some embodiments, the method further comprises analyzing a dynamic relationship between the one or more signal recurrence rates, determinism, mean diagonal length, maximum diagonal length, divergence, Shannon entropy in diagonal length, trend in recurrences, laminarity, trapping time, maximum vertical line length, Shannon entropy in vertical line lengths, mean recurrence time, Shannon entropy in recurrence times, number of the most probable recurrences, or any combination thereof.

In some embodiments, the exposomic signatures are measured by mass spectrometry, laser ablation-inductively coupled plasma-mass spectrometry, laser induced breakdown spectroscopy, Raman spectroscopy, immunohistochemistry fluorescence, or any combination thereof.

Another aspect of the present disclosure provides a system comprising one or more computer processors and computer memory coupled thereto. The computer memory comprises machine executable code that, upon execution by the one or more computer processors, implements any of the methods above or elsewhere herein.

Additional aspects and advantages of the present disclosure will become readily apparent to those skilled in the art from the following detailed description, where only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings (also “Figure” and “FIG.” and “FIGS.” herein), of which:

FIG. 1 shows an overview of the computer implemented exposomic system, as seen in some embodiments.

FIGS. 2A-B show the data workflow for training an intervention predictive model using subjects' clinical database data (clinical meta data), exposome biochemical signature, and intervention outcomes (FIG. 2A) and using the trained predictive model (FIG. 2B) to, for example, predict a given subject's intervention outcome, as seen in some embodiments.

FIGS. 3A-B show the data workflow for training a pharmaceutical and or nutraceutical optimal selection predictive model using subjects' clinical database data (clinical meta data), exposomic features, percent difference between subject's post-treatment exposomic features and reference exposomic features. FIG. 3A shows the training workflow to train the predictive model, and FIG. 3B shows the use of the trained predictive model, as seen in some embodiments.

FIG. 4 illustrates a flow diagram for selecting subjects for an intervention based on their exposome biochemical signature profile, as seen in some embodiments.

FIG. 5 shows a flow diagram for selecting optimal pharmaceutical or nutraceutical treatments based on a comparison of exposomic features of a subjects in comparison to a reference treatment exposomic features, as seen in some embodiments herein.

FIGS. 6A-D show exposome biochemical profiles for various exposome signatures (e.g., tin, lead, calcium, and magnesium) for a subject that has not received an intervention (blue) and for a subject that has received an intervention (orange, grey, and teal), as seen in some embodiments herein.

FIGS. 7A-7B illustrate clustering of one or more subjects' one or more exposome biochemical profiles (FIG. 7A) and how such clustering of data may be correlated one or more subjects having participated in space travel (FIG. 7B), as seen in some embodiments herein.

FIG. 8 shows sub-typing of subjects using exposomic features derived from hair analysis. Exposome biochemical signature data were extracted via analytical methods disclosed elsewhere herein. Clustering analysis is shown to identify discrete subtypes of patients that have participated in space travel, as seen in some embodiments herein.

FIG. 9 illustrates the systems and methods utilized to collect and analyze geographical temporal dynamics of annotated exposome pathways through a deep data science framework, as seen in some embodiments herein.

FIG. 10 shows the temporal aspect of obtaining 100 data timepoints from a single biological sample characterizing the dynamics of physiology at different life stages, as seen in some embodiments herein.

FIG. 11 shows the various chemical signatures and their respective grouping measured by the methods and systems, as seen in some embodiments herein.

FIG. 12 shows both temporal and spatial immunohistochemistry (IHC) fluorescence data captured by methods and systems described herein.

FIG. 13 shows a method of measuring metal chemical biomarkers of teeth and correlating the spatial distribution of the metal chemical biomarkers across teeth growth lines to determine the effects of space travel and changes in biochemical physiology, etc., as seen in some embodiments herein.

FIG. 14 shows machine learning, informatics, and deep learning platform configured to generate robust and generalizable predictive models of space travel.

FIG. 15 shows a method of phenotyping, pathway identification, metabolic phenotyping, and clinical sub-typing of various physiological outcomes by unsupervised pattern recognition of an exposome map.

FIG. 16 shows impacted probiotic metabolic and corresponding biochemical pathways measured by the methods and systems, as seen in some embodiments herein.

FIG. 17 shows impacted gluten metabolic and corresponding biochemical pathways measured by the methods and systems, as seen in some embodiments herein.

FIG. 18 shows the pathway importance weight from subjects who participated in space travel utilized by the methods and systems described to recommend pharmaceutical and nutraceutical compounds to ameliorate the negative effects of space travel.

FIGS. 19A-C show various forms of exposomic signature data representation, as seen in some embodiments herein.

FIGS. 20A-D show the comparison of calcium (FIGS. 20A-B) and copper (FIGS. 20C-D) exposomic signatures and their corresponding attractor graphical representations, as seen in some embodiments.

FIGS. 21A-B illustrate recurrence networks, as seen in some embodiments.

FIG. 22 illustrates a flow diagram for a method of outputting one or more quantitative metrics of a subject's one or more exposomic signatures, as seen in some embodiments.

FIG. 23 illustrate a flow diagram of a method for outputting a prediction of one or more subjects' phenotypic data, as seen in some embodiments.

FIG. 24 shows quantitative metrics derived from cross-recurrent quantification analysis of signal dyads from control animals housed in a ground facility and mice that traveled to the international space Station, as seen in some embodiments.

FIG. 25 shows data derived from analysis of barium, manganese, and zinc signals of human hair collected after space travel, as seen in some embodiments.

FIG. 26 shows mean Ba: CA dynamics, Zn: Fe dynamics in mice that received a control diet or received iodine supplementation to their diet, as seen in some embodiments.

FIG. 27 shows mean Ba: CA dynamics, Zn: Fe dynamics in mice that received a diet of food bars or received a standard lab chow diet, as seen in some embodiments.

### DETAILED DESCRIPTION

While various embodiments have been shown and described herein, it will be obvious to those skilled in the art that such embodiments are provided by way of example only. Numerous variations, changes, and substitutions may occur to those skilled in the art without departing from the disclosure. It should be understood that various alternatives to the embodiments of the disclosure described herein may be employed.

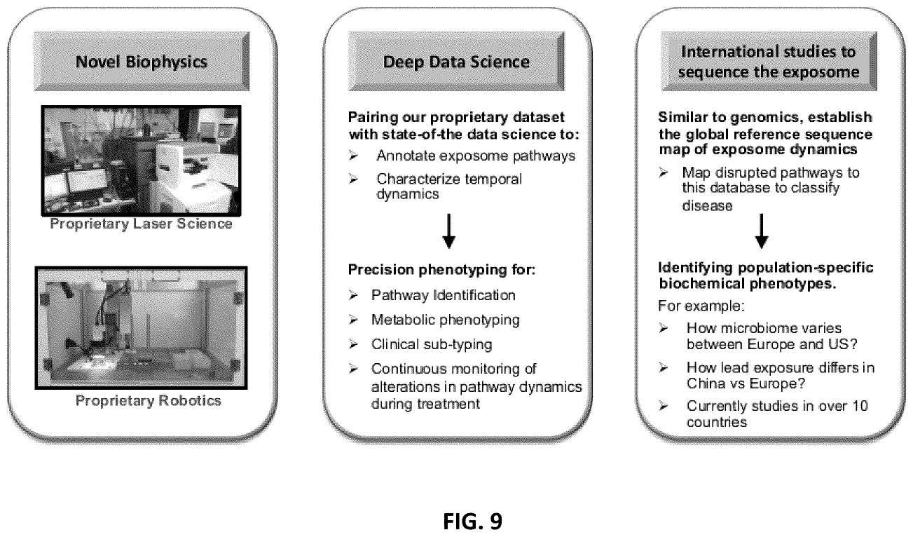

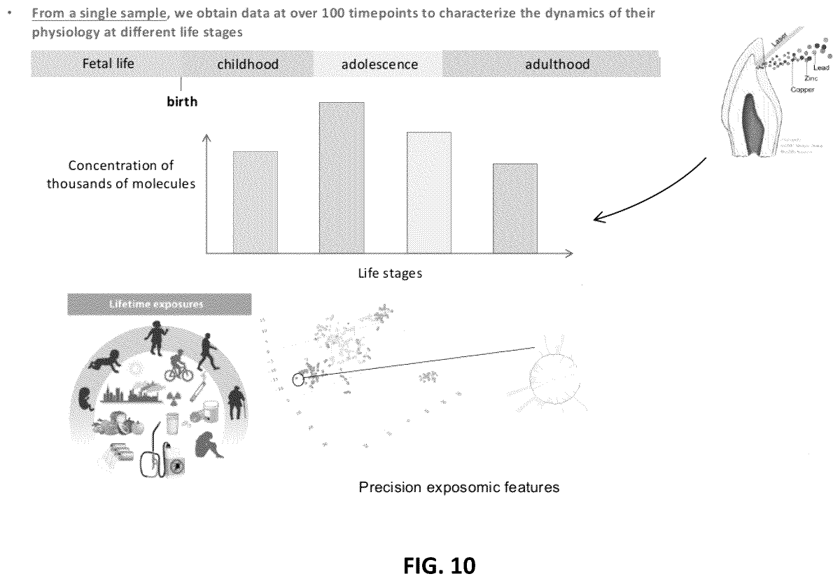

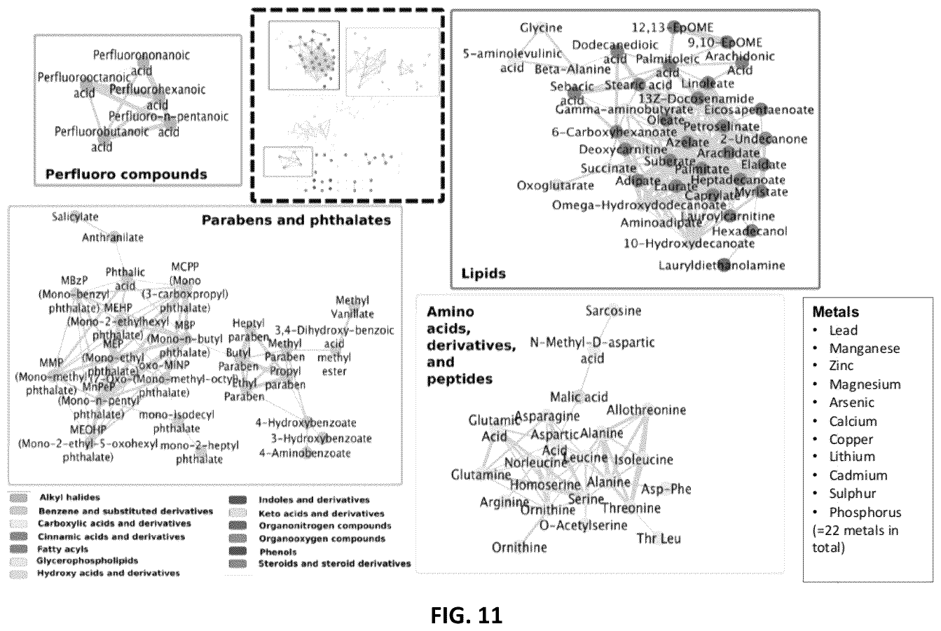

The present disclosure addresses these needs through systems and methods capable of analyzing and classifying subjects by their exposome biochemical signature profile, as shown in FIG. 9. The exposomic feature analysis of the present disclosure may comprise analyzing over 50,000 biochemical signatures (FIG. 11) in a non-invasive manner from a hair shaft, tooth, fingernail, toenail, physiologic parameter, or any combination thereof. With such systems and methods of analysis of the present disclosure, subtle changes in a subject's biochemistry induced by, for example, diet, air-pollution, psychological stress, space travel, exposure to pesticides, or industrial chemical (FIG. 10) to name a few factors, may be investigated and correlated to intervention response outcomes and targeted highly efficacious pharmaceuticals and nutraceuticals.

In addition to the copious dataset generated by exposomic biochemical signature analysis, exposomic biochemical signature analysis can also provide insight into temporal fluctuations of said biochemical signatures over the span of a subject's life, as shown in FIG. 10. Such an approach may be utilized to screen individuals participating in space travel to determine what single or combination of exposomic features are affected by space travel. The identified pathways may then be used to train statistical, machine learning, and/or artificial intelligence predictive models capable of predicting whether a subjects exposomic features are characteristic of an individual that has participated in space travel.

### Computer Implemented Exposomic System

In an aspect, the present disclosure provides a computer implemented exposomic system for gathering, storing, cataloguing, comparing, analyzing, or any combination thereof, exposomic biochemical signatures for one or more subjects. In some embodiments, the exposomic biochemical signatures may be used at least in part for optimizing selection criterion for subjects participating in interventions. In some embodiments, the exposomic biochemical signatures is used at least in part for suggesting optimal pharmaceutical or nutraceutical treatment for subjects in need thereof. In some embodiments, intervention may comprise a clinical trial, community trial, or any combination thereof.

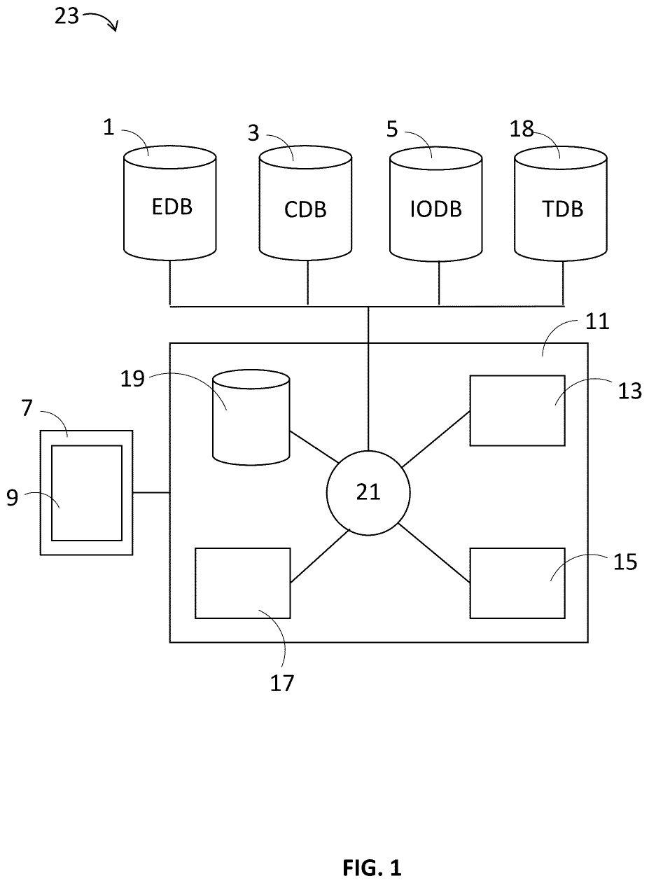

Turning to FIG. 1, the computer implemented exposomic system 23 may comprise one or more of the following: (a) an exposome biochemical signatures database (EDB) 1, the EDB may further comprise biochemical signature information for a plurality of subjects that have and have not participated in space travel; (b) a clinical database (CBD) 3, the CBD may further comprise clinical phenotype information for the plurality of subjects that have and have not participated in space travel; (c) an intervention requirement database (IODB) 5, the IODB may further comprise information on intervention outcome information for at least one phase of at least one intervention; (d) a treatment database (TDB) 18; and (e) a computer system 11 that may comprise a processing unit (CPU, also “processor” and “computer processor” herein) 21, which can be a single core or a multi core processor, or a plurality of processor for parallel processing. The processor 21 may execute a sequence of machine-readable instructions embodied in a program or software, for example, (i) an association software module located on storage unit 19 i.e., memory, communicatively coupled to the EDB 1 and the CDB 3, the association software module may be programmed to determine an association between the exposomic features and the clinical phenotype information for at least one of the plurality of subjects, and (ii) a recommendation software module located on memory 19. The software may be loaded from the memory 19 into random access memory (RAM) 17 or read-only memory (ROM) 17 that may provide an intervention recommendation for the at least one of the plurality of subjects based at least in part on the exposomic features, the clinical phenotype information, the intervention outcome information, and the association between the exposomic features and the clinical phenotype information for the at least one of the plurality of subjects.

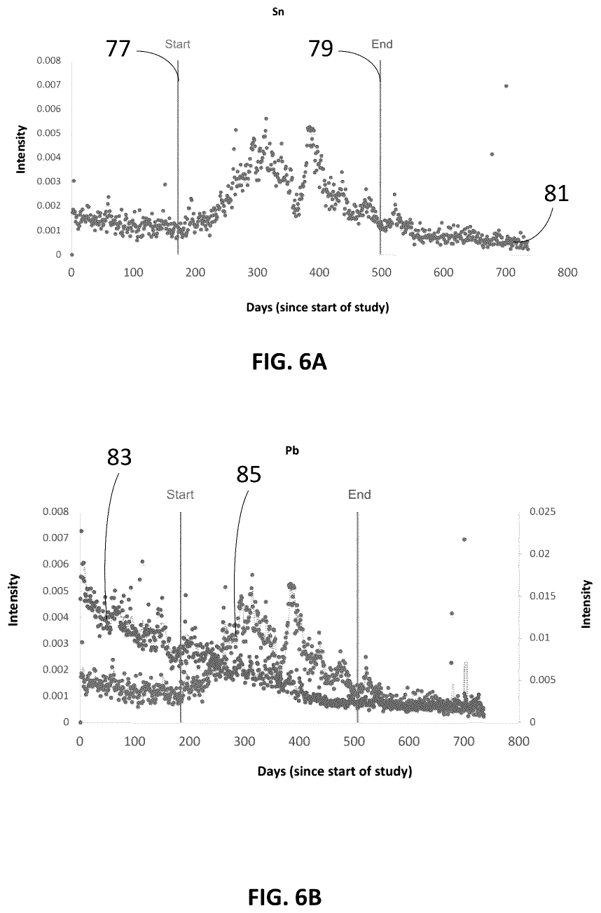

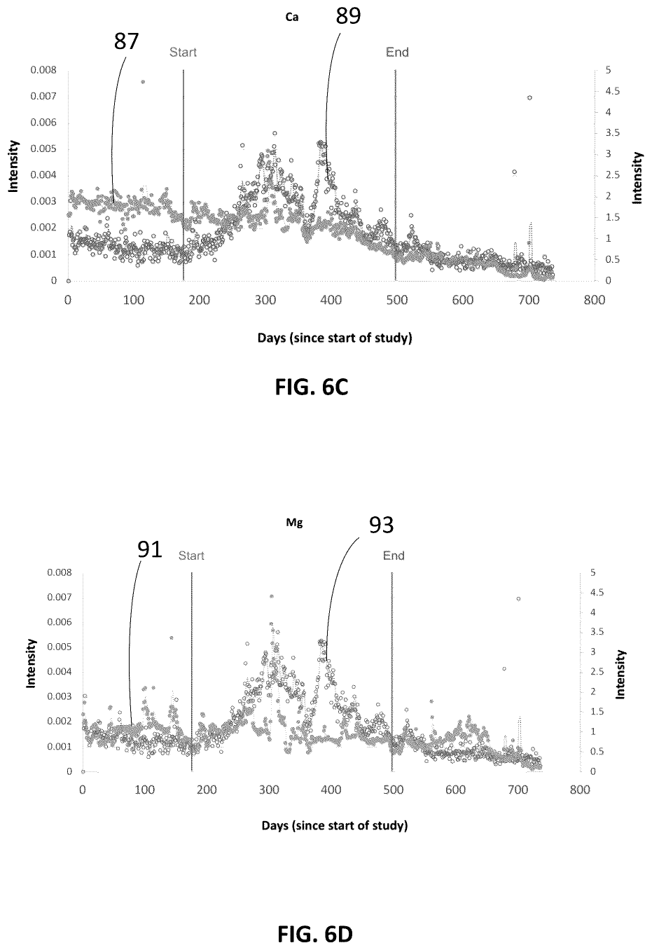

In some embodiments, the exposome biochemical signatures database (EDB) 1 may comprise exposomic features from a plurality of subjects that have and have not participated in space travel. Exposome biochemical signatures may comprise biochemical signatures of perfluoro compounds, parabens, phthalates, lipids, amino acids, metabolites, peptides, metals, derivatives thereof, or any combination thereof, as seen in FIG. 11. In some embodiments, exposome biochemical signatures are analyzed or acquired as a function of a subjects' lives (e.g., as a function of aging or as a function of time), in this case, the collection of exposome biochemical signatures, as seen in FIG. 6A-6D, may be analyzed to produce one or more exposomic features. In some embodiments, the period of time represented by an exposome biochemical signature may comprise at least 1 hour, at least 1 day, at least 1 week, at least 1 month, at least 1 year, or any combination thereof.

In some embodiments, the number of exposome biochemical signatures may comprise about 10 signatures to about 100,000 signatures. In some embodiments, the number of exposome biochemical signatures may comprise about 10 signatures to about 100 signatures, about 10 signatures to about 500 signatures, about 10 signatures to about 1,000 signatures, about 10 signatures to about 5,000 signatures, about 10 signatures to about 7,000 signatures, about 10 signatures to about 10,000 signatures, about 10 signatures to about 20,000 signatures, about 10 signatures to about 50,000 signatures, about 10 signatures to about 100,000 signatures, about 100 signatures to about 500 signatures, about 100 signatures to about 1,000 signatures, about 100 signatures to about 5,000 signatures, about 100 signatures to about 7,000 signatures, about 100 signatures to about 10,000 signatures, about 100 signatures to about 20,000 signatures, about 100 signatures to about 50,000 signatures, about 100 signatures to about 100,000 signatures, about 500 signatures to about 1,000 signatures, about 500 signatures to about 5,000 signatures, about 500 signatures to about 7,000 signatures, about 500 signatures to about 10,000 signatures, about 500 signatures to about 20,000 signatures, about 500 signatures to about 50,000 signatures, about 500 signatures to about 100,000 signatures, about 1,000 signatures to about 5,000 signatures, about 1,000 signatures to about 7,000 signatures, about 1,000 signatures to about 10,000 signatures, about 1,000 signatures to about 20,000 signatures, about 1,000 signatures to about 50,000 signatures, about 1,000 signatures to about 100,000 signatures, about 5,000 signatures to about 7,000 signatures, about 5,000 signatures to about 10,000 signatures, about 5,000 signatures to about 20,000 signatures, about 5,000 signatures to about 50,000 signatures, about 5,000 signatures to about 100,000 signatures, about 7,000 signatures to about 10,000 signatures, about 7,000 signatures to about 20,000 signatures, about 7,000 signatures to about 50,000 signatures, about 7,000 signatures to about 100,000 signatures, about 10,000 signatures to about 20,000 signatures, about 10,000 signatures to about 50,000 signatures, about 10,000 signatures to about 100,000 signatures, about 20,000 signatures to about 50,000 signatures, about 20,000 signatures to about 100,000 signatures, or about 50,000 signatures to about 100,000 signatures. In some embodiments, the number of exposome biochemical signatures may comprise about 10 signatures, about 100 signatures, about 500 signatures, about 1,000 signatures, about 5,000 signatures, about 7,000 signatures, about 10,000 signatures, about 20,000 signatures, about 50,000 signatures, or about 100,000 signatures. In some embodiments, the number of exposome biochemical signatures may comprise at least about 10 signatures, about 100 signatures, about 500 signatures, about 1,000 signatures, about 5,000 signatures, about 7,000 signatures, about 10,000 signatures, about 20,000 signatures, or about 50,000 signatures. In some embodiments, the number of exposome biochemical signatures may comprise at most about 100 signatures, about 500 signatures, about 1,000 signatures, about 5,000 signatures, about 7,000 signatures, about 10,000 signatures, about 20,000 signatures, about 50,000 signatures, or about 100,000 signatures.

In some embodiments, the exposome biochemical signatures may be obtained by assaying biological samples of a plurality of subjects. In some embodiments, the biological samples may comprise a tooth, nail, or hair shaft sample. In some embodiments, the exposome biochemical signatures may be obtained using laser ablation-inductively coupled plasma mass spectrometry measurements, laser induced breakdown spectroscopy measurements, mass spectrometry measurements, Raman spectroscopy measurements, immunohistochemistry measurements, molecular tagging (with a fluorophore, for example), nuclear magnetic resonance, chromatography, or any combination thereof. In some embodiments, laser ablation-inductively coupled plasma mass spectrometry measurements may measure one or more element chemicals. In some embodiments, the one or more element chemicals comprise aluminum, arsenic, barium, bismuth, calcium, copper, iodide, lead, lithium, magnesium, manganese, phosphorus, sulfur, tin, strontium, zinc, or any combination thereof chemicals as described elsewhere herein e.g., Table 1 and/or Table 2. In some embodiments, the exposome biochemical signatures information may comprise exposome temporal biochemical responses of the plurality of subjects. In some embodiments, the biochemical information may comprise fluorescence images of the biological samples. In some embodiments, the exposome biochemical signatures may comprise spatial maps of Raman spectra of the biological samples of the plurality of subjects. In some embodiments, the exposomic features may be associated with space travel.

In some embodiments, the plurality of chemicals is selected from the chemicals listed in Table 1. In some embodiments, the plurality of chemicals includes at least 50%, 60%, 70%, 80% or 90% of the isotopes included in Table 1.

TABLE 1
List of Chemicals

| Chemicals | Element Name |
|---|---|
| Li-7 (Li) | lithium |
| Mg-24 (Mg) | magnesium |
| Mg-25 (Mg25) | magnesium |
| Al-27 (Al) | aluminum |
| P-31 (P) | phosphorus |
| S-34 (S) | sulfur |
| Ca-44 (Ca) | calcium |
| Ca-43 (Ca43) | calcium |
| Cr-52 (Cr) | chromium |
| Mn-55 (Mn) | manganese |
| Fe-56 (Fe) | iron |
| Co-59 (Co) | cobalt |
| Ni-60 (Ni) | nickel |
| Cu-63 (Cu) | copper |
| Zn-66 (Zn) | zinc |
| As-75 (As) | arsenic |
| Sr-88 (Sr) | strontium |
| Cd-111 (Cd) | cadmium |
| Sn-118 (Sn) | tin |
| I-127 (I) | iodine |
| Ba-138 (Ba) | barium |
| Hg-201 (Hg) | mercury |
| Pb-208 (Pb) | lead |
| Bi-209 (Bi) | bismuth |
| Mo-95(Mo) | molybdenum |

In some embodiments, the plurality of chemicals is selected from the chemicals listed in Table 2. In some embodiments, the plurality of chemicals includes at least 50%, 60%, 70%, 80% or 90% of the isotopes included in Table 2.

TABLE 2
List of Chemicals

| Chemicals | Element Name |
|---|---|
| Li7 | lithium |
| Mg24 | magnesium |
| Al27 | aluminum |
| P31 | phosphorus |
| S34 | sulfur |
| Ca44 | calcium |
| V51 | vanadium |
| Cr52 | chromium |
| Mn55 | manganese |
| Fe56, Fe57 | iron |
| Co59 | cobalt |
| Ni60 | nickel |
| Cu63 | copper |
| Zn66 | zinc |
| As75 | arsenic |
| Sr88 | strontium |
| Cd111 | cadmium |
| Sn118 | tin |
| Sb121 | antimony |
| I127 | iodine |
| Ba138 | barium |
| Hg201 | mercury |
| Pb208 | lead |
| Bi209 | bismuth |

In some embodiments, one or more exposomic features are calculated from one or more dynamic exposomic biochemical signatures. Exposomic features derived through data analysis may comprise descriptive statistics or parameters which are utilized in subsequent statistical, machine learning, or artificial intelligence models. Such exposomic features may comprise standard descriptive metrics such as the mean, median, mode, and range, and/or associated measures of error and/or variation such as standard deviation, variance, confidence intervals, and/or related metrics of the one or more dynamic exposomic signatures. The derivation of exposomic features may comprise the application of computational methods to derive linear slope, non-linear parameters describing curvature of the one or more dynamic exposomic signatures, abrupt changes in intensity of the one or more dynamic exposomic signatures, changes in baseline intensity of the one or more dynamic exposomic signatures, changes of the frequency-domain representation of the one or more dynamic exposomic signatures, changes of the power-spectral domain representation of the one or more dynamic exposomic signatures, recurrence quantification analysis parameters, cross-recurrence quantification analysis parameters, joint recurrence quantification analysis parameters, multidimensional recurrence quantification analysis parameters, estimation of the Lyapunov spectra or maximum Lyapunov exponent or any combination thereof.

In some embodiments, the one or more exposomic features comprise a measurement of temporal dynamics of the one or more dynamic exposomic signatures. In some embodiments, the measurement of the temporal dynamics comprise: linear slope, non-linear parameters describing curvature of the one or more dynamic exposomic signatures, abrupt changes in intensity of the one or more dynamic exposomic signatures, changes in baseline intensity of the one or more dynamic exposomic signatures, changes of the frequency-domain representation of the one or more dynamic exposomic signatures, changes of the power-spectral domain representation of the one or more dynamic exposomic signatures, recurrence quantification analysis parameters, cross-recurrence quantification analysis parameters, joint recurrence quantification analysis parameters, multidimensional recurrence quantification analysis parameters, estimation of the Lyapunov spectra, maximum Lyapunov exponent, or any combination thereof.

In some embodiments, recurrence quantification analysis, cross-recurrence quantification analysis, joint recurrence quantification analysis, multi-dimensional recurrence quantification analysis of the one or more exposomic biochemical signatures may be used to derive descriptive statistics and/or parameters that are utilized in to train predictive models, described elsewhere herein.

In some instances, the recurrence quantification analysis parameters may comprise recurrence rates, determinism, mean diagonal length, maximum diagonal length, divergence, Shannon entropy in diagonal length, trend in recurrences, laminarity, trapping time, maximum vertical line length, Shannon entropy in vertical line lengths, mean recurrence time, Shannon entropy in recurrence times, and number of the most probable recurrences.

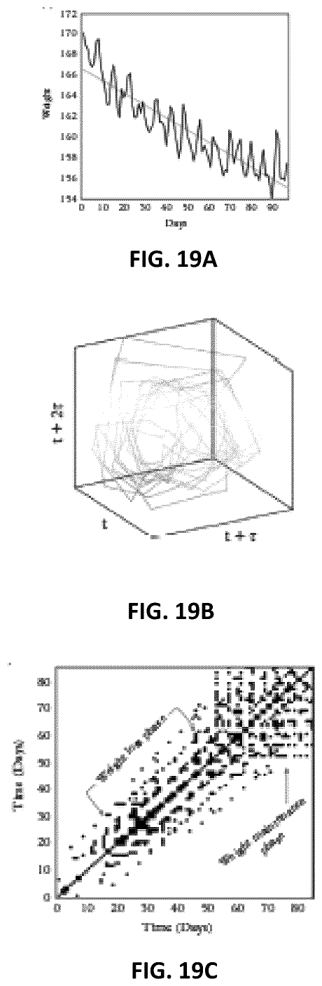

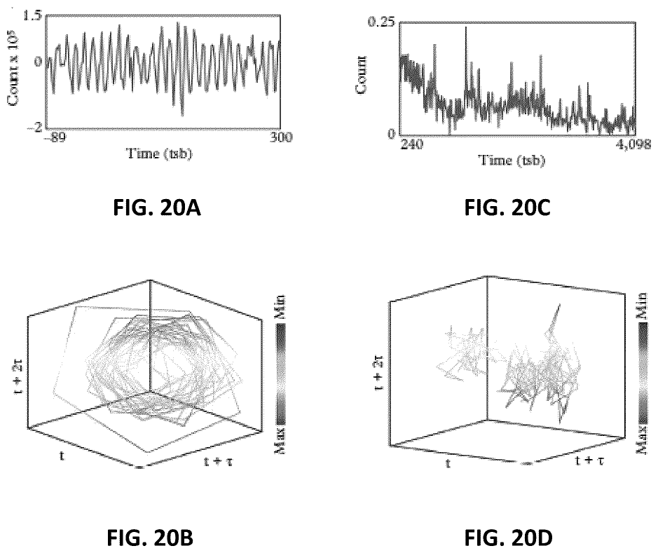

In some instances, the one or more exposomic features may be derived from one or more attractors (FIGS. 19B, 20B, 20D), whereby the one or more attractors are generated from the one or more dynamic exposomic biochemical signatures (FIG. 19A, 20A, 20C). In some embodiments, the one or more attractors are analyzed by potential energy analysis thereby producing a potential energy data space.

In some embodiments, a dynamic relationship (FIG. 19C) is established between the one or more signal recurrence rates, determinism, mean diagonal length, maximum diagonal length, divergence, Shannon entropy in diagonal length, trend in recurrences, laminarity, trapping time, maximum vertical line length, Shannon entropy in vertical line lengths, mean recurrence time, Shannon entropy in recurrence times, number of the most probable recurrences, or any combination thereof, may be analyzed and provided as a feature. In some embodiments, the dynamic relationship is determined by cross-convergent mapping (CCM).

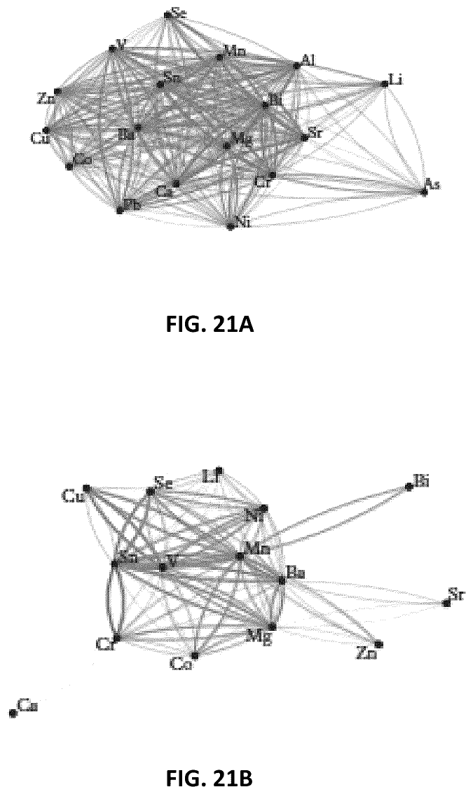

In some instances, a network may be constructed (FIGS. 21A-B) based on similarity of the one or more attractors' temporal exposomic data signal recurrence rates, determinism, mean diagonal length, maximum diagonal length, divergence, Shannon entropy in diagonal length, trend in recurrences, laminarity, trapping time, maximum vertical line length, Shannon entropy in vertical line lengths, mean recurrence time, Shannon entropy in recurrence times, number of the most probable recurrences, or any combination thereof. In some embodiments, one or more exposomic features of the network of the one or more attractors is analyzed to determine network connectivity, efficiency, feature importance, pathway importance, related graph-theory based metrics, or any combination thereof analysis.

In some embodiments, the one or more exposomic features of the one or more dynamic exposomic signatures may comprise phenotypic features. The phenotypic features may comprise: electrocardiogram (ECG), electroencephalography, magnetic resonance imaging (MRI), functional magnetic resonance imaging (fMRI), positron emission tomography (PET), genomic, epigenomic, transcriptomic, proteomic, metabolomic, or any combination thereof data.

In some embodiments, the phenotypic exposomic features comprise molecular phenotypes. In some instances, the molecular phenotypes are determined by unsupervised analysis, where the unsupervised analysis comprises clustering, dimensionality-reduction, factor analysis, stacked autoencoding, or any combination thereof.

In some embodiments, the CBD 3 may comprise clinical phenotype data for subjects. In some embodiments, clinical phenotype data comprise clinical metadata for a plurality of subjects. In some embodiments, the clinical metadata may comprise the subject's age, gender, weight, height, blood type, eye vision, current diseases, past history of family diseases, or any combination thereof. In some embodiments, the clinical metadata and exposome biochemical signature of a subject may be considered independent or in combination to determine if a subject would be a suitable candidate for an intervention.

In some embodiments, the IODB 5 may comprise intervention outcome information. In some embodiments, the intervention outcome information may comprise eligibility criterion for one or more intervention. In some embodiments, the intervention may comprise a phase 1, 2, 3 or any combination thereof intervention. In some embodiments, the intervention outcome information may comprise information for one or more subjects' intervention outcome classification comprising: positive responder, negative responder, or non-responder.

In some embodiments, the TDB 18, may comprise exposomic features that pertain at least in part to pharmaceutical and nutraceutical treatments. In some embodiments, the exposomic features of pharmaceutical and nutraceutical may comprise one or more reference exposomic features of subjects that have not participated in space travel, one or more pre-treatment exposomic features of subjects that have participated in space travel, and one or more post-treatment exposomic features of subjects that have participated in space travel obtained by assaying one or more biological sample of one or more subjects.

In some embodiments, the analysis of the differences between the one or more pre-treatment exposomic features and the one or more post-treatment exposomic features to the one or more reference dynamic profile biochemical signatures may be used to determine one or more optimal pharmaceutical, nutraceutical, or any combination thereof treatments for a subject that has participated in space travel. In some embodiments, a difference of the one or more post-treatment exposomic features towards the one or more reference exposomic features may provide the basis for recommending one or more pharmaceutical or nutraceuticals for a subject that has participated in space travel and may be used in combination with one or more subjects' exposome biochemical signature to recommend optimal treatments to ameliorate the negative effects of space travel. In some embodiments, the criterion for an optimal pharmaceutical or nutraceutical treatment may comprise a complement to the deficiencies of a given subject's exposome biochemical signatures of the one or more subjects.

In some embodiments, the difference of one or more features of the post-treatment exposomic features to the one or more reference exposomic features. In some embodiment, the feature comprises an overall mean, a measure of variability, a moving average, etc., or any combination thereof features, as described elsewhere herein.

In some embodiments, the difference of the features comprises a difference by about 10% to about 100%. In some embodiments, the difference of the features comprises a difference by about 10% to about 20%, about 10% to about 30%, about 10% to about 40%, about 10% to about 50%, about 10% to about 60%, about 10% to about 70%, about 10% to about 80%, about 10% to about 90%, about 10% to about 100%, about 20% to about 30%, about 20% to about 40%, about 20% to about 50%, about 20% to about 60%, about 20% to about 70%, about 20% to about 80%, about 20% to about 90%, about 20% to about 100%, about 30% to about 40%, about 30% to about 50%, about 30% to about 60%, about 30% to about 70%, about 30% to about 80%, about 30% to about 90%, about 30% to about 100%, about 40% to about 50%, about 40% to about 60%, about 40% to about 70%, about 40% to about 80%, about 40% to about 90%, about 40% to about 100%, about 50% to about 60%, about 50% to about 70%, about 50% to about 80%, about 50% to about 90%, about 50% to about 100%, about 60% to about 70%, about 60% to about 80%, about 60% to about 90%, about 60% to about 100%, about 70% to about 80%, about 70% to about 90%, about 70% to about 100%, about 80% to about 90%, about 80% to about 100%, or about 90% to about 100%. In some embodiments, the difference of the features may comprise a difference by about 10%, about 20%, about 30%, about 40%, about 50%, about 60%, about 70%, about 80%, about 90%, or about 100%. In some embodiments, the difference of the features may comprise a difference by at least about 10%, about 20%, about 30%, about 40%, about 50%, about 60%, about 70%, about 80%, or about 90%. In some embodiments, the difference of the features may comprise a difference by at most about 20%, about 30%, about 40%, about 50%, about 60%, about 70%, about 80%, about 90%, or about 100%.

The computer system may further comprise a communication interface 13 (e.g., network adapter) for communicating with one or more other systems, and peripheral devices 15, such as cache, other memory, data storage and/or electronic display adapters. The memory 17, storage unit 19, interface 13 and peripheral devices 15 are in communication with the CPU 21 through a communication bus (solid lines), such as a motherboard. The storage unit 19 can be a data storage unit (or data repository) for storing data. The computer system 11 can be operatively coupled to a computer network (“network”) with the aid of the communication interface 13. The network can be the Internet, an extranet, and/or an intranet that is in communication with the Internet. The network, in some embodiments, is a telecommunication and/or data network. The network can include one or more computer servers, which can enable distributed computing, such as cloud computing. The network, in some cases with the aid of the computer system 11, can implement a peer-to-peer network, which may enable devices coupled to the computer system 11 to behave as a client or a server.

In some embodiment, the EDB 1, CDB 3, IODB 5, and TDB 18 may be located on the network and accessed remotely by the computer system 11. In some embodiments, the EDB 1, CDB 3, IODB 5, and TDB 18 may reside on a secure encrypted network server that protects personal health information. In some embodiments, the EDB 1, CDB 3, IODB 5, and TDB 18, may be accessed remotely by one or more computer systems 11 within or external to a network of hospitals. In some embodiments, the EDB 1, CDB 3, IODB 5, and TDB 18 and may be accessed by one or more subjects over secure network protocols to view recommendations and their personalized data.

The CPU 21 can execute a sequence of machine-readable instructions, which can be embodied in a program or software. The instructions may be stored in a memory location, such as the random-access memory 17. The instructions can be directed to the CPU 21, which can subsequently program or otherwise configure the CPU 21 to implement methods of the present disclosure. Examples of operations performed by the CPU 21 can include fetch, decode, execute, and writeback.

The CPU 21 can be part of a circuit, such as an integrated circuit. One or more other components of the system 11 can be included in the circuit. In some embodiments, the circuit is an application specific integrated circuit (ASIC).

The storage unit 19 can store files, such as drivers, libraries, and saved programs. The storage unit 19 can store user data, e.g., user preferences and user programs. The computer system 11 in some cases can include one or more additional data storage units that are external to the computer system 11, such as located on a remote server that is in communication with the computer system 11 through an intranet or the Internet.

The computer system 11 may communicate with one or more remote computer systems through the network. For instance, the computer system 11 can communicate with a remote computer system of a user (e.g., a health care provider, subjects, etc.). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 11 via the network.

Methods as described herein can be implemented by way of machine (e.g., computer processor) executable code stored on an electronic storage location of the computer system 11, such as, for example, on the memory 17 or electronic storage unit 19. The machine executable or machine-readable code can be provided in the form of software. During use, the code can be executed by the processor 21. In some embodiments, the code is retrieved from the storage unit 19 and stored on the random-access memory 17 for ready access by the processor 21. In some situations, the storage unit 19 can be precluded, and machine-executable instructions are stored on the random-access memory 17.

The code can be pre-compiled and configured for use with a machine having a processer adapted to execute the code or can be compiled during runtime. The code can be supplied in a programming language that can be selected to enable the code to execute in a pre-compiled or as-compiled fashion.

Aspects of the systems and methods provided herein, such as the computer system 11, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical, and electromagnetic waves, such as used across physical interfaces between local devices, through wired and optical landline networks and over various air-links. The physical elements that carry such waves, such as wired or wireless links, optical links, or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, may be used to implement the databases, etc. shown in the drawings. Volatile storage media include dynamic memory, such as main memory of such a computer platform. Tangible transmission media include coaxial cables; copper wire; and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 11 can include or be in communication with an electronic display 7 that comprises a user interface (UI) 9 for providing, for example, fluorescence image data, fluorescence intensity data, temporal profiles of inflammation, and machine learning classifications. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 21, described elsewhere herein.

### Predictive Models

Aspects of the disclosure herein may comprise trained predictive models implemented on the computer implemented exposomic system 23. In some embodiments, the trained predictive models may be configured to provide retrospective or prospective predictions of subjects' probability of success for interventions, pharmaceutical or nutraceutical treatments for subjects in need thereof, or any combination thereof. In some embodiments, the trained predictive models may comprise a statistical, machine learning, artificial intelligence classifiers, an ensemble of classifiers, or any combination thereof.

The classifier may comprise one or more statistical, machine learning, or artificial intelligence algorithms. Examples of utilized algorithms may include a support vector machine (SVM), a naïve Bayes classification, a random forest, a neural network (such as a deep neural network (DNN), a recurrent neural network (RNN), a deep RNN, a long short-term memory (LSTM) recurrent neural network (RNN), or a gated recurrent unit (GRU), or other supervised learning algorithm or unsupervised machine learning, statistical, deep-learning algorithm, shallow-learning algorithm for classification and regression. The classifier may likewise involve the estimation of ensemble models, comprised of multiple predictive models, and utilize techniques such as gradient boosting, for example in the construction of gradient-boosting decision trees. The classifier may be trained using one or more training datasets corresponding to patient data. In some embodiments, the one or more training datasets may comprise exposome biochemical signatures, dynamic exposome biochemical signatures, clinical metadata, clinical trial information, exposomic features of pharmaceutical and nutraceutical treatments, or any combination thereof.

In some embodiments, training data features may comprise subjects' dynamic exposomic biochemical signature data generated from biological samples. For each biological sample of a given subject, a plurality of positions of a reference line on a biological sample of the training subject may be sampled in order to generate measurements therefrom, thereby obtaining a plurality of exposomic biochemical signatures. Each exposomic biochemical signature in the corresponding plurality of exposomic biochemical signatures corresponds to a different position in the corresponding plurality of positions, and each position in the corresponding plurality of positions represents a different period of growth of the corresponding biological sample. Next, each respective position of the biological sample is analyzed (e.g., using a laser ablation-inductively coupled-plasma mass spectrometer (LA-ICP-MS), a fluorescence image sensor, or a Raman spectrometer) to obtain a plurality of traces. Each trace in the corresponding plurality of traces corresponds to an abundance measurement of a corresponding substance, which are over time collectively determined from the corresponding plurality of dynamic exposome biochemical signatures.

In some embodiments, labels may comprise intervention outcomes such as, for example, a positive response, negative response (i.e., adverse response), or a non-responder. Intervention outcomes may comprise a temporal characteristic associated with the classification of a positive response, negative response, or non-responder event to the duration of time after administration of the treatment provided during intervention. Such a period of time may be, for example, about 1 hour, about 2 hours, about 3 hours, about 4 hours, about 6 hours, about 8 hours, about 10 hours, about 12 hours, about 14 hours, about 16 hours, about 18 hours, about 20 hours, about 22 hours, about 24 hours, about 2 days, about 3 days, about 4 days, about 5 days, about 6 days, about 7 days, about 10 days, about 2 weeks, about 3 weeks, about 4 weeks, about 1 month, about 2 months, about 3 months, about 4 months, about 6 months, about 8 months, about 10 months, about 1 year, or more than about 1 year.

Input features for training the classifier may be structured by aggregating the data into bins or alternatively using one-hot encoding. Inputs may also include feature values or vectors derived from the previously mentioned inputs, such as cross-correlations calculated between separate exposomic features or other measurements over a fixed period of time, and the discrete derivative or the finite difference between successive measurements, described elsewhere herein. Such a period of time may be, for example, about 1 hour, about 2 hours, about 3 hours, about 4 hours, about 6 hours, about 8 hours, about 10 hours, about 12 hours, about 14 hours, about 16 hours, about 18 hours, about 20 hours, about 22 hours, about 24 hours, about 2 days, about 3 days, about 4 days, about 5 days, about 6 days, about 7 days, about 10 days, about 2 weeks, about 3 weeks, about 4 weeks, about 1 month, about 2 months, about 3 months, about 4 months, about 6 months, about 8 months, about 10 months, about 1 year, or more than about 1 year.

Training records may be constructed from sequences of observations. Such sequences may comprise a fixed length for ease of data processing. For example, sequences may be zero-padded or selected as independent subsets of a single patient's records

In order to train the classifier model (e.g., by determining weights and correlations of the model) to generate real-time classifications or predictions, the model can be trained using datasets. Such datasets may be sufficiently large to generate statistically significant classifications or predictions. For example, datasets may comprise: databases of de-identified data including dynamic profile biological signature data and other clinical metadata measurements from a hospital or other clinical setting.

Datasets may be split into subsets (e.g., discrete or overlapping), such as a training dataset, a development dataset, and a test dataset. For example, a dataset may be split into a training dataset comprising 80% of the dataset and a test dataset comprising 20% of the dataset. The training dataset may comprise about 10%, about 20%, about 30%, about 40%, about 50%, about 60%, about 70%, about 80%, or about 90% of the dataset. The development dataset may comprise about 10%, about 20%, about 30%, about 40%, about 50%, about 60%, about 70%, about 80%, or about 90% of the dataset. The test dataset may comprise about 10%, about 20%, about 30%, about 40%, about 50%, about 60%, about 70%, about 80%, or about 90% of the dataset. Training sets (e.g., training datasets) may be selected by random sampling of a set of data corresponding to one or more subject cohorts to ensure independence of sampling.

Alternatively, training sets (e.g., training datasets) may be selected by proportionate sampling of a set of data corresponding to one or more subject cohorts to ensure independence of sampling.

To improve the accuracy of model predictions and reduce overfitting of the model, the datasets may be augmented to increase the number of samples within the training set. For example, data augmentation may comprise rearranging the order of observations in a training record. To accommodate datasets having missing observations, methods to impute missing data may be used, such as forward-filling, back-filling, linear interpolation, and multi-task Gaussian processes. Datasets may be filtered to remove confounding factors. For example, within a database, a subset of subjects may be excluded.

In some embodiments, data science techniques, such as dropout or regularization, may be used during training the classifier to prevent overfitting. The neural network may comprise a plurality of sub-networks, each of which is configured to generate a classification or prediction of a different type of output information (e.g., which may be combined to form an overall output of the neural network). The classifier may alternatively utilize statistical or related algorithms including random forest, classification and regression trees, support vector machines, discriminant analyses, regression techniques, as well as ensemble and gradient-boosted variations thereof.

In some embodiments, the systems and methods of the present disclosure are deployed in a hospital setting for patients that are active receiving treatment to ameliorate the negative effects of space travel. When the classifier generates a classification or a prediction for an optimal pharmaceutical or nutraceutical, a notification (e.g., alert or alarm) may be generated and transmitted to a health care provider, such as a physician, nurse, or other member of the patient's treating team within a hospital. Notifications may be transmitted via an automated phone call, a short message service (SMS) or multimedia message service (MMS) message, an e-mail, an alert within a dashboard, or any combination thereof. The notification may comprise output information such as a prediction for the outcome of an intervention or an optimal pharmaceutical or nutraceutical.

To validate the performance of the classifier model, different performance metrics may be generated. For example, an area under the receiver-operating curve (AUROC) may be used to determine the predictive capability of the classifier. For example, the classifier may use classification thresholds which are adjustable, such that specificity and sensitivity are tunable, and the receiver-operating curve (ROC) can be used to identify the different operating points corresponding to different values of specificity and sensitivity.

In some embodiments, the performance of predictive methods is assessed by constructing tables to provide the frequency and overlap of predicted cases of individuals with one or more exposomic signatures and/or features indicative of space travel and actual positive cases, predicted positive cases and actual negative cases, predicted negative cases and actual negative cases, and/or predicted negative cases and actual positive cases. In some instances, the tables constructed may be confusion matrices. In some cases, cross-tabulation of the confusion matrices may provide sensitivity, specificity, accuracy, and related performance metrics associated with systems and methods described elsewhere herein, at a given predictive threshold.

In some embodiments, such as when datasets are not sufficiently large, cross-validation may be performed to assess the robustness of a classifier model across different training and testing datasets.

To calculate performance metrics such as sensitivity, specificity, accuracy, positive predictive value (PPV), negative predictive value (NPV), AUPRC, AUROC, or similar, the following definitions may be used. A “false positive” may refer to an outcome in which a positive outcome or result has been incorrectly generated (e.g., a subject classified as a positive responder to an intervention, yet they experience an adverse or negative effect of participating in the intervention). A “true positive” may refer to an outcome in which positive outcome or result has been correctly generated (e.g., the subject is classified as a positive responder to an intervention, and they experience a positive response). A “false negative” may refer to an outcome in which a negative outcome or result has been generated (e.g., the subject is classified as a negative responder where the subject after participating in the intervention is a non-responder or a positive responder). A “true negative” may refer to an outcome in which a negative outcome or result has been generated (e.g., the subject is classified as a negative responder and after participating in the intervention the subject responds adversely to the pharmaceutical treatment of the intervention).

In some embodiments, the classifier may be trained until certain pre-determined conditions for accuracy or performance are satisfied, such as having minimum desired values corresponding to predictive accuracy measures. For example, the predictive accuracy measure may correspond to correct prediction for an outcome of an intervention or an optimal pharmaceutical or nutraceutical recommendation and/or selection. Examples of diagnostic accuracy measures may include sensitivity, specificity, positive predictive value (PPV), negative predictive value (NPV), accuracy, area under the precision-recall curve (AUPRC), and area under the curve (AUC) of a Receiver Operating Characteristic (ROC) curve (AUROC) corresponding to the diagnostic accuracy of detecting determining whether or not a subject has participated in space travel and/or the subjects exposomic signatures and/or features resemble that of a subject that has participated in space travel.

For example, such a pre-determined condition may be that the sensitivity of predicting the outcome of an intervention or an optimal pharmaceutical or nutraceutical recommendation and/or selection comprises a value of, for example, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99%.

As another example, such a pre-determined condition may be that the specificity of predicting the outcome of an intervention or an optimal pharmaceutical or nutraceutical recommendation and/or selection comprises a value of, for example, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99%.

As another example, such a pre-determined condition may be that the positive predictive value (PPV) of predicting the outcome of an intervention or an optimal pharmaceutical or nutraceutical recommendation and/or selection comprises a value of, for example, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99%.

As another example, such a pre-determined condition may be that the negative predictive value (NPV) of outcome of an intervention or an optimal pharmaceutical or nutraceutical recommendation and/or selection comprises a value of, for example, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99%.

As another example, such a pre-determined condition may be that the area under the curve (AUC) of a Receiver Operating Characteristic (ROC) curve (AUROC) of predicting the outcome of an intervention or an optimal pharmaceutical or nutraceutical recommendation and/or selection comprises a value of at least about 0.50, at least about 0.55, at least about 0.60, at least about 0.65, at least about 0.70, at least about 0.75, at least about 0.80, at least about 0.85, at least about 0.90, at least about 0.95, at least about 0.96, at least about 0.97, at least about 0.98, or at least about 0.99.

As another example, such a pre-determined condition may be that the area under the precision-recall curve (AUPRC) of predicting the outcome of an intervention or an optimal pharmaceutical or nutraceutical recommendation and/or selection comprises a value of at least about 0.10, at least about 0.15, at least about 0.20, at least about 0.25, at least about 0.30, at least about 0.35, at least about 0.40, at least about 0.45, at least about 0.50, at least about 0.55, at least about 0.60, at least about 0.65, at least about 0.70, at least about 0.75, at least about 0.80, at least about 0.85, at least about 0.90, at least about 0.95, at least about 0.96, at least about 0.97, at least about 0.98, or at least about 0.99.

In some embodiments, the trained classifier may be trained or configured to predict the outcome of an intervention or an optimal pharmaceutical or nutraceutical recommendation and/or selection with a sensitivity of at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99%.

In some embodiments, the trained classifier may be trained or configured to predict the outcome of an intervention or an optimal pharmaceutical or nutraceutical recommendation and/or selection with a specificity of at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99%.

In some embodiments, the trained classifier may be trained or configured to predict the outcome of an intervention or an optimal pharmaceutical or nutraceutical recommendation and/or selection with a positive predictive value (PPV) of at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99%.

In some embodiments, the trained classifier may be trained or configured to predict the outcome of an intervention or an optimal pharmaceutical or nutraceutical recommendation and/or selection with a negative predictive value (NPV) of at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99%.

In some embodiments, the trained classifier may be trained or configured to predict the outcome of an intervention or an optimal pharmaceutical or nutraceutical recommendation and/or selection with an area under the curve (AUC) of a Receiver Operating Characteristic (ROC) curve (AUROC) of at least about 0.50, at least about 0.55, at least about 0.60, at least about 0.65, at least about 0.70, at least about 0.75, at least about 0.80, at least about 0.85, at least about 0.90, at least about 0.95, at least about 0.96, at least about 0.97, at least about 0.98, or at least about 0.99.

In some embodiments, the trained classifier may be trained or configured to predict the outcome of an intervention or an optimal pharmaceutical or nutraceutical recommendation and/or selection with an area under the precision-recall curve (AUPRC) of at least about 0.10, at least about 0.15, at least about 0.20, at least about 0.25, at least about 0.30, at least about 0.35, at least about 0.40, at least about 0.45, at least about 0.50, at least about 0.55, at least about 0.60, at least about 0.65, at least about 0.70, at least about 0.75, at least about 0.80, at least about 0.85, at least about 0.90, at least about 0.95, at least about 0.96, at least about 0.97, at least about 0.98, or at least about 0.99.

In some embodiments, the classifier is a neural network or a convolutional neural network. See, Vincent et al., 2010, “Stacked denoising autoencoders: Learning useful representations in a deep network with a local denoising criterion,” J Mach Learn Res 11, pp. 3371-3408; Larochelle et al., 2009, “Exploring strategies for training deep neural networks,” J Mach Learn Res 10, pp. 1-40; and Hassoun, 1995, Fundamentals of Artificial Neural Networks, Massachusetts Institute of Technology, each of which is hereby incorporated by reference.

SVMs are described in Cristianini and Shawe-Taylor, 2000, “An Introduction to Support Vector Machines,” Cambridge University Press, Cambridge; Boser et al., 1992, “A training algorithm for optimal margin classifiers,” in Proceedings of the 5th Annual ACM Workshop on Computational Learning Theory, ACM Press, Pittsburgh, Pa., pp. 142-152; Vapnik, 1998, Statistical Learning Theory, Wiley, New York; Mount, 2001, Bioinformatics: sequence and genome analysis, Cold Spring Harbor Laboratory Press, Cold Spring Harbor, N.Y.; Duda, Pattern Classification, Second Edition, 2001, John Wiley & Sons, Inc., pp. 259, 262-265; and Hastie, 2001, The Elements of Statistical Learning, Springer, New York; and Furey et al., 2000, Bioinformatics 16, 906-914, each of which is hereby incorporated by reference in its entirety. When used for classification, SVMs separate a given set of binary labeled data with a hyper-plane that is maximally distant from the labeled data. For cases in which no linear separation is possible, SVMs can work in combination with the technique of ‘kernels’, which automatically realizes a non-linear mapping to a feature space. The hyper-plane found by the SVM in feature space corresponds to a non-linear decision boundary in the input space.

Decision trees are described generally by Duda, 2001, Pattern Classification, John Wiley & Sons, Inc., New York, pp. 395-396, which is hereby incorporated by reference. Tree-based methods partition the feature space into a set of rectangles, and then fit a model (like a constant) in each one. In some embodiments, the decision tree is random forest regression. One specific algorithm that can be used is a classification and regression tree (CART). Other specific decision tree algorithms include, but are not limited to, ID3, C4.5, MART, and Random Forests. CART, ID3, and C4.5 are described in Duda, 2001, Pattern Classification, John Wiley & Sons, Inc., New York. pp. 396-408 and pp. 411-412, which is hereby incorporated by reference. CART, MART, and C4.5 are described in Hastie et al., 2001, The Elements of Statistical Learning, Springer-Verlag, New York, Chapter 9, which is hereby incorporated by reference in its entirety. Random Forests are described in Breiman, 1999, “Random Forests-Random Features,” Technical Report 567, Statistics Department, U.C. Berkeley, September 1999, which is hereby incorporated by reference in its entirety.

Clustering (e.g., unsupervised clustering model algorithms and supervised clustering model algorithms) is described at pages 211-256 of Duda and Hart, Pattern Classification and Scene Analysis, 1973, John Wiley & Sons, Inc., New York, (hereinafter “Duda 1973”) which is hereby incorporated by reference in its entirety. As described in Section 6.7 of Duda 1973, the clustering problem is described as one of finding natural groupings in a dataset. To identify natural groupings, two issues are addressed. First, a way to measure similarity (or dissimilarity) between two samples is determined. This metric (similarity measure) is used to ensure that the samples in one cluster are more like one another than they are to samples in other clusters. Second, a mechanism for partitioning the data into clusters using the similarity measure is determined. Similarity measures are discussed in Section 6.7 of Duda 1973, where it is stated that one way to begin a clustering investigation is to define a distance function and to compute the matrix of distances between all pairs of samples in the training set. If distance is a good measure of similarity, then the distance between reference entities in the same cluster will be significantly less than the distance between the reference entities in different clusters. However, as stated on page 215 of Duda 1973, clustering does not require the use of a distance metric. For example, a nonmetric similarity function s(x, x′) can be used to compare two vectors x and x′. Conventionally, s(x, x′) is a symmetric function whose value is large when x and x′ are somehow “similar.” An example of a nonmetric similarity function s(x, x′) is provided on page 218 of Duda 1973. Once a method for measuring “similarity” or “dissimilarity” between points in a dataset has been selected, clustering requires a criterion function that measures the clustering quality of any partition of the data. Partitions of the data set that extremize the criterion function are used to cluster the data. See page 217 of Duda 1973. Criterion functions are discussed in Section 6.8 of Duda 1973. More recently, Duda et al., Pattern Classification, 2nd edition, John Wiley & Sons, Inc. New York, has been published. Pages 537-563 describe clustering in detail. More information on clustering techniques can be found in Kaufman and Rousseeuw, 1990, Finding Groups in Data: An Introduction to Cluster Analysis, Wiley, New York, N.Y.; Everitt, 1993, Cluster analysis (3d ed.), Wiley, New York, N.Y.; and Backer, 1995, Computer-Assisted Reasoning in Cluster Analysis, Prentice Hall, Upper Saddle River, New Jersey, each of which is hereby incorporated by reference. Particular exemplary clustering techniques that can be used in the present disclosure include, but are not limited to, hierarchical clustering (agglomerative clustering using nearest-neighbor algorithm, farthest-neighbor algorithm, the average linkage algorithm, the centroid algorithm, or the sum-of-squares algorithm), k-means clustering, fuzzy k-means clustering algorithm, and Jarvis-Patrick clustering. In some embodiments, the clustering comprises unsupervised clustering, where no preconceived notion of what clusters should form when the training set is clustered, are imposed.

Regression models, such as that of the multi-category logit models, are described in Agresti, An Introduction to Categorical Data Analysis, 1996, John Wiley & Sons, Inc., New York, Chapter 8, which is hereby incorporated by reference in its entirety. In some embodiments, the classifier makes use of a regression model disclosed in Hastie et al., 2001, The Elements of Statistical Learning, Springer-Verlag, New York, which is hereby incorporated by reference in its entirety. In some embodiments, gradient-boosting models are used toward, for example, the classification algorithms described herein; these gradient-boosting models are described in Boehmke, Bradley; Greenwell, Brandon (2019). “Gradient Boosting”. Hands-On Machine Learning with R. Chapman & Hall. pp. 221-245. ISBN 978-1-138-49568-5., which is hereby incorporated by reference in its entirety. In some embodiments, ensemble modeling techniques are used, for example, toward the classification algorithms described herein; these ensemble modeling techniques are described in the implementation of classification models herein, are described in Zhou Zhihua (2012). Ensemble Methods: Foundations and Algorithms. Chapman and Hall/CRC. ISBN 978-1-439-83003-1, which is hereby incorporated by reference in its entirety.

In some embodiments, the machine learning analysis is performed by a device executing one or more programs (e.g., one or more programs stored in the Non-Persistent Memory (i.e., RAM or ROM) 17 or in the storage unit 19 (i.e., hard-disk) in FIG. 1 including instructions to perform the data analysis. In some embodiments, the data analysis is performed by a system comprising at least one processor (e.g., the processing core 21) and memory (e.g., one or more programs stored in the Non-Persistent Memory 17 or in the storage unit 19) comprising instructions to perform the data analysis

### Intervention Stratification Predictive Models

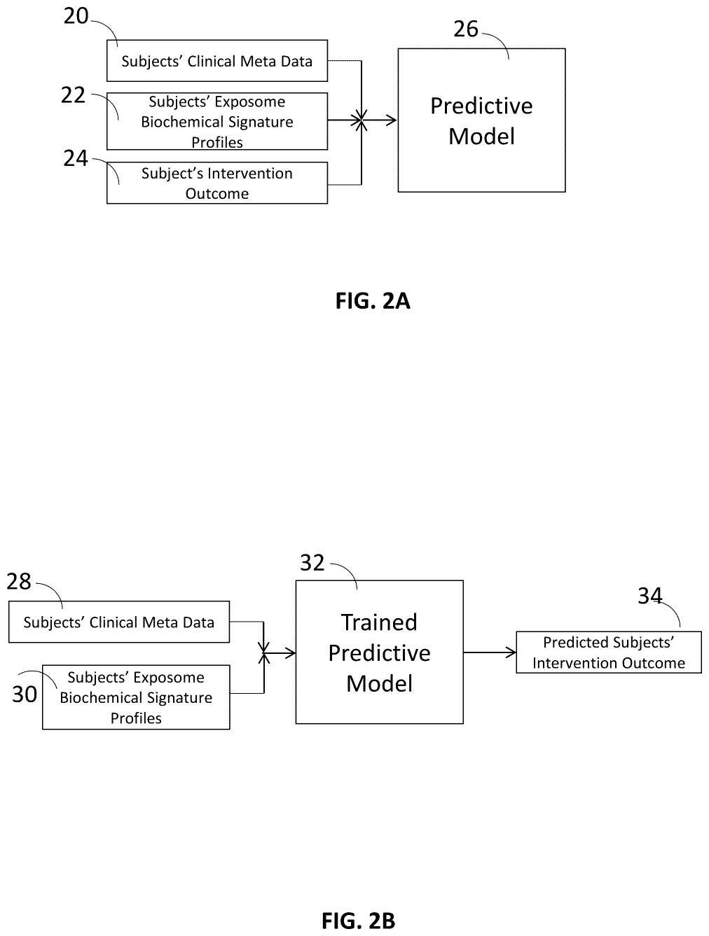

In some embodiments, the predictive model 26 may comprise one or more classifiers that may be trained to predict the probability of intervention outcomes for one or more subjects based on their exposomic features, as seen in FIG. 2A. In some embodiments, the inputs for training the classifier may comprise subjects' clinical meta data 20, subjects' exposomic biochemical signature data 22, and the subjects' corresponding intervention outcome 24. In some embodiments, the intervention outcome for a given subject may comprise, non-responder, adverse responder, or positive responder. In some embodiments, the predictive model may be initially trained on a dataset of one or more subjects having undergone treatment from the intervention. In some embodiments, training data sets used for training a classifier may be generated from, for example, the subjects' clinical meta data and the corresponding subjects' exposomic biochemical signature profiles, or features derived from the exposomic biochemical signature profiles, for example via RQA, described elsewhere herein, and intervention outcomes.

In some embodiments, clinical metadata features may comprise subjects' demographic information derived from electronic medical records (EMR), physiological measurements, and intervention outcomes. Additionally, training features may comprise clinical characteristics such as, for example, certain ranges or categories of dynamic exposome biochemical signature data. For example, a set of features collected from a given patient at a given time point may collectively serve as a signature of the CBD 3 and EDB 1, which may be indicative of a health state or status of the subjects.

In some embodiments, the trained predictive model 32 may comprise a trained classifier, described elsewhere herein, configured to provide predictions of the intervention outcome with regards to subjects interested in participating in an intervention, as seen in FIG. 2B. In some embodiments, one or more subjects' clinical metadata 28 and corresponding exposomic features 30 may be fed as an input into the trained predictive model 32. The trained predictive model 32 may then output a probability of a predicted subjects' trial outcome 34. In some embodiments, the output probability of predicted subjects' trial outcome may comprise a classification of a positive responder, negative or adverse responder, or non-responder. In some embodiments, the subjects' clinical meta data may comprise clinical metadata e.g., subjects' age, gender, weight, height, blood type, eye vision, current diseases, past history of family diseases, or any combination thereof. Various machine learning techniques may be cascaded such that the output of a machine learning technique may also be used as input features to subsequent layers or subsections of the classifier.

### Optimal Pharmaceutical or Nutraceutical Selection Predictive Models

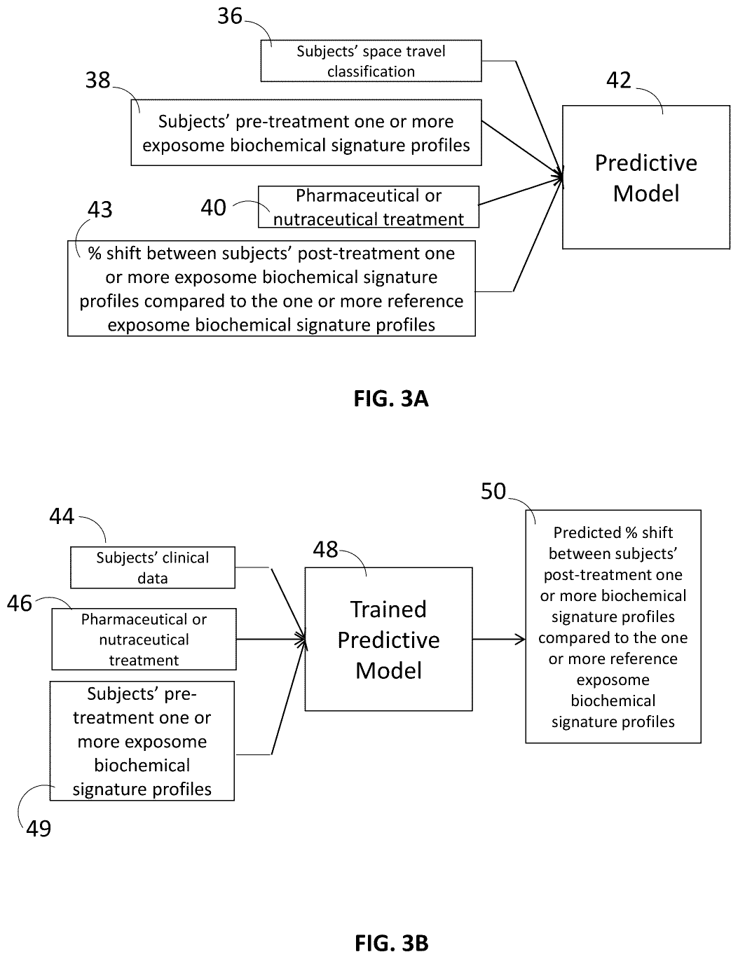

In some embodiments, the predictive model 42 may comprise one or more classifiers, described elsewhere herein, that may be trained to produce a trained predictive model 48 configured to predict the optimal pharmaceutical or nutraceutical to administered for one or more subjects that are participating in space travel based on features derived from their respective one or more exposomic features, as seen in FIG. 3A, described elsewhere herein. In some embodiments, the inputs for training statistical, machine learning, and/or artificial intelligence classifiers may comprise (a) subjects' space travel classification 36; (b) subjects' pre-treatment features derived from one or more exposomic features 38; (c) the pharmaceutical or nutraceutical treatment administered 40; and (d) the percent difference between the subjects' post-treatment features in one or more exposomic features compared to the one or more reference features derived from exposomic features of the one or more features derived from biochemical signatures of subjects that have not participated in space travel.

In some embodiments, the trained predictive model 48 may comprise a trained classifier, described elsewhere herein, configured to provide prediction 50 of the percent difference between subject's post-treatment one or more exposomic features and the one or more reference exposomic features of the one or, as seen in FIG. 3B. In some embodiments, the trained predictive model may take as inputs: (a) the subjects' clinical data 44; (b) the pharmaceutical or nutraceutical treatment under consideration 46; and (c) the subjects' pre-treatment one or more exposomic features of 49. In some embodiments, one or more pharmaceutical or nutraceutical treatments may be considered.

### Exposome Cluster Analysis

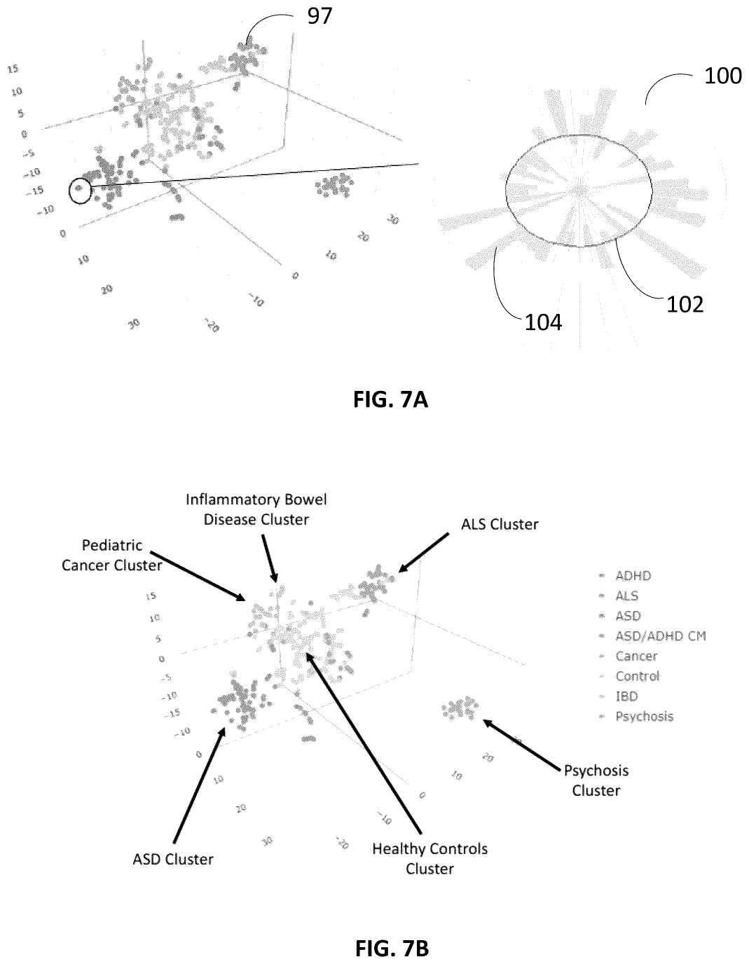

In some embodiments, one or more subjects' one or more exposome signature profiles may be analyzed by clustering methods to categorically classify or group subjects based on whether or not they have participated in space travel as seen in FIG. 7A-7B. One or more subjects are represented by exposome data clusters 97, 100 of one or more exposomic features 104. Subjects' one or more exposomic features may be compared to an average 102 of a cohort for analysis or classification. Alternatively, exposomic features may be used to sub-type subjects' prior to, during or after clinical intervention to understand which subjects may respond positively, negative, or have no response to a given intervention.

### Methods

Aspects of the disclosure herein may comprise methods of intervention optimization and recommendation of optimal pharmaceutical and or nutraceutical recommendations for subjects to ameliorate the negative effects of space travel. In some embodiments, the methods described herein may be performed on the systems of the present disclosure described elsewhere herein.

### Intervention Optimization

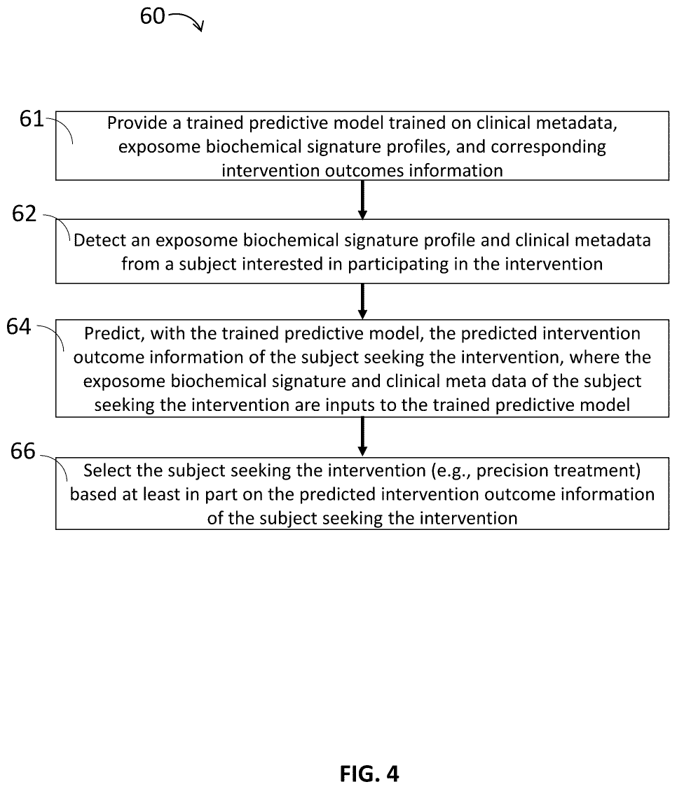

In some embodiments, the methods of the disclosure may comprise a method of optimizing the outcome of intervention for subjects 60, as seen in FIG. 4. In some embodiments, intervention may comprise clinical trial studies at phase I, phase II, phase III, or any combination thereof intervention. In some embodiments, the method comprises the steps of: (a) providing a trained predictive model, where the trained predictive model is trained on one or more subjects' clinical metadata, exposomic features, and corresponding intervention outcome information 61; (b) detecting features derived from a biochemical signature obtained from a biological sample from a subject seeking the intervention, thereby producing a retrospective exposome biochemical signature 62; (c) predicting, with the trained predictive model, the predicted intervention outcome information of the subject seeking the intervention, where the retrospective exposome biochemical signature profile and clinical meta of the subject seeking the intervention are inputs to the trained predictive model 64; (d) selecting the subject for the intervention or excluding the subject from the intervention, based at least in part on the predicted intervention outcome information of the subject 66. Alternatively, the intervention may comprise a community trial that may or may not be performed in a clinical setting.

In some instances, subjects' one or more exposomic features may be used to determine the effectiveness or efficacy of a given intervention. For example, as seen in FIGS. 6A-D, exposomic analysis of subjects' biological samples to generate exposomic features may provide insight into the effectiveness of a lead-based poisoning intervention. FIG. 6A shows an exposome biochemical signature 81 with the x-axis of days and y-axis of exposome signature intensity. FIG. 6A represents the exposome signature of tin with start 77 and end 79 points of the intervention indicated. FIG. 6B-D show contrasting exposome biochemical signatures for exposome signatures of lead, calcium, and magnesium, respectively, for one or more subjects that did not receive the intervention 85, 89, and 93 compared to subjects that did receive the intervention 83, 87, and 91. For this particular example, the exposome biochemical signature for lead in FIG. 6C may be observed to decrease, indicating that the intervention may have proven to be effective. However, it may also be observed that other exposome signatures such as magnesium increase potentially leading to unwanted effects. In some embodiments, such exposomic features of the biochemical signature are utilized to observe the effectiveness of an intervention, or to recommend adjunctive intervention to supplement indications of unwanted increases or decreases in one or more exposomic features that are not the target of the intervention. In some embodiments, such an approach of intervention efficacy or effectiveness analysis is used to repurpose interventions for non-intended applications or to aid in symptoms from space travel that the intervention was not initially intended for.

In some embodiments, exposomic features acquired from subjects receiving interventions are further analyzed with a plurality of analytical modules. In some embodiments, the first analytical module (Module 1) focuses on the effect of clinical intervention on elemental signal intensities where the time-course of intervention is established relative to the timing of exposome biochemical signature signal intensities. Time-varying signal intensities for exposome biochemical signatures may be dated to the timing of clinical intervention, allowing exposome biochemical signature signal intensities to be delineated as occurring prior to intervention, concurrent to intervention, or following an intervention. Exposome biochemical signature signal intensities during these periods can be aggregated at the level of the subject via summary statistics such as the mean or median exposome biochemical signature signal intensity detected during that period. The effect of intervention can then be assessed across subjects participating in a study through the application of traditional general linear models, where exposome biochemical signature signal intensities prior to and following intervention are compared across all subjects in order to identify statistically significant differences in exposome biochemical signature signal intensity corresponding to the effects of intervention. In this context statistical significance is evaluated through standard probabilistic hypothesis testing.

In some embodiments, the second analytical module (Module 2) may comprise a focus on the simultaneous effects of an intervention on multiple exposomic features i.e., biochemical signature pathways. As in Module 1, this module may be applied when the time-course of intervention is established relative to the timing of exposome biochemical signature signal intensities, thereby allowing the aggregation of pre-intervention, intervention-concurrent, and post-intervention intensities with descriptive statistics. Aggregate measures derived at the level of the individual are then pooled across participants in the clinical trial and used in the construction of multivariate models. These may take the form of unsupervised analyses, such as principal component analysis, factor analysis, or related methods, whereby a dimensionality-reduction technique is applied to derive metrics (principal components; factor scores) which summarize exposome biochemical signature signal intensities for multiple exposome biochemical signature pathways, which can then be used in subsequent general linear models to test hypotheses relating to clinical intervention, as in Module 1. Alternatively, a supervised-dimensionality reduction technique, such as partial least squares, partial least squares discriminant analysis, linear discriminant analysis, weighted quantile sum regression, or Bayesian kernel machine regression may be used to directly link the effect of intervention to changes in exposome biochemical signature signal intensities across multiple exposome biochemical signatures.

In some embodiments, a third analytical module (Module 3) is used in circumstances when the exact timing of an intervention is unknown, or in embodiments where the effect of the intervention is expected to have a time-lagged effect; for example, if changes in exposome biochemical signature signal intensities do not manifest for some time after treatment, or if the timing of treatment-evoked change varies among individuals. In these cases, modeling strategies derived from econometrics may be used; in particular, the implementation of distributed lag models and related non-linear methods. These methods may be extended, as in Module 2, to include the simultaneous evaluation of intervention effects in multiple exposome biochemical signatures, for example through the implementation of lagged weighted quantile sum regression. Alternative analytical strategies may comprise the use of change-point detection methods via moving average methods, self-exciting threshold autoregressive (SETAR) models, autoregressive moving average models (ARMA), bayesian change-point detection, and related methodologies, particularly relating to longitudinal modeling and change-point detection.

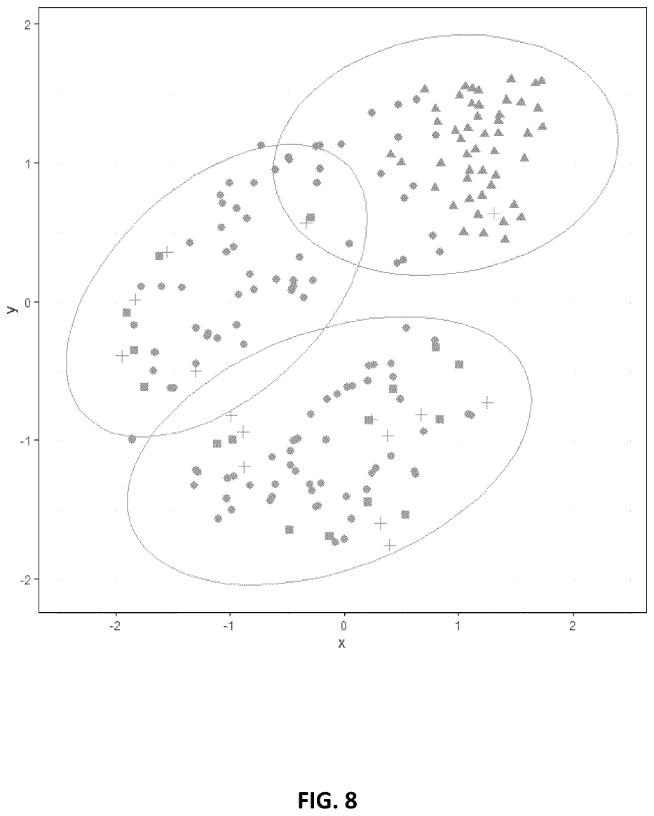

In some embodiments, a fourth analytical module (Module 4), unlike the prior models, may focus on the analysis of signal dynamics derived from analysis of longitudinal biochemical signature profile signals. Signal dynamics in this context refers to parameters derived from the analysis of biochemical signature profile signal intensities, which may include estimation of parameters descriptive of underlying processes such as periodicity, entropy, and stationarity. One approach to achieve this may comprise the application of recurrence quantification analysis (RQA) to individual biochemical signature profile signals measured in each subject trial participant. For each longitudinal measurement of a given biochemical signature profile pathway, the application of RQA may yield multiple quantitative measures or features, including any combination of recurrence rates, determinism, mean diagonal length, maximum diagonal length, divergence, Shannon entropy in diagonal length, trend in recurrences, laminarity, trapping time, maximum vertical line length, Shannon entropy in vertical line lengths, mean recurrence time, Shannon entropy in recurrence times, and number of the most probable recurrences. These features may be extracted from multiple biochemical signature pathways, and from the analysis of interactions among pathways via cross recurrence quantification analysis (CRQA), for use in subsequent analysis, where features derived from each subject are pooled and used to test for study-wide effects relating to the intervention. This approach may be particularly applicable to case-control study designs where some subjects received placebo treatment, and the interest is in distinguishing differences in elemental signal dynamics between intervention and placebo experimental conditions. In such contexts, the parameters derived from RQA/CRQA may be tested in a traditional analytical framework, for example via general linear models, in order to evaluate difference in signal parameters between treatment conditions. This approach may also be applicable to cases similar to those described in modules 1 and 2, where the time-course of a given treatment is known and the interest is in distinguishing pre-treatment, treatment-concurrent, and post-treatment conditions. In this context RQA/CRQA may be applied either to subsets of the full elemental trace, corresponding to pre-treatment, treatment-concurrent, and post-treatment conditions; or, a variant of RQA/CRQA, utilizing a windowed binning technique, may be utilized to derive a longitudinal measure of RQA/CRQA parameters, which may subsequently be analyzed with methods described in Module 1, 2, or 3. In any combination of these conditions, the features derived from dynamical signal analysis via RQA and related methods can also be used in supervised and unsupervised dimensionality reduction techniques, as described in Module 2, for the sub-typing of subjects on the basis of biochemical signature profiles. These approaches can be used prior to interventions, towards the goal of identifying patient/participant subtypes, or can be used subsequent to treatment, in order to link the effect of clinical intervention to associated metabolic pathways. As can be seen in FIG. 8, hair samples provided by subjects' with autism spectrum disorder (ASD) were analyzed via this method. The resulting projection illustrates the derivation of three ASD sub-types from the analysis of biochemical signature profiles—in this case, by application of k-means clustering to RQA of elemental profiles. The specification of subject type can thereafter be used in subsequent clinical analyses and decision-making.

### Pharmaceutical and Nutraceutical Recommendation

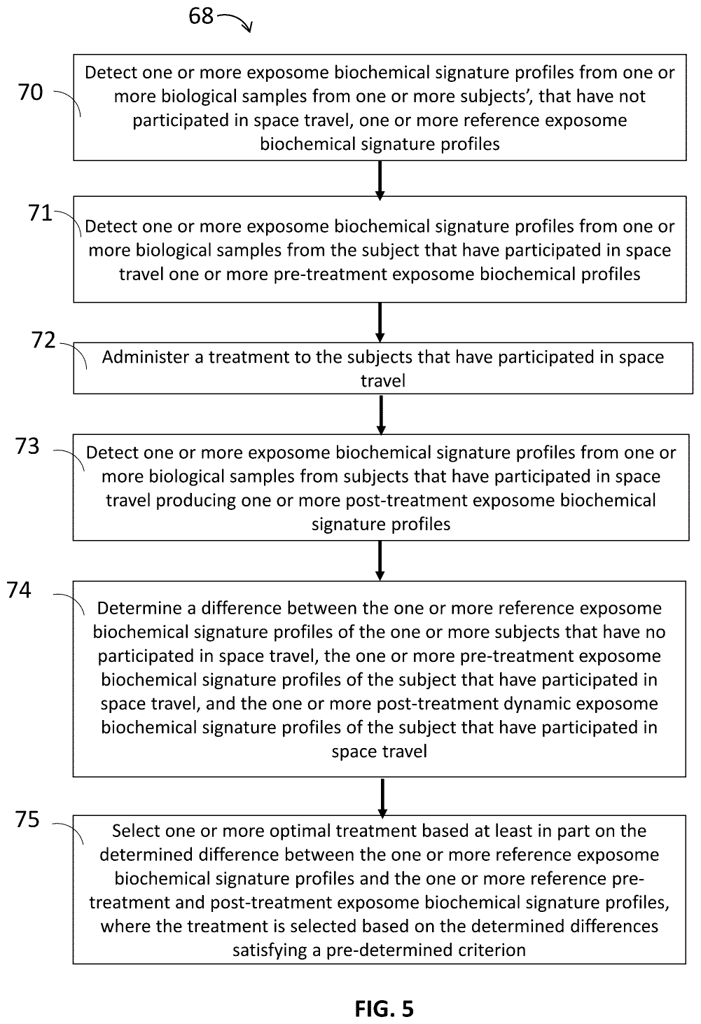

In some embodiments, the methods of the disclosure may comprise a method of optimal pharmaceutical and or nutraceutical recommendations for subjects participating in space travel 68, as seen in FIG. 5. In some embodiments, the method may comprise the steps of: (a) detecting features derived from one or more biochemical signatures obtained from one or more biological sample from one or more subjects that have not participated in space travel thereby producing a one or more reference exposomic features 70; (b) detecting features derived from one or more biochemical signature obtained from one or more biological sample from one or more subjects that have participated in space travel, thereby producing one or more features of pre-treatment exposomic features 71; (c) administering a treatment to the one or more subjects that have participated in space travel 72; (d) detecting one or more exposomic features obtained from one or more biological samples of the one or more subjects that have participated in space travel after a period of time has elapsed after receiving the treatment, thereby producing one or more post-treatment exposomic features 73; (e) determining a difference between the one or more features of the reference exposomic features of the one or more subjects that have not participated in space travel, features of the one or more pre-treatment exposomic features of the one or more subjects that have participated in space travel, and features of the one or more post-treatment exposomic features of the one or more subjects that have participated in space travel 74; and (f) selecting one or more optimal treatments based at least in part on the determined difference between the features of one or more reference exposomic features, one or more pre-treatment exposome exposomic features and one or more post-treatment exposomic features, where the one or more optimal treatments are selected based on the determined differences satisfying a pre-determined criterion 75.

In some embodiments, determining the difference between the one or more reference exposomic features of the one or more subjects that have not participated in space travel, the one or more pre-treatment exposomic features of the one or more subjects that have participated in space travel, and the one or more post-treatment exposomic features of the one or more subjects that have participated in space travel may be implemented by means of predicative models described elsewhere herein.

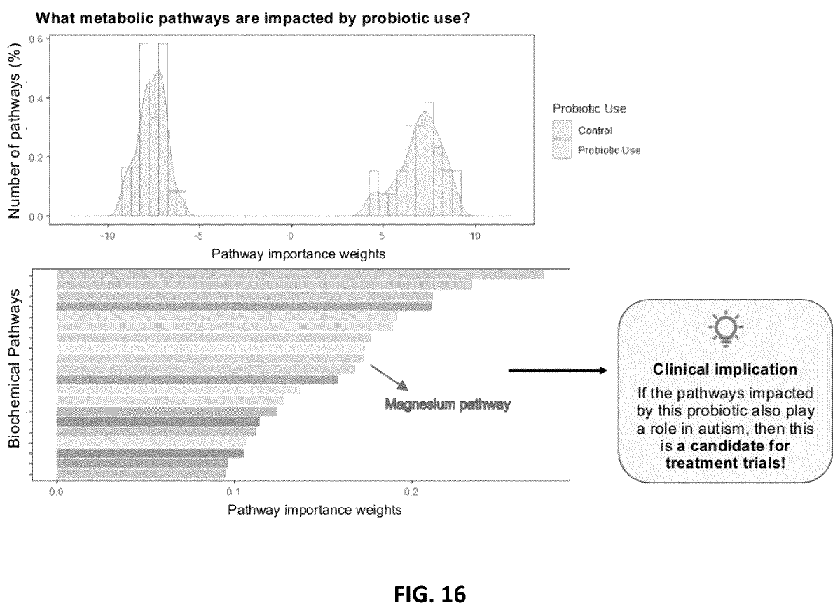

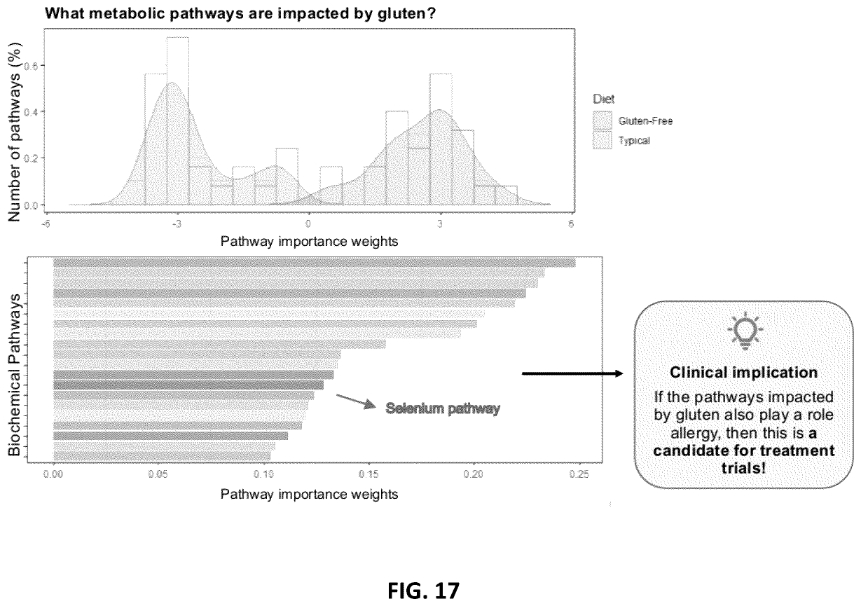

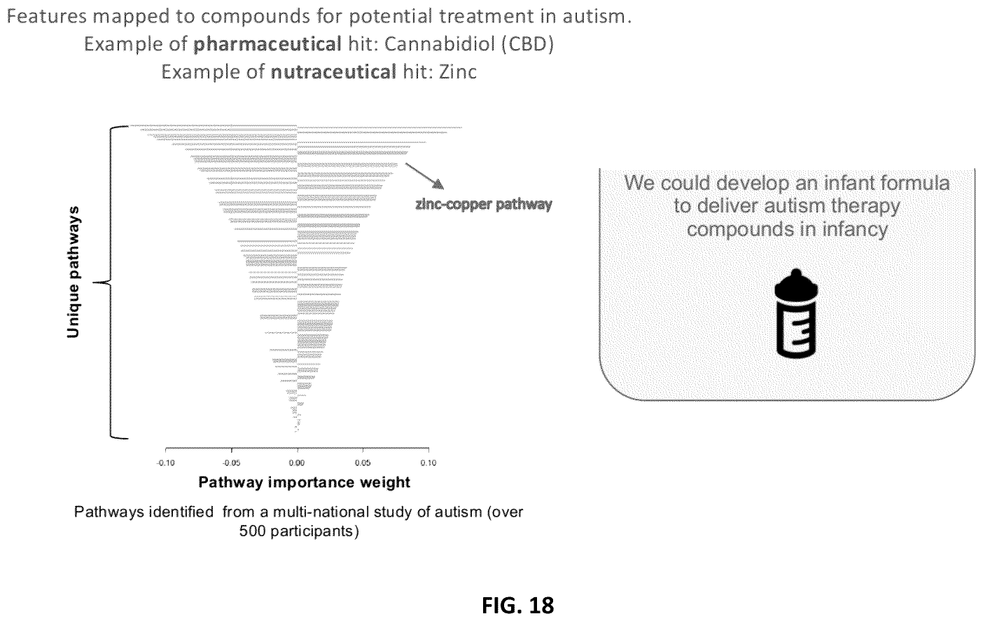

In some embodiments, the methods of optimal pharmaceutical and/or nutraceutical recommendation for subjects is used to analyze the influence and/or importance of exposome biochemical signature pathways impacted by intervention (e.g., pharmaceutical and/or nutraceuticals), as seen in FIGS. 16-18. In some instances, the features of the exposome biochemical signature pathways of an intervention and a control group may be compared. In some embodiments the intervention comprises probiotic use (FIG. 16), a gluten free diet (FIG. 17), cannabidiol, zinc (FIG. 18), or any combination thereof. In some instances, the intervention may be delivered as infant formula (FIG. 18). The outcome of the features of the exposome biochemical signature pathway analysis may comprise a recommended intervention with exposome biochemical signature pathways complementary or otherwise impacted in a similar manner by space travel.

### Determining Health Outcomes

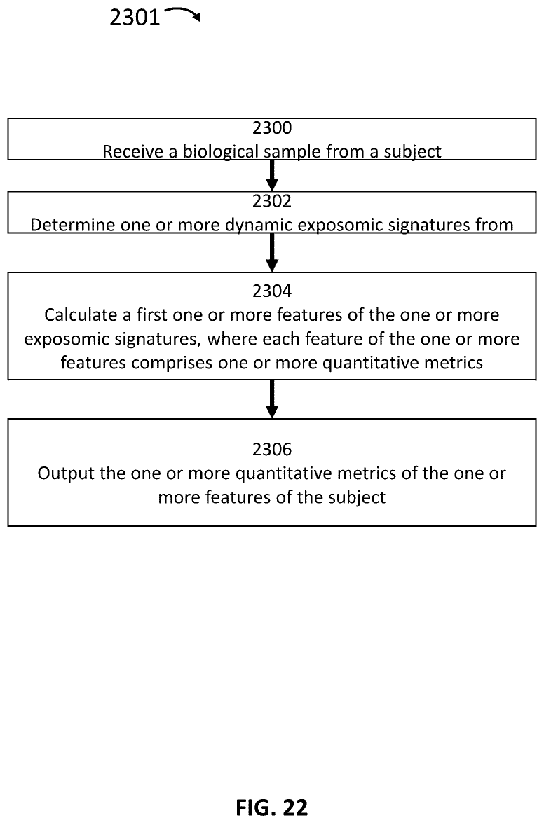

In some embodiments, the disclosure describes a method for outputting one or more quantitative metrics of a subject's one or more dynamic exposomic features 2301, as seen in FIG. 22. The method may comprise the following operations: (a) receiving a biological sample from a subject 2300; (b) determining one or more dynamic exposomic signatures from the biological sample of the subject 2302; (c) calculating a first one or more features of the one or more dynamic exposomic signatures, where each feature of the one or more features comprises one or more quantitative metrics 2304; and (d) outputting the one or more quantitative metrics of the one or more features of the subject 2306.

In some embodiments, the method may further comprise outputting a health outcome of the subject based at least in part on an association of normalized scores of the subject's one or more features to normalized scores of a second set of subjects' one or more features' one or more quantitative metrics. In some instances, the second set of subjects' one or more features' one or more quantitative metrics may be stored in a database, where the database is a local server or a cloud-based server. In some instances, the health outcome may comprise clinical subtype, non-clinical subgrouping related to physiology, anthropometric indicators, behavior indicators, socioeconomic indicators, body mass index, intelligence quotient, socio-economic status, or any combination thereof. In some embodiments, the one or more features may comprise a measurement of temporal dynamics of the one or more dynamic exposomic signatures.

In some instances, the measurement of the temporal dynamics may comprise: linear slope, non-linear parameters describing curvature of the one or more dynamic exposomic signatures, abrupt changes in intensity of the one or more dynamic exposomic signatures, changes in baseline intensity of the one or more dynamic exposomic signatures, changes of the frequency-domain representation of the one or more dynamic exposomic signatures, changes of the power-spectral domain representation of the one or more dynamic exposomic signatures, recurrence quantification analysis parameters, cross-recurrence quantification analysis parameters, joint recurrence quantification analysis parameters, multidimensional recurrence quantification analysis parameters, estimation of the Lyapunov spectra, maximum Lyapunov exponent, or any combination thereof.

In some instances, the one or more features of the one or more dynamic signatures may comprise phenotypic features, where the phenotypic features comprise: electrocardiogram (ECG), electroencephalography, magnetic resonance imaging (MRI), functional magnetic resonance imaging (fMRI), positron emission tomography (PET), genomic, epigenomic, transcriptomic, proteomic, metabolomic, or any combination thereof data. In some embodiments, the one or more features are derived from one or more attractors. In some instances, the one or more dynamic exposomic signatures may be measured by mass spectrometry, laser ablation-inductively coupled plasma mass spectrometry, laser induced breakdown spectroscopy, Raman spectroscopy, immunohistochemistry fluorescence, or any combination thereof. In some instances, the biological sample may comprise hair, teeth, toenails, fingernails, physiologic parameters, or any combination thereof. In some instances, the phenotypic features may comprise molecular phenotypes. In some instances, the molecular phenotypes may be determined by supervised or unsupervised analysis, where supervised or unsupervised analysis may comprise clustering, dimensionality-reduction, factor analysis, stacked autoencoding, or any combination thereof.

In some embodiments, the recurrence quantification analysis parameters comprise recurrence rates, determinism, mean diagonal length, maximum diagonal length, divergence, Shannon entropy in diagonal length, trend in recurrences, laminarity, trapping time, maximum vertical line length, Shannon entropy in vertical line lengths, mean recurrence time, Shannon entropy in recurrence times, and number of the most probable recurrences. In some embodiments, the method further comprises analyzing the one or more attractors by potential energy analysis thereby producing a potential energy data space. In some instances, the subject's one or more dynamic exposomic signatures may comprise retrospective, prospective, or any combination thereof dynamic exposomic data. In some instances, the method may further comprise analyzing a dynamic relationship between the one or more attractors' signal recurrence rates, determinism, mean diagonal length, maximum diagonal length, divergence, Shannon entropy in diagonal length, trend in recurrences, laminarity, trapping time, maximum vertical line length, Shannon entropy in vertical line lengths, mean recurrence time, Shannon entropy in recurrence times, number of the most probable recurrences, or any combination thereof. In some embodiments, the dynamic relationship is determined by cross-convergent mapping (CCM). The method of claim 1, further comprising reducing the one or more dynamic exposomic signatures to a reduced one or more exposomic dynamic signatures. In some instances, the method may further comprise constructing a network based on similarity of the one or more attractors' temporal exposomic data signal recurrence rates, determinism, mean diagonal length, maximum diagonal length, divergence, Shannon entropy in diagonal length, trend in recurrences, laminarity, trapping time, maximum vertical line length, Shannon entropy in vertical line lengths, mean recurrence time, Shannon entropy in recurrence times, number of the most probable recurrences, or any combination thereof. In some instances, the method may further comprise analyzing one or more features of the network to determine network connectivity, efficiency, feature importance, pathway importance, related graph-theory based metrics, or any combination thereof.

### Predicting Phenotypic Data

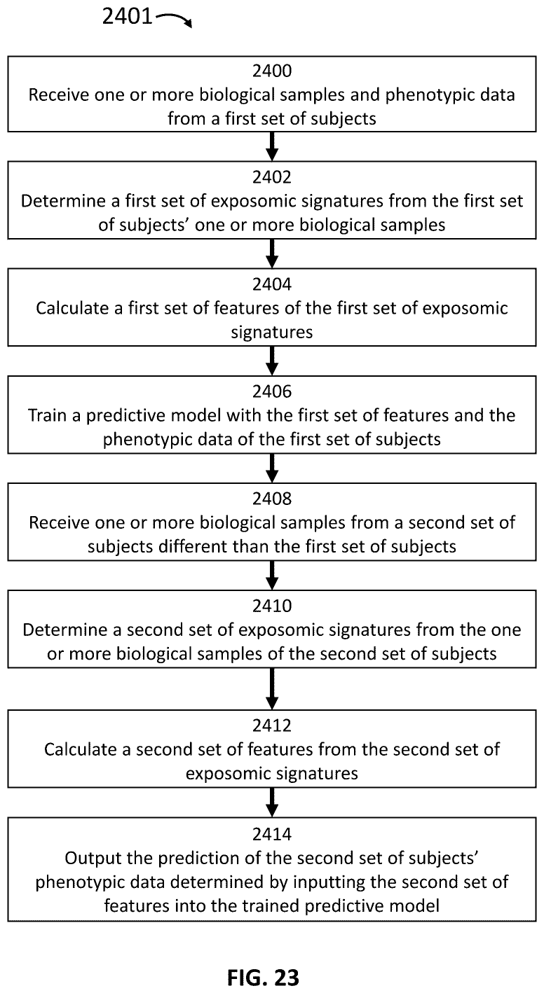

In some embodiments, the disclosure describes a method for outputting a prediction of one or more subjects' phenotypic data 2401, as seen in FIG. 23. The method may comprise the following operations: (a) receiving one or more biological samples and phenotypic data from a first set of subjects 2400; (b) determining a first set of exposomic signatures from the first set of subjects' one or more biological samples 2402; (c) calculating a first set of features of the first set of exposomic signatures 2404; (d) training a predictive model with the first set of features and the phenotypic data of the first set of subjects 2406; (e) receiving one or more biological samples from a second set of subjects different than the first set of subjects 2408; (f) determining a second set of exposomic signatures from the one or more biological samples of the second set of subjects 2410; (g) calculating a second set of features from the second set of exposomic signatures 2412; and (h) outputting the prediction of the second set of subjects' phenotypic data determined by inputting the second set of features into the trained predictive model 2414.

In some embodiments, the first and second set of features comprise one or more quantitative metrics. In some instances, the one or more quantitative metrics may comprise a measurement of temporal dynamics of the first and second set of exposomic signatures. In some instances, the measurement of the temporal dynamics may comprise: linear slope, non-linear parameters describing curvature of the first and second set of exposomic signatures, abrupt changes in intensity of the first and second set of exposomic signatures, changes in baseline intensity of the first and second set of exposomic signatures, changes of the frequency-domain representation of the first and second set of exposomic signatures, changes of the power-spectral domain representation of the first and second set of exposomic signatures, recurrence quantification analysis parameters, cross-recurrence quantification analysis parameters, joint recurrence quantification analysis parameters, multidimensional recurrence quantification analysis parameters, estimation of the Lyapunov spectra or maximum Lyapunov exponent, or any combination thereof.

In some instances, the first and second set of features may be represented as or derived from one or more attractors. In some embodiments, the one or more attractors is a limit cycle attractor, bistable attractor, or any combination thereof. In some embodiments, the first and second set of exposomic signatures are measured by mass spectrometry, laser ablation-inductively coupled plasma mass spectrometry, Raman spectroscopy, immunohistochemistry fluorescence, or any combination thereof. In some instances, the one or more biological sample of the first and second subjects may comprise hair, teeth, toenails, fingernails, physiologic parameters, or any combination thereof.

In some embodiments, the phenotypic features comprise molecular phenotypes. In some instances, the molecular phenotypes may be determined by unsupervised analysis, where unsupervised analysis comprises clustering, dimensionality-reduction, factor analysis, stacked autoencoding, or any combination thereof. In some instances, the recurrence quantification analysis parameters may comprise recurrence rates, determinism, mean diagonal length, maximum diagonal length, divergence, Shannon entropy in diagonal length, trend in recurrences, laminarity, trapping time, maximum vertical line length, Shannon entropy in vertical line lengths, mean recurrence time, Shannon entropy in recurrence times, and number of the most probable recurrences, or any combination thereof.

In some instances, the method may further comprise analyzing the one or more attractor by potential energy analysis thereby producing a potential energy data space. In some embodiments, the first and second set of exposomic signatures comprises retrospective, prospective, or any combination thereof exposomic data.

In some embodiments, the method further analyzes a dynamic relationship between the one or more attractors' recurrence rates, determinism, mean diagonal length, maximum diagonal length, divergence, Shannon entropy in diagonal length, trend in recurrences, laminarity, trapping time, maximum vertical line length, Shannon entropy in vertical line lengths, mean recurrence time, Shannon entropy in recurrence times, a number of the most probable recurrences, or any combination thereof. In some embodiments, the dynamic relationship is determined by cross-convergent mapping (CCM).

In some instances, the method may further comprise constructing a network based on similarity of the one or more attractors' temporal exposomic data signal recurrence rates, determinism, mean diagonal length, maximum diagonal length, divergence, Shannon entropy in diagonal length, trend in recurrences, laminarity, trapping time, maximum vertical line length, Shannon entropy in vertical line lengths, mean recurrence time, Shannon entropy in recurrence times, and number of the most probable recurrences, or any combination thereof. In some embodiments, the method further comprises analyzing one or more features of the network to determine network connectivity, efficiency, feature importance, pathway importance, related graph-theory based metrics feature importance, pathway importance, or any combination thereof.

Although the above steps show each of the methods or sets of operations, a person of ordinary skill in the art will recognize many variations based on the teaching described herein. The steps may be completed in a different order. Steps may be added or omitted. Some of the steps may comprise sub-steps. Many of the steps may be repeated as often as beneficial.

One or more of the steps of each of the methods or sets of operations may be performed with circuitry as described herein, for example, one or more of the processor or logic circuitry such as programmable array logic for a field programmable gate array. The circuitry may be programmed to provide one or more of the steps of each of the methods or sets of operations described elsewhere herein and the program may comprise program instructions stored on a computer readable memory or programmed steps of the logic circuitry such as the programmable array logic or the field programmable gate array, for example.

### Applications to Space Health Exposomics

In some aspects, the present disclosure describes a method for outputting one or more subjects' response to participating in real or simulated space travel. Various exposures may affect a subject during real or simulated space travel. In some embodiments, real or simulated space travel may expose a subject to increased levels of radiation, increased in levels of high-energy radiation (e.g., gamma, micro, X, and UV radiation), large G-forces, prolonged low G-forces, altered atmospheric pressure, altered atmosphere composition, or any combination thereof. In some cases, real or simulated space travel may lead to effects such as motion sickness, changes in bone density, bone deterioration, muscle deterioration, fluid redistribution, changes in cranial pressure, disruption of senses, psychological disorders, or any combination thereof. The various exposures and/or various effects of real or simulated space travel may create exposomic signatures in the subject's body or portions thereof. In some embodiments, the exposomic signatures associated with space travel may be detectable or be detected by a method of the present disclosure. In some embodiments, the method comprises receiving one or more biological samples of one or more subjects participating in space travel. In some embodiments, the method comprises determining one or more exposomic signatures of the one or more biological samples. In some embodiments, the method comprises calculating exposomic features of the one or more biological samples' one or more exposomic signatures.

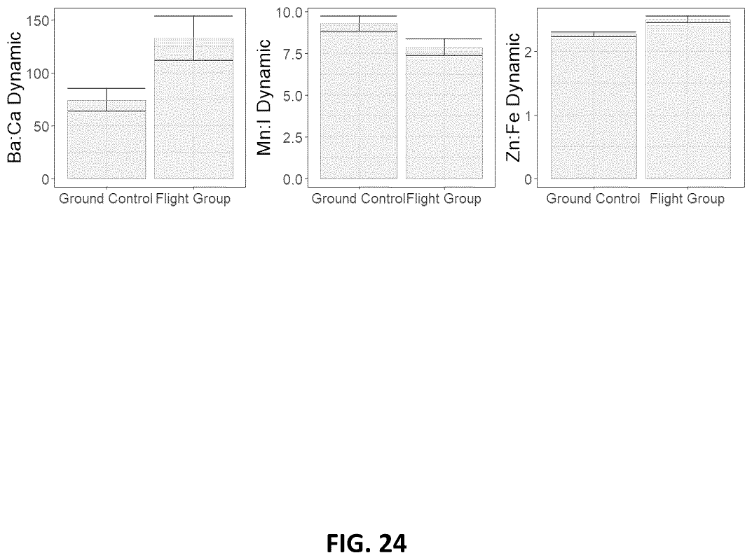

FIG. 24 shows quantitative metrics derived from cross-recurrent quantification analysis of signal dyads from control animals housed in a ground facility and mice that traveled to the international space Station, as seen in some embodiments. Several significant differences in elemental dynamics are shown between mice that experienced space travel and mice that did not, including perturbation of barium: calcium dynamics (P=0.02), manganese: iodine dynamics (P=0.04), and zinc: iron (P=0.002) dynamics. These patterns of dysregulation may constitute a detectable signature of space travel. In some cases, the detectable signatures may serve as a biomarker of space flight or as a target of ameliorative interventions.

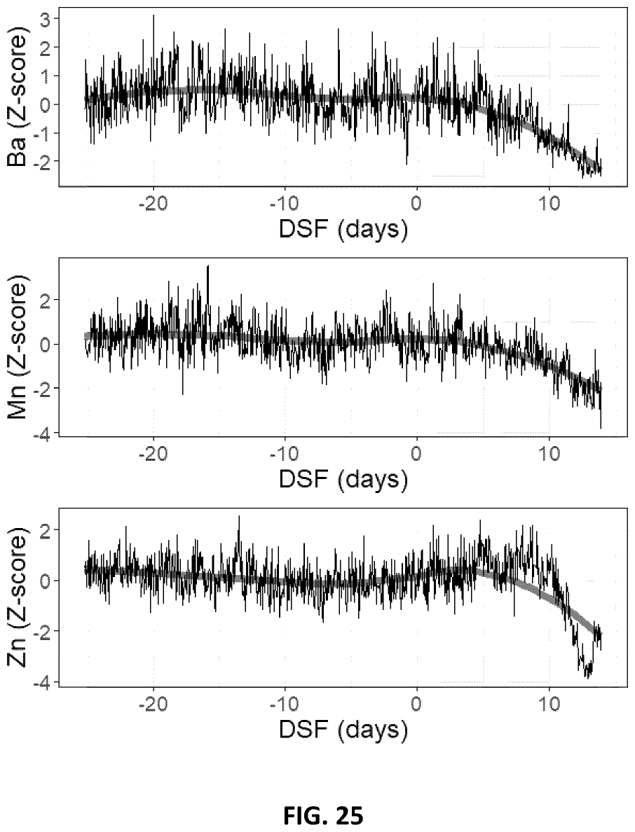

FIG. 25 shows data derived from analysis of barium, manganese, and zinc signals of human hair collected after space travel, as seen in some embodiments. A time-varying (expressed in days) magnitude of these signals relative to the timing of space flight, with the day of space flight identified as day 0 can be observed in the signals. Notable changes in barium, manganese, and zinc dynamics were identified following space flight. This pattern of change provides a signature of spaceflight which may be used as a biomarker of stress or a target in ameliorative efforts. Though specific embodiments of exposomic signatures were considered in this analysis, various other exposomic signatures may also provide signatures for space travel.

In some embodiments, the method comprises outputting the one or more subjects' response to participating in space travel by providing the exposomic features of the one or more biological samples as an input into a trained predictive model. In some embodiments, the trained predictive model is configured to determine a likelihood that the exposomic features are associated with metabolic signatures of one or more subjects participating in space travel. In some embodiments, metabolic signatures may be indicative of a current symptom, an underlying cause of a current symptom, a prediction of a symptom, a prediction of development of an ailment, or any combination thereof.

In some embodiments, the trained predictive model is configured to determine if subjects' metabolism is at baseline conditions not associated with space travel. In some embodiments, the base conditions may be based at least partially on measurements conducted on the subjects when they were on Earth. In some embodiments, the base conditions may be based at least partially on measurements conducted on the subjects after they are in space for a predetermined duration of time (e.g., 1 week, 1 month, etc.).

In some embodiments, each feature of the exposomic features comprises one or more quantitative metrics. Various examples to quantitative metrics are disclosed elsewhere in the present application.

In some embodiments, the one or more biological samples of the subjects participating in the space travel is collected prior to the space travel, during the space travel, after the space travel, or any combination thereof periods of the space travel. In some embodiments, the one or more biological samples, when collected prior to the space travel, may be analyzed to determine a suitability of a given subject for space travel. In some embodiments, the one or more biological samples, when collected during the space travel, may be analyzed to determine therapeutic intervention for a given subject. In some embodiments, the one or more biological samples, when collected after the space travel, may be analyzed to determine recovery intervention for a given subject.

In some embodiments, the exposomic features comprise a measurement of temporal dynamics of the one or more exposomic signatures. In some embodiments, the measurement of the temporal dynamics comprises: determination of a linear slope, determination of a plurality of non-linear parameters describing curvature of the one or more exposomic signatures, determination of an abrupt change in intensity of the one or more exposomic signatures, determination of one or more changes in a baseline intensity of the one or more exposomic signatures, determination of a change of a frequency-domain representation of the one or more exposomic signatures, determination of a change of the power-spectral domain representation of the one or more exposomic signatures, determination of one or more recurrence quantification analysis parameters, determination of one or more cross-recurrence quantification analysis parameters, determination of one or more joint recurrence quantification analysis parameters, determination of one or more multidimensional recurrence quantification analysis parameters, estimation of a Lyapunov spectra, determination of a maximum Lyapunov exponent, or any combination thereof.

In some embodiments, the measurement of the temporal dynamics comprises determination of one or more recurrence quantification analysis parameters, wherein the one or more recurrence quantification analysis parameters comprises a recurrence rate, a determinism, a mean diagonal length, a maximum diagonal length, a divergence, a Shannon entropy in diagonal length, a trend in recurrence, a laminarity, a trapping time, a maximum vertical line length, a Shannon entropy in vertical line length, a mean recurrence time, a Shannon entropy in recurrence time, or a number of a most probable recurrence.

In some embodiments, the one or more exposomic signatures are measured by mass spectrometry, laser ablation-inductively coupled plasma-mass spectrometry, laser induced breakdown spectroscopy, Raman spectroscopy, immunohistochemistry fluorescence, or any combination thereof.

In some embodiments, the exposomic features are derived from one or more attractors. In some embodiments, the method further comprises analyzing a dynamic relationship between the one or more attractors' signal recurrence rates, determinism, mean diagonal length, maximum diagonal length, divergence, Shannon entropy in diagonal length, trend in recurrences, laminarity, trapping time, maximum vertical line length, Shannon entropy in vertical line lengths, mean recurrence time, Shannon entropy in recurrence times, number of the most probable recurrences, or any combination thereof.

In some embodiments, the trained predictive model comprises a classification algorithm. In some embodiments, the classification algorithm is configured to identify samples associated with participating in space travel. In some embodiments, the classification algorithm is trained with the one or more subjects' one or more biological samples exposomic features and corresponding responses. In some embodiments, the one or more subjects participated in space travel. In some embodiments, the classification algorithm is configured to determine if the one or more subjects have a metabolic signature associated with space travel.

In some embodiments, the response comprises a state of the subject's metabolism. In some embodiments, the subject's metabolism comprises a healthy state or a state consistent with a subject undergoing space-travel.

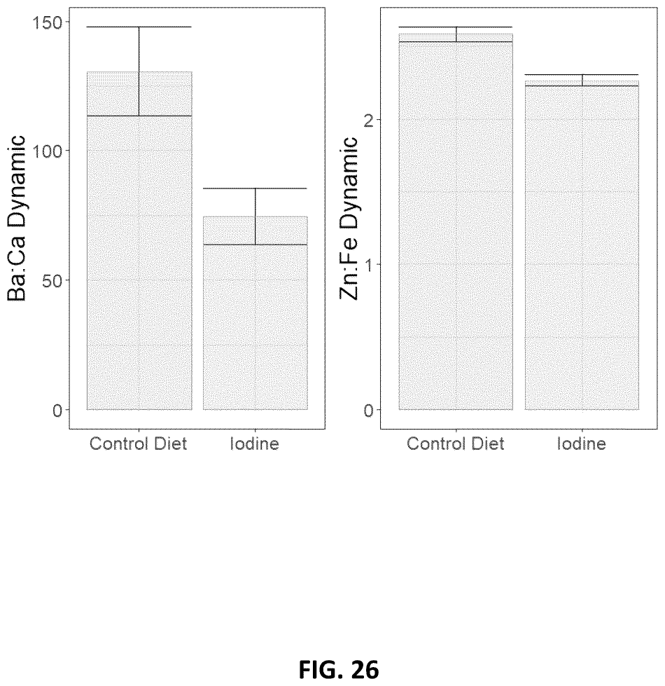

FIG. 26 shows mean Ba: CA dynamics, Zn: Fe dynamics in mice that received a control diet or received iodine supplementation to their diet, as seen in some embodiments. Iodine supplementation significantly reduces levels of barium: calcium (P=0.01) and zinc: iron (P=0.0001) dynamics. In some cases, space travel reduces levels of these signatures, and the results show how the effects of ameliorative treatments may be linked to the action of ameliorative effects associated with a molecular mechanism and/or effector.

FIG. 37 shows mean Ba: CA dynamics, Zn: Fe dynamics in mice that received a diet of food bars or received a standard lab chow diet, as seen in some embodiments. It can be seen that dietary manipulation can yield significant changes in levels of barium: calcium (P=0.004) and zinc: iron (P=0.02) dynamics. The results show that the effects of ameliorative treatments, for example nutritional interventions, and links between the action of ameliorative effects with a molecular mechanism and/or effector.

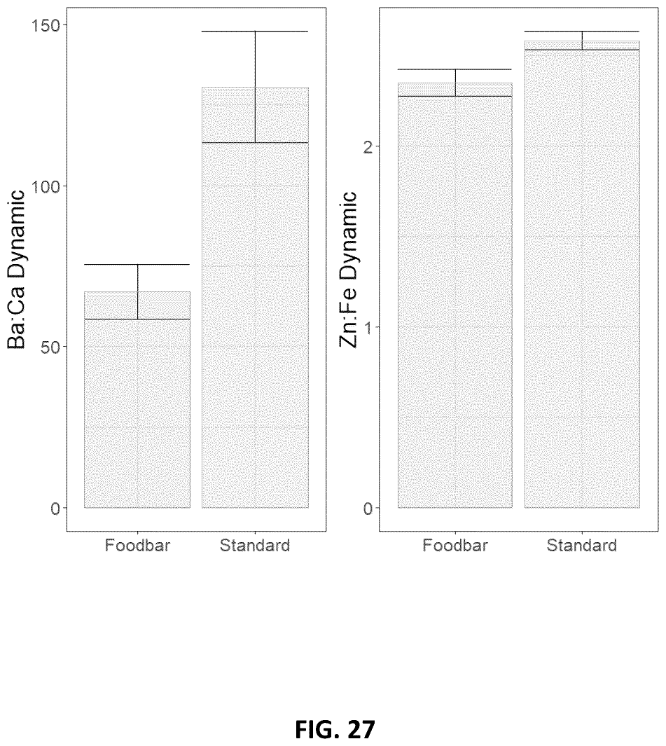

The studies illustrated by FIGS. 26 and 27 show that statistically significant exposomic signatures may be induced in organisms undergoing space travel. In various embodiments disclosed herein, the exposomic signatures may be serve as basis for training machine learning algorithms to detect signs of associated with space travel, tolerance to space travel, likelihood of a therapeutic intervention to treat a condition associated with space travel, for example. In various embodiments disclosed herein, the exposomic signatures may serve as basis for using machine learning algorithms to predict subjects' tolerance to space travel, or likelihood of a therapeutic intervention to treat a condition associated with space travel, for example.

In some aspects, the present disclosure describes a method for training a predictive model to determine a clinical response of subjects to an intervention when the subject is participating in space travel. In some embodiments, the method comprises receiving one or more biological samples and clinical responses of a first set of subjects. In some embodiments, the first set of subjects are exposed to space travel and are provided an intervention. In some embodiments, the method comprises receiving one or more biological samples and clinical responses of a second set of subjects. In some embodiments, the second set of subjects are exposed to space travel and do not receive the intervention. In some embodiments, the method comprises determining one or more exposomic signatures of the one or more biological samples of the first and second set of subjects. In some embodiments, the method comprises calculating a first and second set of exposomic features of the first and second set of subjects' one or more exposomic signatures. In some embodiments, each feature of the first and second set of exposomic features comprises one or more quantitative metrics. In some embodiments, the method comprises outputting a trained predictive model configured to determine a clinical response of a subject to an intervention. In some embodiments, the subject is participating in space travel. In some embodiments, the trained predictive model is trained on the first and second set of subjects' one or more exposomic features' one or more quantitative metrics and the corresponding clinical responses. In some embodiments, the intervention prevents negative side effects of space travel. In some embodiments, the intervention is provided to the first set of subjects prior to, during, after, or any combination thereof time point of space travel the subjects have been exposed to. In some embodiments, the intervention comprises a diet, pharmaceutical therapeutic, or any combination thereof. In some embodiments, the diet comprises iodine supplementation.

In some embodiments, the method further comprises determining an influence of space travel on the first set of subjects exposed to the intervention at least in part by comparing the first and second set of exposomic features' one or more quantitative metrics.

In some embodiments, the first or second set of exposomic features comprise a measurement of temporal dynamics of the one or more exposomic signatures. In some embodiments, the measurement of the temporal dynamics comprises: determination of a linear slope, determination of a plurality of non-linear parameters describing curvature of the one or more exposomic signatures, determination of an abrupt change in intensity of the one or more exposomic signatures, determination of one or more changes in a baseline intensity of the one or more exposomic signatures, determination of a change of a frequency-domain representation of the one or more exposomic signatures, determination of a change of the power-spectral domain representation of the one or more exposomic signatures, determination of one or more recurrence quantification analysis parameters, determination of one or more cross-recurrence quantification analysis parameters, determination of one or more joint recurrence quantification analysis parameters, determination of one or more multidimensional recurrence quantification analysis parameters, estimation of a Lyapunov spectra, determination of a maximum Lyapunov exponent, or any combination thereof. In some embodiments, the measurement of the temporal dynamics comprises determination of one or more the recurrence quantification analysis parameters, wherein the one or more the recurrence quantification analysis parameters comprises a recurrence rate, a determinism, a mean diagonal length, a maximum diagonal length, a divergence, a Shannon entropy in diagonal length, a trend in recurrence, a laminarity, a trapping time, a maximum vertical line length, a Shannon entropy in vertical line length, a mean recurrence time, a Shannon entropy in recurrence time, or a number of a most probable recurrence. Various statistical analysis of exposomic signature may be used.

In some embodiments, the one or more exposomic signatures are measured by mass spectrometry, laser ablation-inductively coupled plasma-mass spectrometry, laser induced breakdown spectroscopy, Raman spectroscopy, immunohistochemistry fluorescence, or any combination thereof. In some embodiments, the one or more exposomic signatures may be measured from genetic sequencing, proteomics, or both.

In some embodiments, the one or more biological samples comprise hair, teeth, toenails, fingernails, physiologic parameters, or any combination thereof.

In some embodiments, the first or second set of exposomic features are derived from one or more attractors. In some embodiments, the method further comprises analyzing a dynamic relationship between the one or more attractors' signal recurrence rates, determinism, mean diagonal length, maximum diagonal length, divergence, Shannon entropy in diagonal length, trend in recurrences, laminarity, trapping time, maximum vertical line length, Shannon entropy in vertical line lengths, mean recurrence time, Shannon entropy in recurrence times, number of the most probable recurrences, or any combination thereof.

In some embodiments, the first or second set of subjects are human, non-human mammals, or any combination thereof.

In some aspects, the present disclosure describes a method for generating a predictive model configured to output one or more subjects' health state in response to a gravitational force in addition to the earth's gravitational force. In some embodiments, the method comprises receiving a first one or more subjects' one or more biological samples and corresponding health states. In some embodiments, the first one or more subjects' have received an exposure to a gravitational force in addition to the earth's gravitational force. In some embodiments, the method comprises determining one or more exposomic signatures of the first one or more subjects' one or more biological samples. In some embodiments, the method comprises calculating exposomic features of the one or more biological samples' one or more exposomic signatures. In some embodiments, the method comprises generating a trained predictive mode configured to output a second one or more subjects' health state in response to a gravitational force in addition to the earth's gravitational force. In some embodiments, the trained predictive model is trained with the first one or more subjects' exposomic features of the one or more exposomic signatures and corresponding health states.

In some embodiments, the gravitational force comprises at least 1 G, at least 2 G, at least 3G, or at least 4G. In some embodiments, the trained predictive model is configured to determine a likelihood that the exposomic features are associated with metabolic signatures of the first or second one or more subject exposed to a gravitational force in addition to the earth's gravitational force. In some embodiments, a probabilistic threshold determined by the trained predictive model indicates that the first or second one or more subjects' metabolism is at baseline conditions not associated with an exposure to a gravitational force in addition to the earth's gravitational force.

In some embodiments, the exposomic features comprise a measurement of temporal dynamics of the one or more exposomic signatures. In some embodiments, the measurement of the temporal dynamics comprises: determination of a linear slope, determination of a plurality of non-linear parameters describing curvature of the one or more exposomic signatures, determination of an abrupt change in intensity of the one or more exposomic signatures, determination of one or more changes in a baseline intensity of the one or more exposomic signatures, determination of a change of a frequency-domain representation of the one or more exposomic signatures, determination of a change of the power-spectral domain representation of the one or more exposomic signatures, determination of one or more recurrence quantification analysis parameters, determination of one or more cross-recurrence quantification analysis parameters, determination of one or more joint recurrence quantification analysis parameters, determination of one or more multidimensional recurrence quantification analysis parameters, estimation of a Lyapunov spectra, determination of a maximum Lyapunov exponent, or any combination thereof. In some embodiments, the measurement of the temporal dynamics comprises determination of one or more recurrence quantification analysis parameters, wherein the one or more recurrence quantification analysis parameters comprises a recurrence rate, a determinism, a mean diagonal length, a maximum diagonal length, a divergence, a Shannon entropy in diagonal length, a trend in recurrence, a laminarity, a trapping time, a maximum vertical line length, a Shannon entropy in vertical line length, a mean recurrence time, a Shannon entropy in recurrence time, or a number of a most probable recurrence.

In some embodiments, the one or more exposomic signatures are measured by mass spectrometry, laser ablation-inductively coupled plasma-mass spectrometry, laser induced breakdown spectroscopy, Raman spectroscopy, immunohistochemistry fluorescence, or any combination thereof.

In some embodiments, the exposomic features are derived from one or more attractors. In some embodiments, the method further comprises analyzing a dynamic relationship between the one or more attractors' signal recurrence rates, determinism, mean diagonal length, maximum diagonal length, divergence, Shannon entropy in diagonal length, trend in recurrences, laminarity, trapping time, maximum vertical line length, Shannon entropy in vertical line lengths, mean recurrence time, Shannon entropy in recurrence times, number of the most probable recurrences, or any combination thereof.

In some embodiments, the first and second one or more subjects comprise human subjects, non-human mammal subjects, vertebrate subjects, or any combination thereof.

In some embodiments, the trained predictive model comprises a classification algorithm. In some embodiments, the classification algorithm is configured to identify the one or more exposomic signatures of the first or second one or more subject associated with exposure to the gravitational force in addition to the earth's gravitational force. In some embodiments, the classification algorithm is trained with the first one or more subjects' one or more biological samples' exposomic features and corresponding health states. In some embodiments, the first one or more one or more subjects were exposed to the gravitational force in addition to the earth's gravitational force. In some embodiments, the classification algorithm is configured to determine if the first or second one or more subjects' have metabolic signatures associated with exposure to the gravitational force in addition to the earth's gravitational force.

In some embodiments, the health state comprises a state of the first or second one or more subjects' metabolism. In some embodiments, the first or second one or more subjects' metabolism comprises a healthy state or a state consistent with a subject exposed to the gravitational force in addition to the earth's gravitational force.

In some aspects, the present disclosure describes a method for using a predictive model to output a health outcome of one or more subjects prior to exposure of a gravitational force. In some embodiments, the method comprises receiving one or more subjects' one or more biological samples. In some embodiments, the method comprises determining one or more exposomic signatures of the one or more subjects' one or more biological samples. In some embodiments, the method comprises calculating exposomic features of the one or more biological samples' one or more exposomic signatures. In some embodiments, the method comprises outputting a health outcome of the one or more subjects prior to exposure of a gravitational force in addition to the gravitational force of the earth by providing the one or more subjects' exposomic features as an input to a trained predictive model.

In some embodiments, the gravitational force comprises at least 1 G, at least 2 G, at least 3G, or at least 4G. In some embodiments, the gravitational force comprises at least 5, 6, 7, 8, 9, or 10 G. In some embodiments, the gravitational force comprises at most 5, 6, 7, 8, 9, or 10 G.

In some embodiments, the trained predictive model is configured to determine a likelihood that the exposomic features are associated with metabolic signatures of the one or more subjects exposed to the gravitational force.

In some embodiments, a probabilistic threshold determined by the trained predictive model indicates that one or more subjects' metabolism is at baseline conditions not associated with the exposure to the gravitational force in addition to the earth's gravitational force.

In some embodiments, each feature of the exposomic features comprises one or more quantitative metrics.

In some embodiments, the one or more biological samples comprise hair, teeth, toenails, fingernails, physiologic parameters, or any combination thereof.

In some embodiments, the exposomic features comprise a measurement of temporal dynamics of the one or more exposomic signatures.

In some embodiments, the measurement of the temporal dynamics comprises: determination of a linear slope, determination of a plurality of non-linear parameters describing curvature of the one or more exposomic signatures, determination of an abrupt change in intensity of the one or more exposomic signatures, determination of one or more changes in a baseline intensity of the one or more exposomic signatures, determination of a change of a frequency-domain representation of the one or more exposomic signatures, determination of a change of the power-spectral domain representation of the one or more exposomic signatures, determination of one or more recurrence quantification analysis parameters, determination of one or more cross-recurrence quantification analysis parameters, determination of one or more joint recurrence quantification analysis parameters, determination of one or more multidimensional recurrence quantification analysis parameters, estimation of a Lyapunov spectra, determination of a maximum Lyapunov exponent, or any combination thereof.

In some embodiments, the measurement of the temporal dynamics comprises determination of one or more recurrence quantification analysis parameters, wherein the one or more recurrence quantification analysis parameters comprises a recurrence rate, a determinism, a mean diagonal length, a maximum diagonal length, a divergence, a Shannon entropy in diagonal length, a trend in recurrence, a laminarity, a trapping time, a maximum vertical line length, a Shannon entropy in vertical line length, a mean recurrence time, a Shannon entropy in recurrence time, or a number of a most probable recurrence.

In some embodiments, the one or more exposomic signatures are measured by mass spectrometry, laser ablation-inductively coupled plasma-mass spectrometry, laser induced breakdown spectroscopy, Raman spectroscopy, immunohistochemistry fluorescence, or any combination thereof.

In some embodiments, the exposomic features are derived from one or more attractors.

In some embodiments, the method further comprises analyzing a dynamic relationship between the one or more attractors' signal recurrence rates, determinism, mean diagonal length, maximum diagonal length, divergence, Shannon entropy in diagonal length, trend in recurrences, laminarity, trapping time, maximum vertical line length, Shannon entropy in vertical line lengths, mean recurrence time, Shannon entropy in recurrence times, number of the most probable recurrences, or any combination thereof.

In some embodiments, the one or more subjects comprise a human, non-human mammal, vertebrate, or any combination thereof.

In some embodiments, the trained predictive model comprises a classification algorithm.

In some embodiments, the classification algorithm is configured to identify the one or more exposomic signatures of the subject associated with an exposure to the gravitational force in addition to the earth's gravitational force.

In some embodiments, the classification algorithm is trained with one or more subjects' one or more biological samples' exposomic features and corresponding responses. In some embodiments, the one or more subjects are exposed to a gravitational force in addition to the earth's gravitational force.

In some embodiments, the classification algorithm is configured to determine if the one or more subjects' have metabolic signatures associated with exposure to a gravitational force in addition to the earth's gravitational force.

In some aspects, the present disclosure describes a method for administering a treatment to a subject participating in space travel. In some embodiments, the method comprises receiving one or more biological samples of a subject prior to participating in space travel. In some embodiments, the method comprises determining one or more exposomic signatures of the one or more biological samples of the subject. In some embodiments, the method comprises calculating exposomic features of the subject's one or more exposomic signatures. In some embodiments, each feature of the exposomic features comprises one or more quantitative metrics. In some embodiments, the method comprises administering a treatment to the subject participating in space travel at a time point, wherein the treatment and the time point of administering the treatment is determined as an output of a trained predictive model when the trained predictive model is provided the exposomic features of the subject as an input. In some embodiments, the trained predictive model is configured to output an efficacy of the treatment.

In some embodiments, the trained predictive model is configured to output a measure of whether or not the treatment is efficacious.

In some embodiments, the time point comprises a time prior to, during, after, or any combination thereof time of the subject participating in the space travel.

In some embodiments, the treatment is provided to mitigate negative side-effects of participating in space travel. In some embodiments, the treatment may be provided prophylactically. In some embodiments, the treatment may be provided after space travel.

In some embodiments, the efficacy of the treatment is determined by the duration of time after the subject's participation in space travel after which the subject's exposomic features comprise baseline exposomic features of a subject that has not participated in space travel.

In some embodiments, the trained predictive model comprises a classification algorithm.

In some embodiments, the space travel comprises traveling beyond the Karman line at an altitude of at least 100 kilometers (km) above the Earth's surface.

In some embodiments, the one or more biological samples comprise hair, teeth, toenails, fingernails, physiologic parameters, or any combination thereof.

In some embodiments, the exposomic features comprise a measurement of temporal dynamics of the one or more exposomic signatures.

In some embodiments, the measurement of the temporal dynamics comprises: determination of a linear slope, determination of a plurality of non-linear parameters describing curvature of the one or more exposomic signatures, determination of an abrupt change in intensity of the one or more exposomic signatures, determination of one or more changes in a baseline intensity of the one or more exposomic signatures, determination of a change of a frequency-domain representation of the one or more exposomic signatures, determination of a change of the power-spectral domain representation of the one or more exposomic signatures, determination of one or more recurrence quantification analysis parameters, determination of one or more cross-recurrence quantification analysis parameters, determination of one or more joint recurrence quantification analysis parameters, determination of one or more multidimensional recurrence quantification analysis parameters, estimation of a Lyapunov spectra, determination of a maximum Lyapunov exponent, or any combination thereof.

In some embodiments, the measurement of the temporal dynamics comprises determination of one or more recurrence quantification analysis parameters, wherein the one or more recurrence quantification analysis parameters comprises a recurrence rate, a determinism, a mean diagonal length, a maximum diagonal length, a divergence, a Shannon entropy in diagonal length, a trend in recurrence, a laminarity, a trapping time, a maximum vertical line length, a Shannon entropy in vertical line length, a mean recurrence time, a Shannon entropy in recurrence time, or a number of a most probable recurrence.

In some embodiments, the one or more exposomic signatures are measured by mass spectrometry, laser ablation-inductively coupled plasma-mass spectrometry, laser induced breakdown spectroscopy, Raman spectroscopy, immunohistochemistry fluorescence, or any combination thereof.

In some embodiments, the exposomic features are derived from one or more attractors.

In some embodiments, the method further comprises analyzing a dynamic relationship between the one or more attractors' signal recurrence rates, determinism, mean diagonal length, maximum diagonal length, divergence, Shannon entropy in diagonal length, trend in recurrences, laminarity, trapping time, maximum vertical line length, Shannon entropy in vertical line lengths, mean recurrence time, Shannon entropy in recurrence times, number of the most probable recurrences, or any combination thereof.

In some embodiments, the subject is a human, non-human mammal, or any combination thereof.

In some embodiments, the trained predictive model comprises a classification algorithm.

In some embodiments, the classification algorithm is trained with one or more subjects' one or more biological samples' exposomic features, treatment administered, time point of administering the treatment, and corresponding clinical response, wherein the one or more subjects participated in space travel.

In some embodiments, the classification algorithm is configured to determine if one or more subjects have a dysregulated metabolism induced by the space travel. In some embodiments, a dysregulated metabolism may comprise a change in expression of a gene associated with metabolism, a mutation in a gene associated with metabolism, or both.

In some aspects, the present disclosure describes a method for evaluating one or more subjects' response to an intervention in the presence or lack thereof gravity. In some embodiments, the method comprises sampling each respective position in a plurality of positions along a reference line of a first set of one or more biological samples of a first set of subjects and a second set of one or more biological samples of a second set of subjects. In some embodiments, the method comprises obtaining a plurality of ion samples. In some embodiments, each ion sample in the plurality of ion samples corresponding to a different position in the plurality of positions. In some embodiments, each position in the plurality of positions representing a different period of growth of the first and second set of one or more biological samples. In some embodiments, the first set of subjects are subjected to space travel and the second set of subjects are not subjected to space travel. In some embodiments, the first and second set of subjects are administered an intervention.

In some embodiments, the method comprises analyzing each ion sample in the plurality of ion samples with a mass spectrometer. In some embodiments, the method comprises obtaining a first dataset that includes a plurality of traces. In some embodiments, each trace in the plurality of traces being a concentration of a corresponding elemental isotope, in a plurality of elemental isotopes, over time collectively determined from the plurality of ion samples.

In some embodiments, the method comprises deriving a second dataset from the plurality of traces that includes a set of features. In some embodiments, each respective feature in the set of features being determined by a variation of a single isotope or a combination of isotopes in the plurality of traces.

In some embodiments, the method comprises inputting the set of features into a trained classifier thereby obtaining a probability from the trained classifier of the first and second set of one or more subjects' which predicts the one or more subjects' response to the space travel.

In some embodiments, the probability indicates that a biological sample of the one or more biological samples has been dysregulated as a result of the space travel.

In some embodiments, an efficacy of the intervention is measured by the probability of dysregulation.

In some embodiments, the intervention comprises a diet, pharmaceutical therapeutic, or any combination thereof.

In some embodiments, the diet comprises iodine supplementation.

In some embodiments, the intervention ameliorates radiation exposure.

In some embodiments, the first or second set of subjects comprise a human, non-human mammal, or any combination thereof.

In some embodiments, the non-human mammal comprises mice.

In some embodiments, the first or second set of one or more biological samples comprise hair, teeth, toenails, fingernails, physiologic parameters, or any combination thereof.

In some embodiments, the method comprises treating the first or second set of one or more biological samples with a solvent or a surfactant prior to the sampling.

In some embodiments, the method comprises irradiating the first or second set of one or more biological samples with a low powered laser to remove debris from the first or second set of one or more biological samples prior to the sampling.

In some embodiments, the sampling includes irradiating, with a laser, the first or second set of one or more biological samples of the first or second set of subjects thereby extracting a plurality of particles from the first or second set of one or more biological samples. In some embodiments, the sampling includes ionizing the plurality of particles with an inductively coupled plasma mass spectrometer, thereby obtaining the plurality of ion samples.

In some embodiments, the first or second set of subjects' responses comprise adverse response, positive response, or neutral response.

In some embodiments, the trained classifier is configured to identify if a sample was collected from a subject that participated in space travel.

In some embodiments, the trained classifier is trained with the first set of subjects' one or more biological samples' set of features and corresponding responses.

In some embodiments, the trained classifier is configured to determine if a subject comprises a dysregulated metabolism induced by the space travel.

In some aspects, the present disclosure describes a method for outputting a subject's response to an intervention following space travel. In some embodiments, the method comprises exposing a subject's one or more biological samples to an optical signal comprising a plurality of Raman wavelengths. In some embodiments, the subject is subjected to space travel. In some embodiments, the method comprises acquiring a plurality of Raman spectra from the one or more biological samples. In some embodiments, the method comprises processing the plurality of Raman spectra to generate a plurality of Raman features. In some embodiments, the method comprises outputting a probability of a subject's response to an intervention when subjected to space travel as a result of providing a trained classifier an input of the plurality of Raman features.

In some embodiments, the probability indicates that the subject has been dysregulated as a result of the space travel.

In some embodiments, an efficacy of the intervention is measured by the probability of dysregulation.

In some embodiments, the one or more biological samples comprise hair, teeth, toenails, fingernails, physiologic parameters, or any combination thereof.

In some embodiments, the intervention comprises a diet, pharmaceutical therapeutic, or any combination thereof.

In some embodiments, the diet comprises iodine supplementation.

In some embodiments, the intervention ameliorates radiation exposure.

In some embodiments, the subject comprises a human, non-human mammal, or any combination thereof.

In some embodiments, the non-human mammal comprises mice.

In some embodiments, the plurality of Raman spectra comprises from about 200 to about 3700 wave numbers.

In some embodiments, the acquiring comprises using a Raman spectroscopy microscope.

In some embodiments, the plurality of Raman features comprise one or more traces derived from a Raman spatial map.

In some embodiments, the one or more traces are reduced using a dimensionality-reduction algorithm. In some embodiments, the dimensionality-reduction algorithm comprises principal component analyses, independent component analysis, or any combination thereof algorithm.

In some embodiments, a measurement of temporal dynamics is derived from the one or more traces, wherein the temporal dynamics comprise: determination of a linear slope, determination of a plurality of non-linear parameters describing curvature of the one or more exposomic signatures, determination of an abrupt change in intensity of the one or more exposomic signatures, determination of one or more changes in a baseline intensity of the one or more exposomic signatures, determination of a change of a frequency-domain representation of the one or more exposomic signatures, determination of a change of the power-spectral domain representation of the one or more exposomic signatures, determination of one or more recurrence quantification analysis parameters, determination of one or more cross-recurrence quantification analysis parameters, determination of one or more joint recurrence quantification analysis parameters, determination of one or more multidimensional recurrence quantification analysis parameters, estimation of a Lyapunov spectra, determination of a maximum Lyapunov exponent, or any combination thereof.

In some embodiments, the measurement of the temporal dynamics comprises determination of one or more the recurrence quantification analysis parameters, wherein the one or more the recurrence quantification analysis parameters comprises a recurrence rate, a determinism, a mean diagonal length, a maximum diagonal length, a divergence, a Shannon entropy in diagonal length, a trend in recurrence, a laminarity, a trapping time, a maximum vertical line length, a Shannon entropy in vertical line length, a mean recurrence time, a Shannon entropy in recurrence time, or a number of a most probable recurrence.

In some embodiments, the trained classifier comprises a gradient-boosted ensemble model.

In some embodiments, the trained classifier is configured to process one or more features selected from the group consisting of laminarity, entropy, trapping time (TT), mean diagonal length (MDL), recurrence time (RT), Vmax, determinism, Lmax, a maximum diagonal length, a divergence, a Shannon entropy in diagonal length, a trend in recurrence, a maximum vertical line length, a Shannon entropy in vertical line length, a mean recurrence time, a Shannon entropy in recurrence time, a number of a most probable recurrence, or any combination thereof.

In some embodiments, the trained classifier is configured to process two or more features selected from the group consisting of laminarity, entropy, trapping time (TT), mean diagonal length (MDL), recurrence time (RT), Vmax, determinism, Lmax, a maximum diagonal length, a divergence, a Shannon entropy in diagonal length, a trend in recurrence, a maximum vertical line length, a Shannon entropy in vertical line length, a mean recurrence time, a Shannon entropy in recurrence time, a number of a most probable recurrence, or any combination thereof.

In some embodiments, the trained classifier is configured to process 1, 2, 3, 4, 5, 6, 7, or 8 features selected from the group consisting of laminarity, entropy, trapping time (TT), mean diagonal length (MDL), recurrence time (RT), Vmax, determinism, Lmax, a maximum diagonal length, a divergence, a Shannon entropy in diagonal length, a trend in recurrence, a maximum vertical line length, a Shannon entropy in vertical line length, a mean recurrence time, a Shannon entropy in recurrence time, a number of a most probable recurrence, or any combination thereof.

In some embodiments, the trained classifier is trained to identify Raman signatures associated with space travel.

In some embodiments, the output probability of the trained classifier indicates that the subject is exhibiting temporal dynamics associated with space travel.

In some embodiments, the trained classifier is trained with a population of human or non-human animals' plurality of Raman features, wherein the population of human or non-human animals have experienced space travel.

In some embodiments, an efficacy of the intervention is measured by a change of the subject's temporal dynamics towards a baseline temporal dynamics of the subject prior to space travel.

In some aspects, the present disclosure descries a method for evaluating a subject's response to an intervention to reduce the biological effects of space travel. In some embodiments, the method comprises staining a first set of one or more biological samples of a first set of subject and a second set of one or more biological samples of a second set of subjects to produce a stained first and second set of one or more biological samples. In some embodiments, the first set of subject are subjected to space travel and the second set of subjects are not subjected to space travel. In some embodiments, the first and second set of subjects are administered an intervention. In some embodiments, the method comprises analyzing a fluorescence intensity spatially across the stained first and second set of one or more biological samples. In some embodiments, the method comprises calculating a set of features of the first and second set of subjects' spatial fluorescent intensity across the stained first and second set of one or more biological samples. In some embodiments, each feature of the first and second set of subjects' exposomic features comprises one or more metrics quantifying temporal dynamics in the fluorescence intensity trace.

In some embodiments, the method comprises outputting a probability of a subject's response to an intervention to reduce the biological effects of space travel as an output of a trained predictive model when the predictive model is provided the subject's exposomic features of spatial fluorescent intensity across a stained one or more biological samples of the subject. In some embodiments, the trained predictive model is trained with the first and second set of subjects' exposomic features' one or more metrics, corresponding administered intervention, and clinical response to the administered intervention.

In some embodiments, the trained predictive model is configured to determine whether a biological sample of the subjects has been dysregulated via space travel.

In some embodiments, the trained predictive model is configured to determine the effect of the intervention as a measure of intervention efficacy based at least in part on the analysis of the fluorescence intensity spatially across one or more biological samples.

In some embodiments, the first or second set of one or more biological samples comprise hair, teeth, toenails, fingernails, physiologic parameters, or any combination thereof.

In some embodiments, the intervention comprises a diet, pharmaceutical therapeutic, or any combination thereof.

In some embodiments, the diet comprises iodine supplementation.

In some embodiments, the intervention ameliorates radiation exposure.

In some embodiments, the first or second set of subjects comprise a human, non-human mammal, or any combination thereof.

In some embodiments, the non-human mammal comprises mice.

In some embodiments, the trained predictive model comprises a gradient-boosted ensemble model.

In some embodiments, a measurement of temporal dynamics of the spatial fluorescence intensity traces include: determination of a linear slope, determination of a plurality of non-linear parameters describing curvature of the one or more exposomic signatures, determination of an abrupt change in intensity of the one or more exposomic signatures, determination of one or more changes in a baseline intensity of the one or more exposomic signatures, determination of a change of a frequency-domain representation of the one or more exposomic signatures, determination of a change of the power-spectral domain representation of the one or more exposomic signatures, determination of one or more recurrence quantification analysis parameters, determination of one or more cross-recurrence quantification analysis parameters, determination of one or more joint recurrence quantification analysis parameters, determination of one or more multidimensional recurrence quantification analysis parameters, estimation of a Lyapunov spectra, determination of a maximum Lyapunov exponent, or any combination thereof.

In some embodiments, the measurement of the temporal dynamics comprises determination of one or more the recurrence quantification analysis parameters, wherein the one or more the recurrence quantification analysis parameters comprises a recurrence rate, a determinism, a mean diagonal length, a maximum diagonal length, a divergence, a Shannon entropy in diagonal length, a trend in recurrence, a laminarity, a trapping time, a maximum vertical line length, a Shannon entropy in vertical line length, a mean recurrence time, a Shannon entropy in recurrence time, or a number of a most probable recurrence.

In some embodiments, the analyzing comprises obtaining a fluorescence image of the first and second set stained one or more biological samples and analyzing the spatial fluorescence intensity of the fluorescence image.

In some embodiments, obtaining the fluorescence image of the stained tooth sample comprises using an inverted or non-inverted confocal microscope.

In some embodiments, staining the first set of one or more biological samples of a first and second set of subjects comprises using a C-reactive stain. In some embodiments, the method further comprises sectioning the tooth sample. In some embodiments, staining the first and second set of one or more biological samples comprises decalcifying the first and second set of one or more biological samples.

FIG. 24 shows quantitative metrics derived from cross-recurrent quantification analysis of signal dyads from control animals housed in a ground facility and mice that traveled to the International Space Station, as described in some embodiments herein. Statistically significant differences in the ion dynamics are observed between ground control cohort and flight cohort of mice, where Ba: Ca dynamic is the most apparent.

In some embodiments, the trained model is selected from the group consisting of: a neural network algorithm, a support vector machine algorithm, a decision tree algorithm, an unsupervised clustering algorithm, a supervised clustering algorithm, a regression algorithm, a gradient-boosting algorithm, and any combination thereof.

In some embodiments, the trained model comprises a gradient-boosted decision tree.

In some embodiments, the trained predictive model comprises a classification algorithm.

In some embodiments, the classification algorithm is trained to identify features in spatial fluorescence intensity associated with the space travel.

In some aspects, the present disclosure describes a method for outputting one or more pathways associated with space travel. In some embodiments, the method comprises receiving one or more biological samples of a first set of one or more subjects and a second set of one or more subjects. In some embodiments, the first set of one or more subjects has participated in space travel and the second set of one or more subjects has not.

In some embodiments, the method comprises determining one or more pathways' exposomic signatures from the one or more biological samples of the first and second set of one or more subjects.

In some embodiments, the method comprises calculating exposomic features of the one or more pathways' exposomic signatures.

In some embodiments, the method comprises outputting the one or more pathways associated with space travel by comparing the first and second set of subjects' exposomic features of the one or more pathways.

In some embodiments, each feature of the exposomic features comprises one or more quantitative metrics.

In some embodiments, space travel comprises traveling beyond the Karman line at an altitude of at least 100 kilometers (km) above the Earth's surface.

In some embodiments, the biological sample comprises hair, teeth, toenails, fingernails, physiologic parameters, or any combination thereof.

In some embodiments, the exposomic features comprise a measurement of temporal dynamics of the one or more exposomic signatures.

In some embodiments, the measurement of the temporal dynamics comprises: determination of a linear slope, determination of a plurality of non-linear parameters describing curvature of the one or more exposomic signatures, determination of an abrupt change in intensity of the one or more exposomic signatures, determination of one or more changes in a baseline intensity of the one or more exposomic signatures, determination of a change of a frequency-domain representation of the one or more exposomic signatures, determination of a change of the power-spectral domain representation of the one or more exposomic signatures, determination of one or more recurrence quantification analysis parameters, determination of one or more cross-recurrence quantification analysis parameters, determination of one or more joint recurrence quantification analysis parameters, determination of one or more multidimensional recurrence quantification analysis parameters, estimation of a Lyapunov spectra, determination of a maximum Lyapunov exponent, or any combination thereof.

In some embodiments, the measurement of the temporal dynamics comprises determination of one or more recurrence quantification analysis parameters, wherein the one or more recurrence quantification analysis parameters comprises a recurrence rate, a determinism, a mean diagonal length, a maximum diagonal length, a divergence, a Shannon entropy in diagonal length, a trend in recurrence, a laminarity, a trapping time, a maximum vertical line length, a Shannon entropy in vertical line length, a mean recurrence time, a Shannon entropy in recurrence time, or a number of a most probable recurrence.

In some embodiments, the exposomic signatures are measured by mass spectrometry, laser ablation-inductively coupled plasma-mass spectrometry, laser induced breakdown spectroscopy, Raman spectroscopy, immunohistochemistry fluorescence, or any combination thereof.

In some embodiments, the exposomic features are derived from one or more attractors.

In some embodiments, the method comprising analyzing a dynamic relationship between the one or more signal recurrence rates, determinism, mean diagonal length, maximum diagonal length, divergence, Shannon entropy in diagonal length, trend in recurrences, laminarity, trapping time, maximum vertical line length, Shannon entropy in vertical line lengths, mean recurrence time, Shannon entropy in recurrence times, number of the most probable recurrences, or any combination thereof.

In some embodiments, the one or more subjects are a human, non-human mammal, vertebrate, or any combination thereof.

In some aspects, the present disclosure describes a method for providing a treatment to ameliorate the effects of space travel. In some embodiments, the method comprises receiving one or more biological samples of one or more subjects participating in space travel. In some embodiments, the method comprises determining one or more pathways' exposomic signatures from the one or more biological samples of the one or more subjects. In some embodiments, the method comprises calculating exposomic features of the one or more pathways' exposomic signatures. In some embodiments, the method comprises providing a treatment to the one or more subjects. In some embodiments, the treatment ameliorates the effect of the space travel on the one or more subjects' exposomic features of the one or more pathways' exposomic signatures.

In some embodiments, the treatment comprises a diet, pharmaceutical, nutraceutical, food bar, or any combination thereof.

In some embodiments, each feature of the exposomic features comprises one or more quantitative metrics.

In some embodiments, the space travel comprises traveling beyond the Karman line at an altitude of at least 100 kilometers (km) above the Earth's surface.

In some embodiments, the biological sample comprises hair, teeth, toenails, fingernails, physiologic parameters, or any combination thereof.

In some embodiments, the exposomic features comprise a measurement of temporal dynamics of the one or more exposomic signatures.

In some embodiments, the measurement of the temporal dynamics comprises: determination of a linear slope, determination of a plurality of non-linear parameters describing curvature of the one or more exposomic signatures, determination of an abrupt change in intensity of the one or more exposomic signatures, determination of one or more changes in a baseline intensity of the one or more exposomic signatures, determination of a change of a frequency-domain representation of the one or more exposomic signatures, determination of a change of the power-spectral domain representation of the one or more exposomic signatures, determination of one or more recurrence quantification analysis parameters, determination of one or more cross-recurrence quantification analysis parameters, determination of one or more joint recurrence quantification analysis parameters, determination of one or more multidimensional recurrence quantification analysis parameters, estimation of a Lyapunov spectra, determination of a maximum Lyapunov exponent, or any combination thereof.

In some embodiments, the measurement of the temporal dynamics comprises determination of one or more recurrence quantification analysis parameters, wherein the one or more recurrence quantification analysis parameters comprises a recurrence rate, a determinism, a mean diagonal length, a maximum diagonal length, a divergence, a Shannon entropy in diagonal length, a trend in recurrence, a laminarity, a trapping time, a maximum vertical line length, a Shannon entropy in vertical line length, a mean recurrence time, a Shannon entropy in recurrence time, or a number of a most probable recurrence.

In some embodiments, the exposomic signatures are measured by mass spectrometry, laser ablation-inductively coupled plasma-mass spectrometry, laser induced breakdown spectroscopy, Raman spectroscopy, immunohistochemistry fluorescence, or any combination thereof.

In some embodiments, the exposomic features are derived from one or more attractors.

In some embodiments, the method further comprises analyzing a dynamic relationship between the one or more signal recurrence rates, determinism, mean diagonal length, maximum diagonal length, divergence, Shannon entropy in diagonal length, trend in recurrences, laminarity, trapping time, maximum vertical line length, Shannon entropy in vertical line lengths, mean recurrence time, Shannon entropy in recurrence times, number of the most probable recurrences, or any combination thereof.

In some embodiments, the one or more subjects are a human, non-human mammal, vertebrate, or any combination thereof.

### EXAMPLES

### Example 1: Acquiring Exposomic Biochemical Signatures from a Biological Sample

Temporal exposome biochemical signatures were acquired from a biological sample using laser ablation-inductively coupled-plasma mass spectrometry (LA-ICP-MS). For this particular example, hair shaft samples were used and chemicals exposome data was acquired. The hair shaft was harvested from subjects and pretreated by washing the sample with one or more solvents and/or surfactants followed by drying. Particularly, the hair shaft sample was washed in TRITON X-100 and ultrapure metal free water (e.g., MILLI-Q water) and dried overnight in an oven (e.g., at 60° C.). The pretreatment further includes preparing the hair shaft for measurement by placing the hair shaft on a glass slide (e.g., a microscopic glass slide) with an adhesive film (e.g., a double-sided tape). The hair shaft is positioned such that the hair shaft is substantially straight. The glass slide with the hair shaft is then placed into a LA-ICP-MS system for analysis.

The analysis commences by the LA-ICP-MS system completing a pre-ablating step where the hair shaft sample is ablated to remove surface debris and/or impurities from the hair shaft. The pre-ablation is performed with a low laser energy, approximately 10% laser energy that only releases particles on the surface of the hair shaft sample but does not release particles from below the surface. The pre-ablation is performed using a laser wavelength of 193 nm and a laser energy below 0.6 J/cm² (e.g., the laser energy is 0.6 J/cm², 0.5 J/cm², 0.4 J/cm², 0.3 J/cm², 0.2 J/cm², or 0.1 J/cm²).

After pre-ablation, the LA-ICP-MS system irradiates the hair shaft sample with a laser energy below 1.8 J/cm² with laser spot size of 10 μm to 30 μm to obtain chemical samples from respective positions along a reference line of the hair shaft sample. Each position along the hair shaft approximates to 2.2 hours or 130 minutes of time of the subject's life. The laser irradiating spatially irradiates the entirety of length of the hair shaft producing particles at a discretion location of the hair shaft that are then ionized with an inductively coupled plasma. The mass spectrometer then analyzes the obtained chemical samples providing the respective chemicals data read-out of what chemicals are present in what quantities at a given spatial location. This process is repeated, and chemicals data is collected sequentially at a plurality of positions along the hair shaft from the hair shaft root to the tip of the shaft furthest away from the root. Positional data, indicating time, and the respective isotopes present at each location of the hair shaft are correlated for further analysis.

The output data is further analyzed and processed by spike removal that removes extreme peaks in the exposome biochemical signature and smoothens the data. Outliers are identified by calculating the mean absolute difference between each adjacent measurement along the hair shaft. Values indicating a mean absolute difference from the preceding point exceeds three standard deviations of the mean are flagged as outliers. These outlier values are then replaced with a moving median filter, which calculates a running median value of the original exposome biochemical signature in bins of 10 adjacent data points.

Processed data can then be used in various bioinformation analysis methods that identify the effects of clinical intervention on elemental signal intensities and signal dynamics.

### Example 2: Dynamic Molecular Profiles in Tooth Samples for Determining Disease Risk

Using methods and systems of the present disclosure, molecular profiles in tooth samples were generated and subsequently analyzed to determine a disease risk in a subject. Generally, the temporal dynamics of biological response (e.g., inflammation) were found to be imprinted in samples (e.g., tooth samples), and can be analyzed to determine disease risk in a subject. Dynamic molecular profiles were generated for C-reactive protein (CRP), which is a marker of inflammation. Using the tooth biomarkers, dynamic time-series profiles of CRP and inflammation were generated during a time period that comprised fetal (prenatal) development and early childhood in two sets of children-a first set with autism spectrum disorder (37 cases) and a second set without autism spectrum disorder (77 controls). The time-series CRP profiles were analyzed to reveal novel features of the dynamics of the CRP signal, which accurately distinguished the autism cases from controls. For example, the inflammation profiles that were present before age of 1 year were highly differential between cases and controls. In comparison, a clinical diagnosis of autism is usually determined around the age of 3 to 4 years.

A primary tooth sample was obtained from each child subject. The teeth samples were sectioned open, decalcified and an immunohistochemistry stain (e.g., dentine) was applied to the teeth samples. The immunohistochemistry stain effectively mapped C-reactive protein (a molecular marker of inflammation) along the growth rings of the teeth samples in order to develop temporal profiles of inflammation over the prenatal and postnatal period. The temporal profiles were analyzed using machine learning algorithms of the present disclosure to train highly accurate classifiers to determine disease risk (e.g., autism).

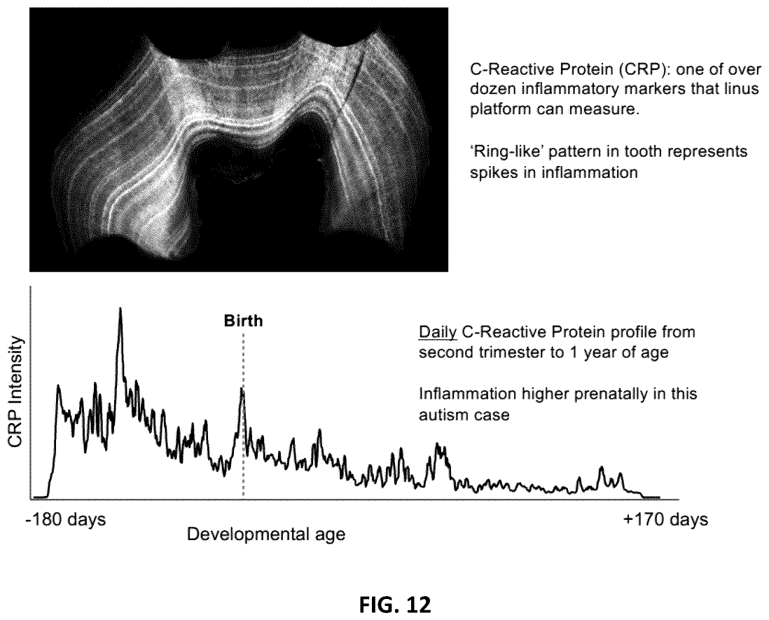

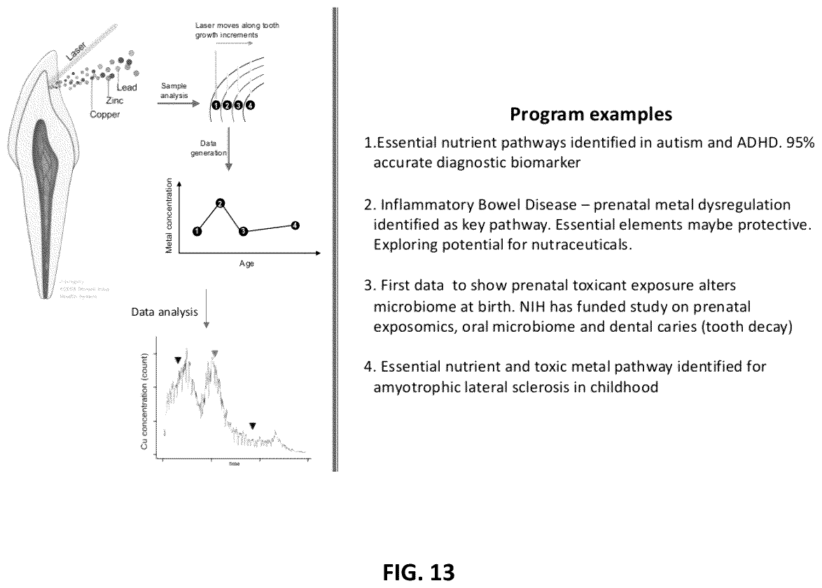

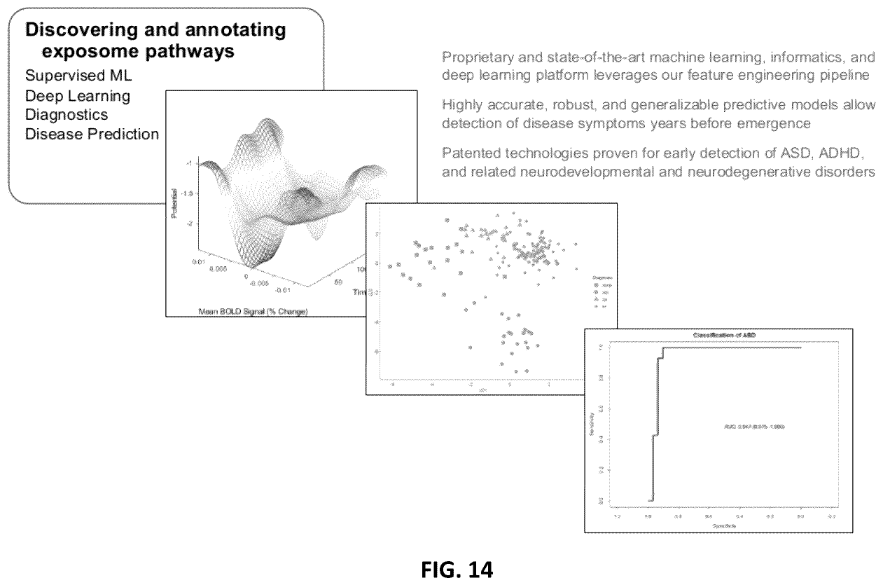

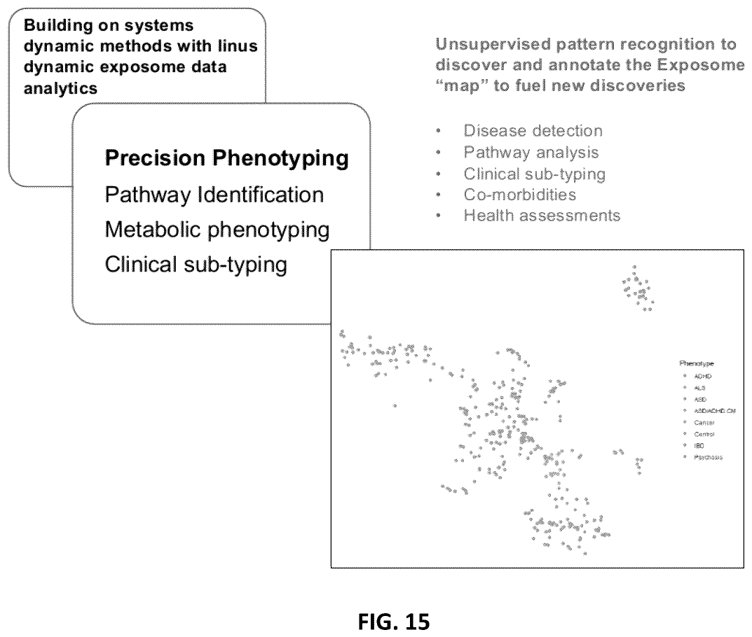

FIG. 12 shows an example of a daily C-reactive protein profile of a subject over time, where the y-axis is indicative of CRP intensity, and the x-axis is indicative of developmental age. The developmental age of the child subject included a time period ranging from the second trimester of gestation (e.g., starting at 140 days before birth, when the subject was in the prenatal stage) to about 6 months of age. As shown in FIG. 12, inflammation (as indicated by CRP intensity) profile of a child with autism with high CRP intensity prenatally.

### Example 3: Detection of Space Travel Signatures in Animal Model Hair Samples

A between-subjects experimental study was conducted with mice housed in a ground facility (N=10) and mice that traveled to the International Space Station (ISS; N=10). Hair (whisker) samples were collected for analysis, as described by the methods and systems described elsewhere herein. The hair samples were analyzed to quantify signal dynamics for multiple elements that were then compared these between experimental conditions. The representative data of the signal dynamics for multiple elements seen in FIG. 24. This approach revealed several significant differences in elemental dynamics between mice that experienced space travel and mice that did not, including perturbation of barium: calcium dynamics (P=0.02), manganese: iodine dynamics (P=0.04), and zinc: iron (P=0.002) dynamics. These patterns of dysregulation constitute a detectable signature of space travel, which might serve as a biomarker of space flight or as a target of ameliorative interventions.

### Example 4: Detection of Space Travel Signatures in Human Hair Samples

Human hair sample elemental dynamics of individuals that traveled to space via commercial space flight initiatives (Blue Origin) was analyzed by the methods and systems described elsewhere herein. Dynamics of the longitudinal signal intensity of several elemental pathways were quantified. In particularly, the elemental pathways identified from the animal model studies as being dysregulated by space travel, including barium, manganese, and zinc were analyzed. In FIG. 25, a time-varying (expressed in days) magnitude of these signals relative to the timing of space flight, with the day of space flight identified as day 0 can be observed. Critically, and consistently with the patterns observed in animal model studies of Example 3, we identified a notable change in barium, manganese, and zinc dynamics following space flight. This pattern of change provides a signature of spaceflight which might be used as a biomarker of stress or a target in ameliorative efforts.

### Example 5: Detection of Ameliorative Treatments in Animal Model Hair Samples

A between-subjects experimental study with mice housed in a ground facility (N=20) were conducted to determine the effect of space flight ameliorative treatments. In this particular study, one group (N=10) of animals was provided iodine-supplementation (4 ppm added to water supply) and a second group (N=10) of animal was not provided iodine-supplementation. From each group, we collected hair (whisker) samples for analysis with the methods and systems, described elsewhere herein. In FIG. 26, it can be seen that that iodine supplementation significantly reduced levels of barium: calcium (P=0.01) and zinc: iron (P=0.0001) dynamics. Critically, given prior findings (see Example 1) which showed that space travel reduced levels of these signatures, these findings highlight how these methods can be used to demonstrate the effects of ameliorative treatments, and link the action of ameliorative effects to a molecular mechanism and/or effector.

### Example 6: Detection of Dietary Effects on Signatures of Space Travel

A between-subjects experimental study with mice housed in a ground facility (N=20) was conducted to determine dietary effects on signatures of space travel. In this study, one group of mice (N=10) were reared standard rodent chow diet, while a second group (N=10) were reared with rodent Food bars. From each group, we collected hair (whisker) samples for analysis with the methods and systems, described elsewhere herein. From FIG. 27, it can be seen that dietary manipulation yielded significant changes in levels of barium: calcium (P=0.004) and zinc: iron (P=0.02) dynamics. Critically, given prior findings (see Example 1) which showed that space travel reduced levels of these signatures, these findings highlight how these methods can be used to demonstrate the effects of ameliorative treatments, particularly nutritional interventions; and can link the action of ameliorative effects to a molecular mechanism and/or effector.

### Embodiments

Embodiment 1. A computer-implemented exposomics system, the system comprising: (a) an exposome biochemical signatures database (EDB), the EDB comprising exposomic features for a plurality of subjects that have and have not participated in space travel; and (b) an intervention outcome database (IODB), the IODB comprising information on intervention outcome information for at least one phase of at least one intervention; (c) computer processor comprising: (i) an association software module communicatively coupled to the EDB and the IODB, wherein the association software module is programmed to determine an association between the exposomic features, the clinical phenotype information, and the intervention outcome information for at least one of the plurality of subjects, and (ii) a recommendation software module communicatively coupled to the EDB and the IODB, the recommendation software module programmed to provide an intervention recommendation for the at least one of the plurality of subjects based at least in part on the exposomic features, the clinical phenotype information, the intervention outcome information, and the association between the exposomic features, the clinical phenotype information, and the intervention outcome information for the at least one of the plurality of subjects.

Embodiment 2. The system of embodiment 1, further comprising a clinical database (CDB), the CDB comprising clinical phenotype information for the plurality of subjects.

Embodiment 3. The system of embodiment 1, wherein the exposome biochemical signatures comprises at least 100, at least 1,000, or at least 10,000 distinct exposomic biochemical signatures.

Embodiment 4. The system of embodiment 1, wherein the intervention outcome information comprises classifications of non-responder, adverse responder, and positive responder for the at least one intervention.

Embodiment 5. The system of embodiment 1, wherein the intervention outcome comprises one or more inclusion criteria or exclusion criteria for the at least one intervention.

Embodiment 6. The system of embodiment 1, wherein the exposome biochemical signatures are obtained by assaying biological samples of the plurality of subjects.

Embodiment 7. The system of embodiment 6, wherein the biological samples comprise tooth samples, nail samples, hair samples, physiologic parameter, or any combination thereof.

Embodiment 8. The system of embodiment 6, wherein the assaying comprises obtaining mass spectrometry measurements, laser ablation-inductively coupled plasma mass spectrometry measurements, laser induced breakdown spectroscopy measurements, Raman spectroscopy measurements, immunohistochemistry measurements, or any combination thereof.

Embodiment 9. The system of embodiment 8, wherein the mass spectrometry measurements comprise measurements of one or more chemicals.

Embodiment 10. The system of embodiment 9, wherein the one or more chemicals comprise aluminum, arsenic, barium, bismuth, calcium, copper, iodide, lead, lithium, magnesium, manganese, phosphorus, sulfur, tin, strontium, zinc, or any combination thereof.

Embodiment 11. The system of embodiment 1, wherein the exposomic features comprises features of the dynamic temporal biochemical responses of the plurality of subjects.

Embodiment 12. The system of embodiment 1, wherein the corresponding plurality of exposomic features for a corresponding subject in the plurality of subjects comprises one or more fluorescence images of one or more biological samples of the corresponding subject.

Embodiment 13. The system of embodiment 1, wherein the corresponding plurality of exposomic features for a corresponding subject in the plurality of subjects comprises one or more spatial maps of one or more Raman spectra of a biological sample of the corresponding subject.

Embodiment 14. The system of embodiment 1, wherein the exposome biochemical signatures are associated with space travel.

Embodiment 15. The system of embodiment 14, wherein the at least one program further comprises instructions for analyzing the corresponding plurality of exposomic features using a trained model to determine an association with space travel

Embodiment 16. The system of embodiment 15, wherein the exposomic features are analyzed using a trained classifier to determine the association with the space travel.

Embodiment 17. The system of embodiment 16, wherein the trained classifier is selected from the group consisting of: a neural network algorithm, a support vector machine algorithm, a decision tree algorithm, an unsupervised clustering algorithm, a supervised clustering algorithm, a regression algorithm, a gradient-boosting algorithm, and any combination thereof.

Embodiment 18. A method for selecting a subject for an intervention, the method comprising: (a) providing a trained predictive model, wherein the trained predictive model is trained on one or more subjects' clinical metadata, exposomic features, and corresponding intervention outcome information; (b) detecting a biochemical signature obtained from a biological sample from a subject seeking the intervention, thereby producing prospective exposomic features; (c) predicting, with the trained predictive model, the predicted intervention outcome information of the subject seeking the intervention, wherein exposomic features and clinical meta of the subject seeking the intervention are inputs to the trained predictive model; and (d) selecting the subject for the intervention or excluding the subject from the intervention, based at least in part on the predicted intervention outcome information of the subject.

Embodiment 19. The method of embodiment 18, wherein the biochemical signature is obtained by assaying a biological sample of the subject.

Embodiment 20. The method of embodiment 19, wherein the biological sample comprises a tooth sample, a nail sample, a hair sample, or any combination thereof.

Embodiment 21. The method of embodiment 19, wherein the assaying comprises collecting data from laser ablation-inductively coupled plasma mass spectrometry measurements, laser induced breakdown spectroscopy measurements, Raman spectroscopy measurements, immunohistochemistry measurements, or any combination thereof.

Embodiment 22. The method of embodiment 21, wherein the laser ablation-inductively coupled plasma mass spectrometry measurements comprise measurements of one or more element chemicals.

Embodiment 23. The method of embodiment 22, wherein the one or more element chemicals comprise aluminum, arsenic, barium, bismuth, calcium, copper, iodide, lead, lithium, magnesium, manganese, phosphorus, sulfur, tin, strontium, zinc, or any combination thereof.

Embodiment 24. The method of embodiment 18, wherein the biochemical signature comprises fluorescence images of the biological sample.

Embodiment 25. The method of embodiment 18, wherein the biochemical signature comprises spatial maps of Raman spectra of the biological sample.

Embodiment 26. The method of embodiment 18, wherein the biochemical signature is associated with space travel.

Embodiment 27. The method of embodiment 18, wherein the intervention comprises a diet, pharmaceutical therapeutic, or any combination thereof.

Embodiment 28. The method of embodiment 18, wherein the trained predictive model is selected from the group consisting of: a neural network algorithm, a support vector machine algorithm, a decision tree algorithm, an unsupervised clustering algorithm, a supervised clustering algorithm, a regression algorithm, a gradient-boosting algorithm, and any combination thereof.

Embodiment 29. The method of embodiment 18, further comprising enrolling the subject into the intervention, when the subject is selected for the intervention.

Embodiment 30. The method of embodiment 18, further comprising evaluating the subject for another intervention, when the subject is excluded from the intervention.

Embodiment 31. A method of selecting an optimal treatment for ameliorating negative effects of space travel, comprising: (a) detecting one or more biochemical signatures obtained from one or more biological sample from one or more subjects that have not participated in space travel, thereby producing one or more reference exposomic features; (b) detecting features of one or more biochemical signature obtained from one or more biological sample from one or more subjects that have participated in space travel, thereby producing one or more pre-treatment exposomic features; (c) administering a treatment to the one or more subjects that have participated in space travel; (d) detecting features of one or more biochemical signatures obtained from one or more biological sample from one or more subjects that have participated in space travel after a period of time has elapsed after receiving the treatment, thereby producing one or more post-treatment exposomic features; (e) determining a difference between the one or more reference exposomic features of the one or more subjects that have not participated in space travel, the one or more pre-treatment exposomic features of the one or more subjects that have participated in space travel, and the one or more post-treatment exposomic features of the one or more subjects that have participated in space travel; and (f) selecting one or more optimal treatments based at least in part on the determined difference between the one or more reference exposomic features, one or more pre-treatment exposomic features, and one or more post-treatment exposomic features, wherein the one or more optimal treatments are selected based on the determined differences satisfying a pre-determined criterion.

Embodiment 32. The method of embodiment 31, wherein the optimal treatment may comprise a pharmaceutical, nutraceutical, or any combination thereof.

Embodiment 33. The method of embodiment 31, wherein the pre-determined criterion comprises a difference between the one or more pre-treatment exposomic features and the one or more post-treatment exposomic features to the one or more reference exposomic features.

Embodiment 34. The method of embodiment 33, wherein the period of time comprises at least about 1 hour, at least about 1 day, at least about 1 week, at least about 1 month, at least about 1 year, or any combination thereof.

Embodiment 35. The method of embodiment 33, wherein the difference comprises a change of at least 10% of the one or more post-treatment exposomic features toward the one or more reference exposomic features.

Embodiment 36. The method of embodiment 31, wherein the pre-treatment exposomic features, post-treatment exposomic features, or any combination thereof is obtained by assaying a biological sample of the subject.

Embodiment 37. The method of embodiment 36, wherein the biological sample comprises a tooth sample, a nail sample, a hair sample, a physiologic parameter, or any combination thereof.

Embodiment 38. The method of embodiment 36, wherein the assaying comprises obtaining laser ablation-inductively coupled plasma mass spectrometry measurements, laser induced breakdown spectroscopy measurements, Raman spectroscopy measurements, immunohistochemistry measurements, or any combination thereof.

Embodiment 39. The method of embodiment 38, wherein the laser ablation-inductively coupled plasma mass spectrometry comprises measurements of one or more element chemicals.

Embodiment 40. The method of embodiment 39, wherein the one or more element chemicals comprise aluminum, arsenic, barium, bismuth, calcium, copper, iodide, lead, lithium, magnesium, manganese, phosphorus, sulfur, tin, strontium, zinc, or any combination thereof.

Embodiment 41. The method of embodiment 31, wherein the biochemical signature comprises fluorescence images of the biological sample.

Embodiment 42. The method of embodiment 31, wherein the biochemical signature comprises spatial maps of Raman spectra of the biological sample.

Embodiment 43. The method of embodiment 38, wherein the immunohistochemistry measurements comprise measurements of c-reactive stain.

Embodiment 44. The method of embodiment 31, wherein the differences between the reference exposomic features, pre-treatment exposomic features, post-treatment exposomic features, or any combination thereof is analyzed using a trained classifier.

Embodiment 45. The method of embodiment 44, wherein the trained classifier is selected from the group consisting of: a neural network algorithm, a support vector machine algorithm, a decision tree algorithm, an unsupervised clustering algorithm, a supervised clustering algorithm, a regression algorithm, a gradient-boosting algorithm, and any combination thereof.

Embodiment 46. A method for evaluating the effects of an intervention in a plurality of subjects, the method comprising: sampling, for each respective subject in the plurality of subjects, each respective position in a plurality of positions along a reference line on a corresponding biological sample associated with chemical dynamics of the respective subject, thereby obtaining a corresponding plurality of chemical samples for the respective subject, each chemical sample in the plurality of chemical samples corresponding to a different position in the plurality of positions, and each position in the plurality of positions representing a different period of growth of the corresponding biological sample associated with chemical dynamics, wherein the plurality of positions comprises: one or more positions representing a period of growth prior to the intervention, one or more positions representing a period of growth during the intervention, and one or more positions representing a period of growth after the intervention; analyzing, for each respective subject in the plurality of subjects, the corresponding plurality of chemical samples for the respective subject with a mass spectrometer thereby obtaining a corresponding first dataset that includes a plurality of traces, each trace in the plurality of traces being a concentration of a corresponding chemicals, in a plurality of chemicals, over time collectively determined from the plurality of chemical samples, generating, for each of one or more chemicals in the plurality of chemicals, a corresponding isotope data set comprising: a set of pre-intervention features corresponding to the time period of biological sample growth prior to the intervention, the set of pre-intervention features comprising, for each respective subject in the plurality of subjects, one or more features derived from the concentration of the respective chemicals measured from the one or more positions representing a period of growth prior to the intervention, a set of intervention features corresponding to the time period of biological sample growth during the intervention, the set of intervention features comprising, for each respective subject in the plurality of subjects, one or more features derived from the concentration of the respective chemicals measured from the one or more positions representing a period of growth during the intervention, and a set of post-intervention features corresponding to the time period of biological sample growth after the intervention, the set of post-intervention features comprising, for each respective subject in the plurality of subjects, one or more features derived from the concentration of the respective chemicals measured from the one or more positions representing a period of growth after the intervention; and evaluating changes in chemical dynamics in response to the intervention using, for each chemicals in the one or more chemicals, the corresponding isotope data set.

Embodiment 47. The method of embodiment 46, wherein the evaluating comprises performing, for each of the one or more chemicals, a probabilistic hypothesis test using (i) the set of pre-intervention features and (ii) one or both of the set of intervention features and the set of post-intervention features.

Embodiment 48. A method for evaluating the effects of an intervention in a plurality of subjects, the method comprising: sampling, for each respective subject in the plurality of subjects, each respective position in a plurality of positions along a reference line on a corresponding biological sample associated with chemical dynamics of the respective subject, thereby obtaining a corresponding plurality of chemical samples for the respective subject, each chemical sample in the plurality of chemical samples corresponding to a different position in the plurality of positions, and each position in the plurality of positions representing a different period of growth of the corresponding biological sample associated with chemical dynamics, wherein the plurality of positions comprises: one or more positions representing a period of growth prior to the intervention, one or more positions representing a period of growth during the intervention, and one or more positions representing a period of growth after the intervention; analyzing, for each respective subject in the plurality of subjects, the corresponding plurality of chemical samples for the respective subject with a mass spectrometer thereby obtaining a corresponding first dataset that includes a plurality of traces, each trace in the plurality of traces being a concentration of a corresponding chemicals, in a plurality of chemicals, over time collectively determined from the plurality of chemical samples, generating, for a set of two or more chemicals in the plurality of chemicals, a respective aggregate isotope data set comprising: a set of pre-intervention features corresponding to biological sample growth prior to the intervention, the set of pre-intervention features comprising values for one or more dimension reduction components formed from features derived from, for each respective subject in the plurality of subjects, concentrations of each of the two or more chemicals measured from the one or more positions representing a period of growth prior to the intervention, a set of intervention features corresponding to biological sample growth during the intervention, the set of intervention features comprising values for one or more dimension reduction components formed from features derived from, for each respective subject in the plurality of subjects, concentrations of each of the two or more chemicals measured from the one or more positions representing a period of growth during the intervention, and a set of post-intervention features corresponding to biological sample growth after the intervention, the set of post-intervention features comprising values for one or more dimension reduction components formed from features derived from, for each respective subject in the plurality of subjects, concentrations of each of the two or more chemicals measured from the one or more positions representing a period of growth after the intervention; and evaluating changes in chemical dynamics in response to the intervention using the aggregate isotope data set.

Embodiment 49. The method of embodiment 48, wherein the evaluating comprises performing a probabilistic hypothesis test using (i) the set of pre-intervention features and (ii) one or both of the set of intervention features and the set of post-intervention features.

Embodiment 50. The method of embodiment 48 or 49, wherein, for each of the set of pre-intervention features, the set of intervention features, and the set of post-intervention features, the values for the dimension reduction components are each determined from the features derived from the features of each of the two or more chemicals measured from a single respective subject in the plurality of subjects.

Embodiment 51. The method of embodiment 48 or 49, wherein, for each of the set of pre-intervention features, the set of intervention features, and the set of post-intervention features, the values for the dimension reduction components are each determined from an aggregate of the features derived from the features of each of the two or more chemicals measured from a plurality of respective subjects in the plurality of subjects.

Embodiment 52. The method of any one of embodiments 46-51, wherein the one or more features derived from the concentration of the respective chemicals measured from the one or more positions representing a period of growth are the concentrations, a normalized concentration thereof, or related descriptive statistics or derived parameters thereof.

Embodiment 53. The method of any one of embodiments 46-51, wherein the one or more features derived from the concentration of the respective chemicals measured from the one or more positions representing a period of growth are selected from the group consisting of recurrence rates, determinism, mean diagonal length, maximum diagonal length, divergence, Shannon entropy in diagonal length, trend in recurrences, laminarity, trapping time, maximum vertical line length, Shannon entropy in vertical line lengths, mean recurrence time, Shannon entropy in recurrence times, and number of the most probable recurrences; these measures are derived through the application of recurrence quantification analysis parameters, cross-recurrence quantification analysis parameters, joint recurrence quantification analysis parameters, and/or multidimensional recurrence quantification analysis.

Embodiment 54. A method for evaluating the effects of an intervention in a plurality of subjects, the method comprising: sampling, for each respective subject in the plurality of subjects, each respective position in a plurality of positions along a reference line on a corresponding biological sample associated with chemical dynamics of the respective subject, thereby obtaining a corresponding plurality of chemical samples for the respective subject, each chemical sample in the plurality of chemical samples corresponding to a different position in the plurality of positions, and each position in the plurality of positions representing a different period of growth of the corresponding biological sample associated with chemical dynamics, wherein the plurality of positions comprises: one or more positions representing a period of growth prior to the intervention, one or more positions representing a period of growth during the intervention, and one or more positions representing a period of growth after the intervention; analyzing, for each respective subject in the plurality of subjects, the corresponding plurality of chemical samples for the respective subject with a mass spectrometer thereby obtaining a corresponding first dataset that includes a plurality of traces, each trace in the plurality of traces being a concentration of a corresponding chemicals, in a plurality of chemicals, over time collectively determined from the plurality of chemical samples, applying, for each respective subject in the plurality of subjects and for each of one or more chemicals in the plurality of chemicals, a distributed lag model or similar non-linear distribution model as a function of time relative to the intervention to the concentration of the respective chemicals measured from the plurality of positions, or a feature derived therefrom, to generate a corresponding contribution data set representing the contribution of the intervention to the concentration of the respective chemicals in the respective subject as a function of time; and evaluating changes in chemical dynamics in response to the intervention using the corresponding contribution data set, for each of the one or more chemicals, from each respective subject in the plurality of subjects.

Embodiment 55. A method for evaluating the effects of an intervention in a plurality of subjects, the method comprising: sampling, for each respective subject in the plurality of subjects, each respective position in a plurality of positions along a reference line on a corresponding biological sample associated with chemical dynamics of the respective subject, thereby obtaining a corresponding plurality of chemical samples for the respective subject, each chemical sample in the plurality of chemical samples corresponding to a different position in the plurality of positions, and each position in the plurality of positions representing a different period of growth of the corresponding biological sample associated with chemical dynamics, wherein the plurality of positions comprises: one or more positions representing a period of growth prior to the intervention, one or more positions representing a period of growth during the intervention, and one or more positions representing a period of growth after the intervention; analyzing, for each respective subject in the plurality of subjects, the corresponding plurality of chemical samples for the respective subject with a mass spectrometer thereby obtaining a corresponding first dataset that includes a plurality of traces, each trace in the plurality of traces being a concentration of a corresponding chemicals, in a plurality of chemicals, over time collectively determined from the plurality of chemical samples, generating, for each respective subject in the plurality of subjects and for a set of two or more chemicals in the plurality of chemicals, a corresponding aggregate isotope data set comprising a set of features comprising values for one or more dimension reduction components formed from features derived from concentrations of each of the two or more chemicals measured across the different periods of growth for the respective subject; applying, for each respective subject in the plurality of subjects, a distributed lag model or similar non-linear distribution model as a function of time relative to the intervention to the corresponding aggregate isotope data set to generate a corresponding contribution data set representing the contribution of the intervention to the concentration of the set of two or more chemicals in the respective subject as a function of time; and evaluating changes in chemical dynamics in response to the intervention using the corresponding contribution data for each respective subject in the plurality of subjects.

Embodiment 56. The method of any one of embodiments 46-55, wherein the intervention is ingestion of a nutraceutical composition.

Embodiment 57. The method of embodiment 56, further comprising, in response to the evaluating changes in chemical dynamics, altering the composition of the nutraceutical composition to adjust the effects of the ingestion of the nutraceutical composition.

Embodiment 58. The method of embodiment 56, further comprising, in response to the evaluating changes in chemical dynamics, supplementing ingestion of the nutraceutical composition with ingestion of one or more dietary supplements.

Embodiment 59. The method of any one of embodiments 46-58, further comprising evaluating changes in the metabolism of one or more additional metabolites in response to the intervention.

Embodiment 60. The method of embodiment 59, wherein the one or additional metabolites are selected from the group consisting of a perfluoro compound, a paraben, a phthalate, a lipid, an amino acid, an amino acid derivative, and a peptide.

Embodiment 61. A method for outputting one or more quantitative metrics of a subject's one or more exposomic signatures, comprising: (a) receiving a biological sample from a subject; (b) determining one or more exposomic signatures from the biological sample of the subject; (c) calculating one or more features of the one or more exposomic signatures, wherein each feature of the one or more features comprises one or more quantitative metrics; and (d) outputting the one or more quantitative metrics of the one or more features of the subject.

Embodiment 62. The method of embodiment 61, further comprising outputting a health outcome of the subject based at least in part on an association of normalized scores of the subject's one or more features to normalized scores of a second set of subjects' one or more features' one or more quantitative metrics.

Embodiment 63. The method of embodiment 62, wherein the second set of subjects' one or more features' one or more quantitative metrics are stored in a database, wherein the database is a local server or a cloud-based server.

Embodiment 64. The method of embodiment 62, wherein the health outcome comprises a space travel classification, clinical subtype, non-clinical subgrouping related to physiology, anthropometric indicators, behavior indicators, socioeconomic indicators, body mass index, intelligence quotient, socio-economic status, or any combination thereof.

Embodiment 65. The method of embodiment 61, wherein the one or more features comprise a measurement of temporal dynamics of the one or more exposomic signatures.

Embodiment 66. The method of embodiment 65, wherein the measurement of the temporal dynamics comprises: linear slope, non-linear parameters describing curvature of the one or more exposomic signatures, abrupt changes in intensity of the one or more exposomic signatures, changes in baseline intensity of the one or more exposomic signatures, changes of the frequency-domain representation of the one or more exposomic signatures, changes of the power-spectral domain representation of the one or more exposomic signatures, recurrence quantification analysis parameters, cross-recurrence quantification analysis parameters, joint recurrence quantification analysis parameters, multidimensional recurrence quantification analysis parameters, estimation of the Lyapunov spectra, maximum Lyapunov exponent, laminarity, entropy, trapping time (TT), mean diagonal length (MDL), recurrence time (RT), Vmax, determinism, maximum diagonal length, divergence, Shannon entropy in diagonal length, trend in recurrence, maximum vertical line length, Shannon entropy in vertical line length, a mean recurrence time, Shannon entropy in recurrence time, a number of a most probable recurrence, or any combination thereof.

Embodiment 67. The method of embodiment 61, wherein the subject comprises a plurality of subjects.

Embodiment 68. The method of embodiment 61, wherein the one or more features of the one or more signatures comprise phenotypic features, wherein the phenotypic features comprise: electrocardiogram (ECG), electroencephalography, magnetic resonance imaging (MRI), functional magnetic resonance imaging (fMRI), positron emission tomography (PET), genomic, epigenomic, transcriptomic, proteomic, metabolomic, or any combination thereof data.

Embodiment 69. The method of embodiment 61, wherein the one or more features are derived from one or more attractors.

Embodiment 70. The method of embodiment 61, wherein the one or more exposomic signatures are measured by mass spectrometry, laser ablation-inductively coupled plasma mass spectrometry, laser induced breakdown spectroscopy, Raman spectroscopy, immunohistochemistry fluorescence, or any combination thereof.

Embodiment 71. The method of embodiment 61, wherein the biological sample comprises hair, teeth, toenails, fingernails, physiologic parameters, or any combination thereof.

Embodiment 72. The method of embodiment 68, wherein the phenotypic features comprise molecular phenotypes.

Embodiment 73. The method of embodiment 72, wherein the molecular phenotypes are determined by unsupervised analysis, wherein unsupervised analysis comprises clustering, dimensionality-reduction, factor analysis, stacked autoencoding, or any combination thereof.

Embodiment 74. The method of embodiment 66, wherein the recurrence quantification analysis parameters comprise recurrence rates, determinism, mean diagonal length, maximum diagonal length, divergence, Shannon entropy in diagonal length, trend in recurrences, laminarity, trapping time, maximum vertical line length, Shannon entropy in vertical line lengths, mean recurrence time, Shannon entropy in recurrence times, and number of the most probable recurrences.

Embodiment 75. The method of embodiment 69, further comprising analyzing the one or more attractors by potential energy analysis thereby producing a potential energy data space.

Embodiment 76. The method of embodiment 61, wherein the subject's one or more exposomic signatures comprises retrospective exposomic data.

Embodiment 77. The method of embodiment 69, further comprising analyzing a dynamic relationship between the one or more attractors' signal recurrence rates, determinism, mean diagonal length, maximum diagonal length, divergence, Shannon entropy in diagonal length, trend in recurrences, laminarity, trapping time, maximum vertical line length, Shannon entropy in vertical line lengths, mean recurrence time, Shannon entropy in recurrence times, number of the most probable recurrences, or any combination thereof.

Embodiment 78. The method of embodiment 77, wherein the dynamic relationship is determined by cross-convergent mapping (CCM).

Embodiment 79. The method of embodiment 61, further comprising reducing the one or more exposomic signatures to a reduced one or more exposomic signatures.

Embodiment 80. The method of embodiment 69, further comprising constructing a network of the one or more attractors based on similarity of the one or more attractors' temporal exposomic data signal recurrence rates, determinism, mean diagonal length, maximum diagonal length, divergence, Shannon entropy in diagonal length, trend in recurrences, laminarity, trapping time, maximum vertical line length, Shannon entropy in vertical line lengths, mean recurrence time, Shannon entropy in recurrence times, number of the most probable recurrences, or any combination thereof.

Embodiment 81. The method of embodiment 80, further comprising analyzing one or more features of the network of the one or more attractors to determine network connectivity, efficiency, feature importance, pathway importance, related graph-theory based metrics, or any combination thereof.

Embodiment 82. A method for outputting a prediction of one or more subjects' phenotypic data, comprising: (a) receiving one or more biological samples and phenotypic data from a first set of subjects; (b) determining a first set of exposomic signatures from the first set of subjects' one or more biological samples; (c) calculating a first set of features of the first set of exposomic signatures; (d) training a predictive model with the first set of features and the phenotypic data of the first set of subjects; (e) receiving one or more biological samples from a second set of subjects different than the first set of subjects; (f) determining a second set of exposomic signatures from the one or more biological samples of the second set of subjects; (g) calculating a second set of features from the second set of exposomic signatures; and (h) outputting the prediction of the second set of subjects' phenotypic data determined by inputting the second set of features into the trained predictive model.

Embodiment 83. The method of embodiment 82, wherein the first and second set of features comprise one or more quantitative metrics.

Embodiment 84. The method of embodiment 82, wherein the one or more quantitative metrics comprise a measurement of temporal dynamics of the one or more exposomic signatures.

Embodiment 85. The method of embodiment 83, wherein the measurement of the temporal dynamics comprises: linear slope, non-linear parameters describing curvature of the first and second set of exposomic signatures, abrupt changes in intensity of the first and second set of exposomic signatures, changes in baseline intensity of the first and second set of exposomic signatures, changes of the frequency-domain representation of the first and second set of exposomic signatures, changes of the power-spectral domain representation of the first and second set of exposomic signatures, recurrence quantification analysis parameters, cross-recurrence quantification analysis parameters, joint recurrence quantification analysis parameters, multidimensional recurrence quantification analysis parameters, estimation of the Lyapunov spectra or maximum Lyapunov exponent, or any combination thereof.

Embodiment 86. The method of embodiment 82, wherein the first set of features of the first set of exposomic signatures comprises a first set of phenotypic features, and wherein the second set of features of the second set of exposomic signatures comprises a second set of phenotypic features.

Embodiment 87. The method of embodiment 86, wherein the first set of phenotypic features or the second set of phenotypic features comprises: electrocardiogram (ECG), electroencephalography, magnetic resonance imaging (MRI), functional magnetic resonance imaging (fMRI), positron emission tomography (PET), genomic, epigenomic, transcriptomic, proteomic, metabolomic, or any combination thereof data.

Embodiment 88. The method of embodiment 82, wherein the first and second set of features are represented as or derived from one or more attractors.

Embodiment 89. The method of embodiment 88, wherein the one or more attractors are a limit cycle attractor, bistable attractor, or any combination thereof.

Embodiment 90. The method of embodiment 82, wherein the first and second set of exposomic signatures are measured by mass spectrometry, laser ablation-inductively coupled plasma mass spectrometry, laser induced breakdown spectroscopy, Raman spectroscopy, immunohistochemistry fluorescence, or any combination thereof.

Embodiment 91. The method of embodiment 82, wherein the one or more biological samples of the first and second subjects comprise hair, teeth, toenails, fingernails, physiologic parameters, or any combination thereof.

Embodiment 92. The method of embodiment 86, wherein the first set of phenotypic features or the second set of phenotypic features comprise molecular phenotypes.

Embodiment 93. The method of embodiment 92, wherein the molecular phenotypes are determined by unsupervised analysis, wherein unsupervised analysis comprises clustering, dimensionality-reduction, factor analysis, stacked autoencoding, or any combination thereof.

Embodiment 94. The method of embodiment 85, wherein the recurrence quantification analysis parameters comprise recurrence rates, determinism, mean diagonal length, maximum diagonal length, divergence, Shannon entropy in diagonal length, trend in recurrences, laminarity, trapping time, maximum vertical line length, Shannon entropy in vertical line lengths, mean recurrence time, Shannon entropy in recurrence times, and number of the most probable recurrences, or any combination thereof.

Embodiment 95. The method of embodiment 88, further comprising analyzing the one or more attractor by potential energy analysis thereby producing a potential energy data space.

Embodiment 96. The method of embodiment 82, wherein the first and second set of exposomic signatures comprises retrospective, prospective, or any combination thereof dynamic exposomic data.

Embodiment 97. The method of embodiment 88, further comprising analyzing a dynamic relationship between the one or more attractors' recurrence rates, determinism, mean diagonal length, maximum diagonal length, divergence, Shannon entropy in diagonal length, trend in recurrences, laminarity, trapping time, maximum vertical line length, Shannon entropy in vertical line lengths, mean recurrence time, Shannon entropy in recurrence times, and number of the most probable recurrences, or any combination.

Embodiment 98. The method of embodiment 97, wherein the dynamic relationship is determined by cross-convergent mapping (CCM).

Embodiment 99. The method of embodiment 88, further comprising constructing a network of the one or more attractors based on similarity of the one or more attractors' temporal exposomic data signal recurrence rates, determinism, mean diagonal length, maximum diagonal length, divergence, Shannon entropy in diagonal length, trend in recurrences, laminarity, trapping time, maximum vertical line length, Shannon entropy in vertical line lengths, mean recurrence time, Shannon entropy in recurrence times, and number of the most probable recurrences, or any combination thereof.

Embodiment 100. The method of embodiment 99, further comprising analyzing one or more features of the network of the one or more attractors to determine network connectivity, efficiency, feature importance, pathway importance, related graph-theory based metrics feature importance, pathway importance, or any combination thereof.

What is claimed:

1. A computer-implemented exposomics system, the system comprising:
one or more processors; and
memory addressable by the one or more processors, the memory storing:
(a) an exposome biochemical signature database (EDB), the EDB comprising, in electronic form, a corresponding plurality of exposomic features for each subject in a plurality of subjects that have or have not participated in space travel; and
(b) an intervention outcome database (IODB), the IODB comprising, in electronic form, information on intervention outcome information for at least one phase of at least one intervention of at least one subject of the plurality of subjects;
the memory further storing at least one program for execution by the one or more processors, the at least one program comprising:
(i) an association software module communicatively coupled to the EDB and the IODB, wherein the association software module is programmed to determine an association between the corresponding plurality of exposomic features and the intervention outcome information for at least one phase for the at least one subject, and
(ii) a recommendation software module communicatively coupled to the EDB and the IODB, the recommendation software module programmed to provide an intervention recommendation for the at least one subject based at least in part on the corresponding plurality of exposomic features of the at least one subject, the intervention outcome information for the at least one subject, and the association between the corresponding plurality of exposomic features, the clinical phenotype information, and the intervention outcome information for the at least one subject.

2. The computer-implemented exposomics system of claim 1, wherein the memory further stores a clinical database (CDB), the CDB comprising clinical phenotype information for the plurality of subjects, and wherein
the association software module is programmed to determine an association between the corresponding plurality of exposomic features, the clinical phenotype information, and the intervention outcome information for at least one subject; and
the recommendation software module communicatively is programmed to provide the intervention recommendation for the at least one subject based at least in part on the corresponding plurality of exposomic features of the at least one subject, the clinical phenotype information of the at least one subject, the intervention outcome information for the at least one subject, and the association between the corresponding plurality of exposomic features, the clinical phenotype information, and the intervention outcome information for the at least one subject.

3. The computer-implemented exposomics system of claim 1, wherein the EDB comprises a distinct corresponding plurality of exposomic features for each of at least 100, at least 1,000, or at least 10,000 subjects.

4. The computer-implemented exposomics system of claim 1, wherein the information on intervention outcome information for at least one phase of at least one intervention of at least one subject of the plurality of subjects comprises a classification of non-responder, adverse responder, or positive responder.

5. The computer-implemented exposomics system of claim 1, wherein the information on intervention outcome information for at least one phase of at least one intervention of at least one subject of the plurality of subjects comprises one or more inclusion criterion or exclusion criterion.

6. The computer-implemented exposomics system of claim 1, wherein the corresponding plurality of exposomic features for a subject in the plurality of subjects is obtained by assaying a biological sample of the subject.

7. The computer-implemented exposomics system of claim 6, wherein the biological sample comprises a tooth sample, a nail sample, a hair sample, or any combination thereof.

8. The computer-implemented exposomics system of claim 6, wherein the assaying comprises obtaining a mass spectrometry measurement, a laser ablation-inductively coupled plasma mass spectrometry measurement, a laser induced breakdown spectroscopy measurement, a Raman spectroscopy measurement, an immunohistochemistry measurement, a physiologic parameter, or any combination thereof.

9. The computer-implemented exposomics system of claim 8, wherein the assaying comprises obtaining one or more mass spectrometry measurement, wherein the one or more mass spectrometry measurements comprises measurements of one or more chemicals.

10. The computer-implemented exposomics system of claim 9, wherein the one or more chemicals comprise aluminum, arsenic, barium, bismuth, calcium, copper, iodide, lead, lithium, magnesium, manganese, phosphorus, sulfur, tin, strontium, zinc, or any combination thereof.

11. The computer-implemented exposomics system of claim 1, wherein the corresponding plurality of exposomic features of a corresponding subject in the plurality of subjects comprises one or more features of one or more dynamic temporal biochemical responses of the corresponding subject.

12. The computer-implemented exposomics system of claim 1, wherein the corresponding plurality of exposomic features for a corresponding subject in the plurality of subjects comprises one or more fluorescence images of one or more biological samples of the corresponding subject.

13. The computer-implemented exposomics system of claim 1, wherein the corresponding plurality of exposomic features for a corresponding subject in the plurality of subjects comprises one or more spatial maps of one or more Raman spectra of a biological sample of the corresponding subject.

14. The computer-implemented exposomics system of claim 1, wherein each corresponding plurality of exposomic features for each corresponding subject in the plurality of subjects is associated with an absence of space travel, or a presence of a space travel in the corresponding subject.

15. The computer-implemented exposomics system of claim 14, wherein the at least one program further comprises instructions for analyzing the corresponding plurality of exposomic features using a trained model to determine an association with space travel.

16. The computer-implemented exposomics system of claim 15, wherein the trained model is selected from the group consisting of: a neural network algorithm, a support vector machine algorithm, a decision tree algorithm, an unsupervised clustering algorithm, a supervised clustering algorithm, a regression algorithm, a gradient-boosting algorithm, and any combination thereof.

17. A method for selecting a subject for an intervention, the method comprising:
(a) providing a trained predictive model, wherein the trained predictive model is trained on one or more of clinical metadata, exposomic features, and corresponding intervention outcome information of a training cohort;
(b) detecting a biochemical signature using a biological sample obtained from the subject, thereby producing a plurality of prospective exposomic features;
(c) inputting the plurality of prospective exposomic features and clinical meta data of the subject into the trained predictive model thereby obtaining a predicted intervention outcome information of the subject; and
(d) selecting the subject for the intervention or excluding the subject from the intervention, based at least in part on the predicted intervention outcome information of the subject.

18. The method of claim 17, wherein the biochemical signature is obtained by assaying a biological sample of the subject.

19. The method of claim 18, wherein the biological sample comprises a tooth sample, a nail sample, a hair sample, or any combination thereof.

20. The method of claim 18, wherein the assaying comprises collecting data from laser ablation-inductively coupled plasma mass spectrometry measurements, laser induced breakdown spectroscopy measurements, Raman spectroscopy measurements, immunohistochemistry measurements, or any combination thereof.

21. The method of claim 18, wherein the assaying comprises collecting data from laser ablation-inductively coupled plasma mass spectrometry measurements and wherein the laser ablation-inductively coupled plasma mass spectrometry measurements comprise measurements of one or more element chemicals.

22. The method of claim 21, wherein the one or more element chemicals comprise aluminum, arsenic, barium, bismuth, calcium, copper, iodide, lead, lithium, magnesium, manganese, phosphorus, sulfur, tin, strontium, zinc, or any combination thereof.

23. The method of claim 17, wherein the biochemical signature comprises spatial maps of Raman spectra of the biological sample.

24. The method of claim 17, wherein the biochemical signature is associated with space travel.

25. The method of claim 17, wherein the intervention comprises a diet, pharmaceutical therapeutic, or any combination thereof.

26. The method of claim 17, wherein the trained predictive model is selected from the group consisting of: a neural network algorithm, a support vector machine algorithm, a decision tree algorithm, an unsupervised clustering algorithm, a supervised clustering algorithm, a regression algorithm, a gradient-boosting algorithm, and any combination thereof.

27. The method of claim 17, the method further comprising enrolling the subject into a study where the intervention will be provided when the subject is selected for the intervention.

28. The method of claim 17, further comprising evaluating the subject for a second intervention when the subject is excluded from the intervention.

29. A method of selecting an optimal treatment for subjects participating in space travel, comprising:
(a) detecting one or more biochemical signatures obtained from one or more biological samples from one or more subjects that have not participated in space travel, thereby producing one or more reference exposomic features;
(b) detecting features of one or more biochemical signature obtained from one or more biological samples from one or more subjects that have participated in space travel, thereby producing one or more pre-treatment exposomic features;
(c) administering a treatment to the one or more subjects that have participated in space travel;
(d) detecting features of one or more biochemical signatures obtained from one or more biological samples from the one or more subjects that have participated in space travel after a period of time has elapsed after receiving the treatment, thereby producing one or more post-treatment exposomic features;
(e) determining a difference between the one or more reference exposomic features of the one or more subjects that have not participated in space travel, the one or more pre-treatment exposomic features of the one or more subjects that have participated in space travel, and the one or more post-treatment exposomic features of the one or more subjects that have participated in space travel; and
(f) selecting one or more optimal treatments based at least in part on the determined difference between the one or more reference exposomic features, the one or more pre-treatment exposomic features, and the one or more post-treatment exposomic features, wherein the one or more optimal treatments are selected based on the determined differences satisfying a pre-determined criterion.

30. The method of claim 29, wherein an optimal treatment in the one or more optimal treatments comprises a pharmaceutical, nutraceutical, diet, food bar, or any combination thereof.

31. The method of claim 29, wherein the pre-determined criterion comprises a difference between the one or more pre-treatment exposomic features and the one or more post-treatment exposomic features to the one or more reference exposomic features.

32. The method of claim 29, wherein the period of time comprises at least about 1 hour, at least about 1 day, at least about 1 week, at least about 1 month, at least about 1 year, or any combination thereof.

33. The method of claim 29, wherein the difference comprises a change of at least 10% of the one or more post-treatment exposomic features toward the one or more reference exposomic features.

34. The method of claim 29, wherein the one or more pre-treatment exposomic features, the one or more post-treatment exposomic features, or any combination thereof is obtained by assaying a biological sample of a corresponding subject.

35. The method of claim 34, wherein the biological sample comprises a tooth sample, a nail sample, a hair sample, or any combination thereof.

36. The method of claim 34, wherein the assaying comprises obtaining laser ablation-inductively coupled plasma mass spectrometry, laser induced breakdown spectroscopy measurements, Raman spectroscopy measurements, immunohistochemistry measurements, or any combination thereof.

37. The method of claim 34, wherein the assaying comprises obtaining laser ablation-inductively coupled plasma mass spectrometry data and wherein the laser ablation-inductively coupled plasma mass spectrometry data comprises measurements of one or more element chemicals.

38. The method of claim 37, wherein the one or more element chemicals comprise aluminum, arsenic, barium, bismuth, calcium, copper, iodide, lead, lithium, magnesium, manganese, phosphorus, sulfur, tin, strontium, zinc, or any combination thereof.

39. The method of claim 29, wherein the one or more biochemical signatures comprise fluorescence images of the biological sample.

40. The method of claim 29, wherein the one or more biochemical signature comprises spatial maps of Raman spectra of the biological sample.

41. The method of claim 29, wherein the difference between the one or more reference exposomic features of the one or more subjects that have not participated in space, the one or more pre-treatment exposomic features of the one or more subjects that have participated in space travel, and the one or more post-treatment exposomic features of the one or more subjects that have participated in space travel are analyzed using a trained model.

42. The method of claim 41, wherein the trained model is selected from the group consisting of: a neural network algorithm, a support vector machine algorithm, a decision tree algorithm, an unsupervised clustering algorithm, a supervised clustering algorithm, a regression algorithm, a gradient-boosting algorithm, and any combination thereof.

43. A method for evaluating the effects of an intervention in a plurality of subjects, the method comprising:
sampling, for each respective subject in the plurality of subjects, each respective position in a plurality of positions along a reference line on a corresponding biological sample associated with chemical dynamics of the respective subject, thereby obtaining a corresponding plurality of chemical samples for the respective subject, each chemical sample in the plurality of chemical samples corresponding to a different position in the plurality of positions, and each position in the plurality of positions representing a different period of growth of the corresponding biological sample associated with chemical dynamics, wherein the plurality of positions comprises:
one or more positions representing a period of growth prior to the intervention,
one or more positions representing a period of growth during the intervention, and
one or more positions representing a period of growth after the intervention;
analyzing, for each respective subject in the plurality of subjects, the corresponding plurality of chemical samples for the respective subject with a mass spectrometer thereby obtaining a corresponding first dataset that includes a plurality of traces, each trace in the plurality of traces being a concentration of a corresponding chemicals, in a plurality of chemicals, over time collectively determined from the plurality of chemical samples,
generating, for each of one or more chemicals in the plurality of chemicals, a corresponding isotope data set comprising:
a set of pre-intervention features corresponding to the time period of biological sample growth prior to the intervention, the set of pre-intervention features comprising, for each respective subject in the plurality of subjects, one or more features derived from the concentration of the respective chemicals measured from the one or more positions representing a period of growth prior to the intervention,
a set of intervention features corresponding to the time period of biological sample growth during the intervention, the set of intervention features comprising, for each respective subject in the plurality of subjects, one or more features derived from the concentration of the respective chemicals measured from the one or more positions representing a period of growth during the intervention, and
a set of post-intervention features corresponding to the time period of biological sample growth after the intervention, the set of post-intervention features comprising, for each respective subject in the plurality of subjects, one or more features derived from the concentration of the respective chemicals measured from the one or more positions representing a period of growth after the intervention; and
evaluating changes in chemical dynamics in response to the intervention using, for each chemical in the one or more chemicals, the corresponding isotope data set.

44. The method of claim 43, wherein the evaluating comprises performing, for each of the plurality of chemicals, a probabilistic hypothesis test using (i) the set of pre-intervention features and (ii) one or both of the set of intervention features and the set of pos-intervention features.

45. A method for evaluating the effects of an intervention in a plurality of subjects, the method comprising:
sampling, for each respective subject in the plurality of subjects, each respective position in a plurality of positions along a reference line on a corresponding biological sample associated with chemical dynamics of the respective subject, thereby obtaining a corresponding plurality of chemical samples for the respective subject, each chemical sample in the plurality of chemical samples corresponding to a different position in the plurality of positions, and each position in the plurality of positions representing a different period of growth of the corresponding biological sample associated with chemical dynamics, wherein the plurality of positions comprises:
one or more positions representing a period of growth prior to the intervention,
one or more positions representing a period of growth during the intervention, and
one or more positions representing a period of growth after the intervention;
analyzing, for each respective subject in the plurality of subjects, the corresponding plurality of chemical samples for the respective subject with a mass spectrometer thereby obtaining a corresponding first dataset that includes a plurality of traces, each trace in the plurality of traces being a concentration of a corresponding chemicals, in a plurality of chemicals, over time collectively determined from the plurality of chemical samples,
generating, for a set of two or more chemicals in the plurality of chemicals, a respective aggregate isotope data set comprising:
a set of pre-intervention features corresponding to biological sample growth prior to the intervention, the set of pre-intervention features comprising values for one or more dimension reduction components formed from features derived from, for each respective subject in the plurality of subjects, concentrations of each of the two or more chemicals measured from the one or more positions representing a period of growth prior to the intervention,
a set of intervention features corresponding to biological sample growth during the intervention, the set of intervention features comprising values for one or more dimension reduction components formed from features derived from, for each respective subject in the plurality of subjects, concentrations of each of the two or more chemicals measured from the one or more positions representing a period of growth during the intervention, and
a set of post-intervention features corresponding to biological sample growth after the intervention, the set of post-intervention features comprising values for one or more dimension reduction components formed from features derived from, for each respective subject in the plurality of subjects, concentrations of each of the set of two or more chemicals measured from the one or more positions representing a period of growth after the intervention; and
evaluating changes in chemical dynamics in response to the intervention using the aggregate isotope data set.

46. The method of claim 45, wherein the evaluating comprises performing a probabilistic hypothesis test using (i) the set of pre-intervention features and (ii) one or both of the set of intervention features and the set of post-intervention features.

47. The method of claim 45 or 46, wherein, for each of the set of pre-intervention features, the set of intervention features, and the set of post-intervention features, the values for the dimension reduction components are determined from the features derived from the features of each of the two or more chemicals measured from a single respective subject in the plurality of subjects.

48. The method of claim 46 or 47, wherein, for each of the set of pre-intervention features, the set of intervention features, and the set of post-intervention features, the values for the dimension reduction components are determined from an aggregate of the features derived from the features of each of the set of two or more chemicals measured from a plurality of respective subjects in the plurality of subjects.

49. The method of any one of claims 45-48, wherein the one or more features derived from the concentration of the respective chemicals measured from the one or more positions representing a period of growth are the concentrations, normalized concentrations thereof, or related descriptive statistics or derived parameters thereof.

50. The method of any one of claims 44-49, wherein the one or more features derived from the concentration of the respective chemicals measured from the one or more positions representing a period of growth are selected from the group consisting of recurrence rates, determinism, mean diagonal length, maximum diagonal length, divergence, Shannon entropy in diagonal length, trend in recurrences, laminarity, trapping time, maximum vertical line length, Shannon entropy in vertical line lengths, mean recurrence time, Shannon entropy in recurrence times, and number of the most probable recurrences; these measures are derived through the application of recurrence quantification analysis parameters, cross-recurrence quantification analysis parameters, joint recurrence quantification analysis parameters, and/or multidimensional recurrence quantification analysis.

51. A method for evaluating the effects of an intervention in a plurality of subjects, the method comprising:
sampling, for each respective subject in the plurality of subjects, each respective position in a plurality of positions along a reference line on a corresponding biological sample associated with chemical dynamics of the respective subject, thereby obtaining a corresponding plurality of chemical samples for the respective subject, each chemical sample in the plurality of chemical samples corresponding to a different position in the plurality of positions, and each position in the plurality of positions representing a different period of growth of the corresponding biological sample associated with chemical dynamics, wherein the plurality of positions comprises:
one or more positions representing a period of growth prior to the intervention,
one or more positions representing a period of growth during the intervention, and
one or more positions representing a period of growth after the intervention;
analyzing, for each respective subject in the plurality of subjects, the corresponding plurality of chemical samples for the respective subject with a mass spectrometer thereby obtaining a corresponding first dataset that includes a plurality of traces, each trace in the plurality of traces being a concentration of a corresponding chemicals, in a plurality of chemicals, over time collectively determined from the plurality of chemical samples,
applying, for each respective subject in the plurality of subjects and for each of one or more chemicals in the plurality of chemicals, a distributed lag model or similar non-linear distribution model as a function of time relative to the intervention to the concentration of the respective chemicals measured from the plurality of positions, or a feature derived therefrom, to generate a corresponding contribution data set representing the contribution of the intervention to the concentration of the respective chemicals in the respective subject as a function of time; and
evaluating changes in chemical dynamics in response to the intervention using the corresponding contribution data set, for each of the one or more chemicals, from each respective subject in the plurality of subjects.

52. A method for evaluating the effects of an intervention in a plurality of subjects, the method comprising:
sampling, for each respective subject in the plurality of subjects, each respective position in a plurality of positions along a reference line on a corresponding biological sample associated with chemical dynamics of the respective subject, thereby obtaining a corresponding plurality of chemical samples for the respective subject, each chemical sample in the plurality of chemical samples corresponding to a different position in the plurality of positions, and each position in the plurality of positions representing a different period of growth of the corresponding biological sample associated with chemical dynamics, wherein the plurality of positions comprises:
one or more positions representing a period of growth prior to the intervention,
one or more positions representing a period of growth during the intervention, and
one or more positions representing a period of growth after the intervention;
analyzing, for each respective subject in the plurality of subjects, the corresponding plurality of chemical samples for the respective subject with a mass spectrometer thereby obtaining a corresponding first dataset that includes a plurality of traces, each trace in the plurality of traces being a concentration of a corresponding chemicals, in a plurality of chemicals, over time collectively determined from the plurality of chemical samples,
generating, for each respective subject in the plurality of subjects and for a set of two or more chemicals in the plurality of chemicals, a corresponding aggregate isotope data set comprising a set of features comprising values for one or more dimension reduction components formed from features derived from concentrations of each of the two or more chemicals measured across the different periods of growth for the respective subject;
applying, for each respective subject in the plurality of subjects, a distributed lag model or similar non-linear distribution model as a function of time relative to the intervention to the corresponding aggregate isotope data set to generate a corresponding contribution data set representing the contribution of the intervention to the concentration of the set of two or more chemicals in the respective subject as a function of time; and
evaluating changes in chemical dynamics in response to the intervention using the corresponding contribution data for each respective subject in the plurality of subjects.

53. The method of any one of claims 45-52, wherein the intervention is ingestion of a nutraceutical composition.

54. The method of claim 53, further comprising, in response to the evaluating changes in chemical dynamics, altering the composition of the nutraceutical composition to adjust the effects of the ingestion of the nutraceutical composition.

55. The method of claim 53, further comprising, in response to the evaluating changes in chemical dynamics, supplementing ingestion of the nutraceutical composition with ingestion of one or more dietary supplements.

56. The method of any one of claims 44-55, further comprising evaluating changes in the metabolism of one or more additional metabolites in response to the intervention.

57. The method of claim 56, wherein the one or additional metabolites are selected from the group consisting of a perfluoro compound, a paraben, a phthalate, a lipid, an amino acid, an amino acid derivative, and a peptide.

58. A method for outputting one or more quantitative metrics of one or more exposomic signatures of a first subject, comprising:
(a) receiving a biological sample from the subject;
(b) determining one or more exposomic signatures from the biological sample of the subject;
(c) calculating one or more features of the one or more exposomic signatures, wherein each feature of the one or more features comprises one or more quantitative metrics; and
(d) outputting the one or more quantitative metrics of the one or more features of the subject.

59. The method of claim 58, further comprising outputting a health outcome of the subject based at least in part on an association of normalized scores of the one or more features of the subject to normalized scores of a second set of one or more features of a second subject, wherein the subject and the second subject differ.

60. The method of claim 59, wherein the second set of the one or more features are stored in a database, wherein the database is a hosted on a local server, a cloud-based server, or a virtual machine.

61. The method of claim 59, wherein the health outcome comprises a diagnosis of a clinical subtype, non-clinical subgrouping related to physiology, anthropometric indicators, behavior indicators, socioeconomic indicators, body mass index, intelligence quotient, socio-economic status, or any combination thereof.

62. The method of claim 58, wherein the one or more features comprise a measurement of temporal dynamics of the one or more exposomic signatures.

63. The method of claim 62, wherein the measurement of the temporal dynamics comprises: determination of a linear slope, determination of a plurality of non-linear parameters describing curvature of the one or more exposomic signatures, determination of an abrupt change in intensity of the one or more exposomic signatures, determination of one or more changes in a baseline intensity of the one or more exposomic signatures, determination of a change of a frequency-domain representation of the one or more exposomic signatures, determination of a change of the power-spectral domain representation of the one or more exposomic signatures, determination of one or more recurrence quantification analysis parameters, determination of one or more cross-recurrence quantification analysis parameters, determination of one or more joint recurrence quantification analysis parameters, determination of one or more multidimensional recurrence quantification analysis parameters, estimation of a Lyapunov spectra, determination of a maximum Lyapunov exponent, or any combination thereof.

64. The method of claim 58, wherein the one or more features of the one or more exposomic signatures comprise phenotypic features, wherein the phenotypic features comprise: electrocardiogram (ECG), electroencephalography, magnetic resonance imaging (MRI), functional magnetic resonance imaging (fMRI), positron emission tomography (PET), genomic, epigenomic, transcriptomic, proteomic, metabolomic, or any combination thereof data.

65. The method of claim 58, wherein the one or more features are derived from one or more attractors.

66. The method of claim 58, wherein the one or more exposomic signatures are measured by mass spectrometry, laser ablation-inductively coupled plasma mass spectrometry, laser induced breakdown spectroscopy, Raman spectroscopy, immunohistochemistry fluorescence, or any combination thereof.

67. The method of claim 58, wherein the biological sample comprises hair, teeth, toenails, fingernails, physiologic parameters, or any combination thereof.

68. The method of claim 64, wherein the phenotypic features comprise molecular phenotypes.

69. The method of claim 68, wherein the molecular phenotypes are determined by unsupervised analysis, wherein unsupervised analysis comprises clustering, dimensionality-reduction, factor analysis, stacked autoencoding, or any combination thereof.

70. The method of claim 62, wherein the measurement of the temporal dynamics comprises determination of one or more the recurrence quantification analysis parameters, wherein the one or more the recurrence quantification analysis parameters comprises a recurrence rate, a determinism, a mean diagonal length, a maximum diagonal length, a divergence, a Shannon entropy in diagonal length, a trend in recurrence, a laminarity, a trapping time, a maximum vertical line length, a Shannon entropy in vertical line length, a mean recurrence time, a Shannon entropy in recurrence time, or a number of a most probable recurrence.

71. The method of claim 66, wherein the one or more exposomic signatures comprise measurements of one or more element chemical measured by the laser ablation-inductively coupled plasma mass spectrometry.

72. The method of claim 71, wherein the one or more element chemicals comprise aluminum, arsenic, barium, bismuth, calcium, copper, iodide, lead, lithium, magnesium, manganese, phosphorus, sulfur, tin, strontium, zinc, or any combination thereof.

73. The method of claim 58, wherein the one or more quantitative metrics comprise a measurement of temporal dynamics of the one or more exposomic signatures.

74. The method of claim 65, further comprising analyzing the one or more attractors by potential energy analysis thereby producing a potential energy data space.

75. The method of claim 58, wherein the one or more exposomic signatures of the subject comprises retrospective, prospective, or any combination thereof exposomic data.

76. The method of claim 65, further comprising analyzing a dynamic relationship between the one or more attractors' signal recurrence rates, determinism, mean diagonal length, maximum diagonal length, divergence, Shannon entropy in diagonal length, trend in recurrences, laminarity, trapping time, maximum vertical line length, Shannon entropy in vertical line lengths, mean recurrence time, Shannon entropy in recurrence times, number of the most probable recurrences, or any combination thereof.

77. The method of claim 76, wherein the dynamic relationship is determined by cross-convergent mapping (CCM).

78. The method of claim 58, further comprising reducing the one or more exposomic signatures to a reduced one or more exposomic signatures.

79. The method of claim 65, further comprising constructing a network of the one or more attractors based on similarity of the one or more attractors' temporal exposomic data signal recurrence rates, determinism, mean diagonal length, maximum diagonal length, divergence, Shannon entropy in diagonal length, trend in recurrences, laminarity, trapping time, maximum vertical line length, Shannon entropy in vertical line lengths, mean recurrence time, Shannon entropy in recurrence times, number of the most probable recurrences, or any combination thereof.

80. The method of claim 79, further comprising analyzing one or more features of the network of the one or more attractors to determine network connectivity, efficiency, feature importance, pathway importance, related graph-theory based metrics, or any combination thereof.

81. A method for outputting a prediction of phenotypic data of one or more subjects, comprising:
(a) receiving one or more biological samples and phenotypic data from a first set of subjects;
(b) determining a first set of exposomic signatures from one or more biological samples of the first set of subjects;
(c) calculating a first set of features of the first set of exposomic signatures;
(d) training a predictive model with the first set of features and the phenotypic data of the first set of subjects;
(e) receiving one or more biological samples from a second set of subjects different than the first set of subjects;
(f) determining a second set of exposomic signatures from the one or more biological samples of the second set of subjects;
(g) calculating a second set of features from the second set of exposomic signatures; and
(h) outputting the prediction of the second set of subjects' phenotypic data determined by inputting the second set of features into the trained predictive model.

82. The method of claim 81, wherein the first and second set of features comprise one or more quantitative metrics.

83. The method of claim 82, wherein the one or more quantitative metrics comprise a measurement of temporal dynamics of the one or more exposomic signatures.

84. The method of claim 83, wherein the measurement of the temporal dynamics comprises: determination of a linear slope, determination of one or more non-linear parameters describing curvature of the first and second set of exposomic signatures, determination of one or more abrupt changes in intensity of the first and second set of exposomic signatures, determination of one or more changes in baseline intensity of the first and second set of exposomic signatures, determination of one or more changes of the frequency-domain representation of the first and second set of exposomic signatures, determination of one or more changes of the power-spectral domain representation of the first and second set of exposomic signatures, determination of one or more recurrence quantification analysis parameters, determination of one or more cross-recurrence quantification analysis parameters, determination of one or more joint recurrence quantification analysis parameters, determination of one or more multidimensional recurrence quantification analysis parameters, estimation of a Lyapunov spectra or a maximum Lyapunov exponent, or any combination thereof.

85. The method of claim 81, wherein the first set of features of the first set of exposomic signatures comprise a first set of phenotypic features, and wherein the second set of features of the second set of exposomic signatures comprises a second set of phenotype features.

86. The method of claim 85, wherein the first set of phenotypic features or the second set of phenotypic comprises: electrocardiogram (ECG), electroencephalography, magnetic resonance imaging (MRI), functional magnetic resonance imaging (fMRI), positron emission tomography (PET), genomic, epigenomic, transcriptomic, proteomic, metabolomic, or any combination thereof data.

87. The method of claim 81, wherein the first and second set of features are represented as or derived from one or more attractors.

88. The method of claim 87, wherein the one or more attractors are a limit cycle attractor, bistable attractor, or any combination thereof.

89. The method of claim 81, wherein the first and second set of exposomic signatures are measured by mass spectrometry, laser ablation-inductively coupled plasma mass spectrometry, laser induced breakdown spectroscopy, Raman spectroscopy, immunohistochemistry fluorescence, or any combination thereof.

90. The method of claim 81, wherein the one or more biological samples of the first and second subjects comprise hair, teeth, toenails, fingernails, physiologic parameters, or any combination thereof.

91. The method of claim 85, wherein the first set of phenotypic features and the second set of phenotypic features each comprise a plurality of molecular phenotypes.

92. The method of claim 91, wherein the molecular phenotypes are determined by unsupervised analysis, wherein unsupervised analysis comprises clustering, dimensionality-reduction, factor analysis, stacked autoencoding, or any combination thereof.

93. The method of claim 83 wherein the measurement of the temporal dynamics comprises a determination of a one or more recurrence quantification analysis parameters, wherein the one or more recurrence quantification analysis parameters comprise one or more recurrence rates, a determinism, a mean diagonal length, a maximum diagonal length, a divergence, a Shannon entropy in diagonal length, a trend in recurrences, a laminarity, a trapping time, a maximum vertical line length, a Shannon entropy in vertical line lengths, a mean recurrence time, a Shannon entropy in recurrence times, a number of the most probable recurrences, or any combination thereof.

94. The method of claim 87, further comprising analyzing the one or more attractors by potential energy analysis thereby producing a potential energy data space.

95. The method of claim 81, wherein the first and second set of exposomic signatures comprises retrospective, prospective, or any combination thereof dynamic exposomic data.

96. The method of claim 87, further comprising analyzing a dynamic relationship between the one or more attractors' recurrence rates, determinism, mean diagonal length, maximum diagonal length, divergence, Shannon entropy in diagonal length, trend in recurrences, laminarity, trapping time, maximum vertical line length, Shannon entropy in vertical line lengths, mean recurrence time, Shannon entropy in recurrence times, and number of the most probable recurrences, or any combination.

97. The method of claim 96, wherein the dynamic relationship is determined by cross-convergent mapping (CCM).

98. The method of claim 87, further comprising constructing a network of the one or more attractors based on similarity of the one or more attractors' temporal exposomic data signal recurrence rates, determinism, mean diagonal length, maximum diagonal length, divergence, Shannon entropy in diagonal length, trend in recurrences, laminarity, trapping time, maximum vertical line length, Shannon entropy in vertical line lengths, mean recurrence time, Shannon entropy in recurrence times, and number of the most probable recurrences, or any combination thereof.

99. The method of claim 98, further comprising analyzing one or more features of the network of the one or more attractors to determine network connectivity, efficiency, feature importance, pathway importance, related graph-theory based metrics feature importance, pathway importance, or any combination thereof.

100. The method of claim 17, wherein the biochemical signature comprises fluorescence images of the biological sample.

101. The computer-implemented exposomics system of claim 16, wherein the trained model is a regressor or a classifier.

102. The computer-implemented exposomics system of claim 16, wherein the trained model comprises one or more regression tasks, one or more classification tasks, or a combination of both one or more regression tasks and one or more classification tasks.

103. The method of claim 17, wherein the trained predictive model is a regressor

104. The method of claim 17, wherein the trained predictive model is a classifier.

105. The method of claim 17, wherein the trained predictive model comprises one or more regression tasks, one or more classification tasks, or a combination of both one or more regression tasks and one or more classification tasks.

106. The method of claim 42, wherein the trained model is a regressor or a classifier.

107. The method of claim 42, wherein the trained model comprises one or more regression tasks, one or more classification tasks, or a combination of both one or more regression tasks and one or more classification tasks.

108. A method for outputting one or more subjects' response to participating in real or simulated space travel, comprising:
(a) receiving one or more biological samples of one or more subjects participating in space travel;
(b) determining one or more exposomic signatures of the one or more biological samples;
(c) calculating exposomic features of the one or more biological samples' one or more exposomic signatures; and
(d) outputting the one or more subjects' response to participating in space travel as an output of a trained predictive model provided the exposomic features of the one or more biological samples as an input.

109. The method of claim 108, wherein the trained predictive model is configured to determine a likelihood that the exposomic features are associated with subjects participating in real or simulated space travel.

110. The method of claim 109, wherein the trained predictive model is configured to determine if subjects' metabolism is at baseline conditions not associated with space travel.

111. The method of claim 108, wherein each feature of the exposomic features comprises one or more quantitative metrics.

112. The method of claim 108, wherein the one or more biological samples of the subjects participating in the space travel is collected prior to the space travel, during the space travel, after the space travel, or any combination thereof periods of the space travel.

113. The method of claim 108, wherein space travel comprises traveling beyond the Karman line at an altitude of at least 100 kilometers (km) above the Earth's surface.

114. The method of claim 108, wherein the biological sample comprises hair, teeth, toenails, fingernails, physiologic parameters, or any combination thereof.

115. The method of claim 108, wherein the exposomic features comprise a measurement of temporal dynamics of the one or more exposomic signatures.

116. The method of claim 115, wherein the measurement of the temporal dynamics comprises: determination of a linear slope, determination of a plurality of non-linear parameters describing curvature of the one or more exposomic signatures, determination of an abrupt change in intensity of the one or more exposomic signatures, determination of one or more changes in a baseline intensity of the one or more exposomic signatures, determination of a change of a frequency-domain representation of the one or more exposomic signatures, determination of a change of the power-spectral domain representation of the one or more exposomic signatures, determination of one or more recurrence quantification analysis parameters, determination of one or more cross-recurrence quantification analysis parameters, determination of one or more joint recurrence quantification analysis parameters, determination of one or more multidimensional recurrence quantification analysis parameters, estimation of a Lyapunov spectra, determination of a maximum Lyapunov exponent, or any combination thereof.

117. The method of claim 116, wherein the measurement of the temporal dynamics comprises determination of one or more recurrence quantification analysis parameters, wherein the one or more recurrence quantification analysis parameters comprises a recurrence rate, a determinism, a mean diagonal length, a maximum diagonal length, a divergence, a Shannon entropy in diagonal length, a trend in recurrence, a laminarity, a trapping time, a maximum vertical line length, a Shannon entropy in vertical line length, a mean recurrence time, a Shannon entropy in recurrence time, or a number of a most probable recurrence.

118. The method of claim 117, wherein the one or more exposomic signatures are measured by mass spectrometry, laser ablation-inductively coupled plasma-mass spectrometry, laser induced breakdown spectroscopy, Raman spectroscopy, immunohistochemistry fluorescence, or any combination thereof.

119. The method of claim 108, wherein the exposomic features are derived from one or more attractors.

120. The method of claim 119, further comprising analyzing a dynamic relationship between the one or more attractors' signal recurrence rates, determinism, mean diagonal length, maximum diagonal length, divergence, Shannon entropy in diagonal length, trend in recurrences, laminarity, trapping time, maximum vertical line length, Shannon entropy in vertical line lengths, mean recurrence time, Shannon entropy in recurrence times, number of the most probable recurrences, or any combination thereof.

121. The method of claim 108, wherein the one or more subjects are a human, non-human mammal, vertebrate, or any combination thereof.

122. The method of claim 108, wherein the trained predictive model comprises a classification algorithm.

123. The method of claim 122, wherein the classification algorithm is configured to identify the one or more exposomic signatures of the one or more subjects associated with participating in space travel.

124. The method of claim 122, wherein the classification algorithm is trained with the one or more subjects' one or more biological samples exposomic features and corresponding responses, wherein the one or more subjects participated in space travel.

125. The method of claim 122, wherein the classification algorithm is configured to determine if the one or more subjects have a metabolic signature associated with space travel.

126. The method of claim 108, wherein the response comprises a state of the subject's metabolism, wherein the subject's metabolism comprises a healthy state or a state consistent with a subject undergoing space-travel.

127. A method for training a predictive model to determine a clinical response of subjects to an intervention when the subject is participating in space travel, comprising:
(a) receiving one or more biological samples and clinical responses of a first set of subjects, wherein the first set of subjects are exposed to space travel and are provided an intervention, and a second set of subjects, wherein the second set of subjects are exposed to space travel and do not receive the intervention;
(b) determining one or more exposomic signatures of the one or more biological samples of the first and second set of subjects;
(c) calculating a first and second set of exposomic features of the first and second set of subjects' one or more exposomic signatures, wherein each feature of the first and second set of exposomic features comprises one or more quantitative metrics; and
(a) outputting a trained predictive model configured to determine a clinical response of a subject to an intervention, wherein the subject is participating in space travel, and wherein the trained predictive model is trained on the first and second set of subjects' one or more exposomic features' one or more quantitative metrics and the corresponding clinical responses.

128. The method of claim 127, wherein the intervention prevents negative side effects of space travel.

129. The method of claim 127, wherein the intervention is provided to the first set of subjects prior to, during, after, or any combination thereof time point of space travel the subjects have been exposed to.

130. The method of claim 127, further comprising determining an influence of space travel on the first set of subjects exposed to the intervention at least in part by comparing the first and second set of exposomic features' one or more quantitative metrics.

131. The method of claim 127, wherein the intervention comprises a diet, pharmaceutical therapeutic, food bar, or any combination thereof.

132. The method of claim 131, wherein the diet comprises iodine supplementation.

133. The method of claim 127, wherein the first or second set of exposomic features comprise a measurement of temporal dynamics of the one or more exposomic signatures.

134. The method of claim 133, wherein the measurement of the temporal dynamics comprises: determination of a linear slope, determination of a plurality of non-linear parameters describing curvature of the one or more exposomic signatures, determination of an abrupt change in intensity of the one or more exposomic signatures, determination of one or more changes in a baseline intensity of the one or more exposomic signatures, determination of a change of a frequency-domain representation of the one or more exposomic signatures, determination of a change of the power-spectral domain representation of the one or more exposomic signatures, determination of one or more recurrence quantification analysis parameters, determination of one or more cross-recurrence quantification analysis parameters, determination of one or more joint recurrence quantification analysis parameters, determination of one or more multidimensional recurrence quantification analysis parameters, estimation of a Lyapunov spectra, determination of a maximum Lyapunov exponent, or any combination thereof.

135. The method of claim 133, wherein the measurement of the temporal dynamics comprises determination of one or more the recurrence quantification analysis parameters, wherein the one or more the recurrence quantification analysis parameters comprises a recurrence rate, a determinism, a mean diagonal length, a maximum diagonal length, a divergence, a Shannon entropy in diagonal length, a trend in recurrence, a laminarity, a trapping time, a maximum vertical line length, a Shannon entropy in vertical line length, a mean recurrence time, a Shannon entropy in recurrence time, or a number of a most probable recurrence.

136. The method of claim 127, wherein the one or more exposomic signatures are measured by mass spectrometry, laser ablation-inductively coupled plasma-mass spectrometry, laser induced breakdown spectroscopy, Raman spectroscopy, immunohistochemistry fluorescence, or any combination thereof.

137. The method of claim 127, wherein the one or more biological samples comprise hair, teeth, toenails, fingernails, physiologic parameters, or any combination thereof.

138. The method of claim 127, wherein the first or second set of exposomic features are derived from one or more attractors.

139. The method of claim 138, further comprising analyzing a dynamic relationship between the one or more attractors' signal recurrence rates, determinism, mean diagonal length, maximum diagonal length, divergence, Shannon entropy in diagonal length, trend in recurrences, laminarity, trapping time, maximum vertical line length, Shannon entropy in vertical line lengths, mean recurrence time, Shannon entropy in recurrence times, number of the most probable recurrences, or any combination thereof.

140. The method of claim 127, wherein the first or second set of subjects are human, non-human mammals, or any combination thereof.

141. A method for generating a predictive model configured to output one or more subjects' health state in response to a gravitational force in addition to the earth's gravitational force, comprising:
(a) receiving a first one or more subjects' one or more biological samples and corresponding health states, wherein the first one or more subjects' have received an exposure to a gravitational force in addition to the earth's gravitational force;
(b) determining one or more exposomic signatures of the first one or more subjects' one or more biological samples;
(c) calculating exposomic features of the one or more biological samples' one or more exposomic signatures; and
(d) generating a trained predictive mode configured to output a second one or more subjects' health state in response to a gravitational force in addition to the earth's gravitational force, wherein the trained predictive model is trained with the first one or more subjects' exposomic features of the one or more exposomic signatures and corresponding health states.

142. The method of claim 141, wherein the gravitational force comprises at least 1 G, at least 2 G, at least 3G, or at least 4G.

143. The method of claim 141, wherein the trained predictive model is configured to determine a likelihood that the exposomic features are associated with metabolic signatures of the first or second one or more subjects exposed to a gravitational force in addition to the earth's gravitational force.

144. The method of claim 141, wherein the trained predictive model indicates if the first or second one or more subjects' metabolism is at baseline conditions not associated with an exposure to a gravitational force in addition to the earth's gravitational force.

145. The method of claim 141, wherein each feature of the exposomic features comprises one or more quantitative metrics.

146. The method of claim 141, wherein the one or more biological samples comprise hair, teeth, toenails, fingernails, physiologic parameters, or any combination thereof.

147. The method of claim 141, wherein the exposomic features comprise a measurement of temporal dynamics of the one or more exposomic signatures.

148. The method of claim 147, wherein the measurement of the temporal dynamics comprises: determination of a linear slope, determination of a plurality of non-linear parameters describing curvature of the one or more exposomic signatures, determination of an abrupt change in intensity of the one or more exposomic signatures, determination of one or more changes in a baseline intensity of the one or more exposomic signatures, determination of a change of a frequency-domain representation of the one or more exposomic signatures, determination of a change of the power-spectral domain representation of the one or more exposomic signatures, determination of one or more recurrence quantification analysis parameters, determination of one or more cross-recurrence quantification analysis parameters, determination of one or more joint recurrence quantification analysis parameters, determination of one or more multidimensional recurrence quantification analysis parameters, estimation of a Lyapunov spectra, determination of a maximum Lyapunov exponent, or any combination thereof.

149. The method of claim 147, wherein the measurement of the temporal dynamics comprises determination of one or more recurrence quantification analysis parameters, wherein the one or more recurrence quantification analysis parameters comprises a recurrence rate, a determinism, a mean diagonal length, a maximum diagonal length, a divergence, a Shannon entropy in diagonal length, a trend in recurrence, a laminarity, a trapping time, a maximum vertical line length, a Shannon entropy in vertical line length, a mean recurrence time, a Shannon entropy in recurrence time, or a number of a most probable recurrence.

150. The method of claim 141, wherein the one or more exposomic signatures are measured by mass spectrometry, laser ablation-inductively coupled plasma-mass spectrometry, laser induced breakdown spectroscopy, Raman spectroscopy, immunohistochemistry fluorescence, or any combination thereof.

151. The method of claim 141, wherein the exposomic features are derived from one or more attractors.

152. The method of claim 151, further comprising analyzing a dynamic relationship between the one or more attractors' signal recurrence rates, determinism, mean diagonal length, maximum diagonal length, divergence, Shannon entropy in diagonal length, trend in recurrences, laminarity, trapping time, maximum vertical line length, Shannon entropy in vertical line lengths, mean recurrence time, Shannon entropy in recurrence times, number of the most probable recurrences, or any combination thereof.

153. The method of claim 141, wherein the first and second one or more subjects are human, non-human mammal, vertebrate, or any combination thereof.

154. The method of claim 141, wherein the trained predictive model comprises a classification algorithm.

155. The method of claim 154, wherein the classification algorithm is configured to identify the one or more exposomic signatures of the first or second one or more subject associated with exposure to the gravitational force in addition to the earth's gravitational force.

156. The method of claim 154, wherein the classification algorithm is trained with the first one or more subjects' one or more biological samples' exposomic features and corresponding health states, wherein the first one or more one or more subjects were exposed to the gravitational force in addition to the earth's gravitational force.

157. The method of claim 154, wherein the classification algorithm is configured to determine if the first or second one or more subjects' have metabolic signatures associated with exposure to the gravitational force in addition to the earth's gravitational force.

158. The method of claim 154, wherein the health state comprises a state of the first or second one or more subjects' metabolism, wherein the first or second one or more subjects' metabolism comprises a healthy state or a state consistent with a subject exposed to the gravitational force in addition to the earth's gravitational force.

159. A method for using a predictive model to output a health outcome of one or more subjects prior to exposure of a gravitational force, comprising:
(a) receiving one or more subjects' one or more biological samples;
(b) determining one or more exposomic signatures of the one or more subjects' one or more biological samples;
(c) calculating exposomic features of the one or more biological samples' one or more exposomic signatures; and
(d) outputting a health outcome of the one or more subjects prior to exposure of a gravitational force in addition to the gravitational force of the earth by providing the one or more subjects' exposomic features as an input to a trained predictive model.

160. The method of claim 159, wherein the gravitational force comprises at least 1 G, at least 2 G, at least 3G, or at least 4G.

161. The method of claim 159, wherein the trained predictive model is configured to determine a likelihood that the exposomic features are associated with subjects' exposure to the gravitational force.

162. The method of claim 161, wherein a probabilistic threshold determined by the trained predictive model indicates that one or more subjects' metabolism is at baseline conditions not associated with the exposure to the gravitational force in addition to the earth's gravitational force.

163. The method of claim 159, wherein each feature of the exposomic features comprises one or more quantitative metrics.

164. The method of claim 159, wherein the one or more biological samples comprise hair, teeth, toenails, fingernails, physiologic parameters, or any combination thereof.

165. The method of claim 159, wherein the exposomic features comprise a measurement of temporal dynamics of the one or more exposomic signatures.

166. The method of claim 165, wherein the measurement of the temporal dynamics comprises: determination of a linear slope, determination of a plurality of non-linear parameters describing curvature of the one or more exposomic signatures, determination of an abrupt change in intensity of the one or more exposomic signatures, determination of one or more changes in a baseline intensity of the one or more exposomic signatures, determination of a change of a frequency-domain representation of the one or more exposomic signatures, determination of a change of the power-spectral domain representation of the one or more exposomic signatures, determination of one or more recurrence quantification analysis parameters, determination of one or more cross-recurrence quantification analysis parameters, determination of one or more joint recurrence quantification analysis parameters, determination of one or more multidimensional recurrence quantification analysis parameters, estimation of a Lyapunov spectra, determination of a maximum Lyapunov exponent, or any combination thereof.

167. The method of claim 165, wherein the measurement of the temporal dynamics comprises determination of one or more recurrence quantification analysis parameters, wherein the one or more recurrence quantification analysis parameters comprises a recurrence rate, a determinism, a mean diagonal length, a maximum diagonal length, a divergence, a Shannon entropy in diagonal length, a trend in recurrence, a laminarity, a trapping time, a maximum vertical line length, a Shannon entropy in vertical line length, a mean recurrence time, a Shannon entropy in recurrence time, or a number of a most probable recurrence.

168. The method of claim 159, wherein the one or more exposomic signatures are measured by mass spectrometry, laser ablation-inductively coupled plasma-mass spectrometry, laser induced breakdown spectroscopy, Raman spectroscopy, immunohistochemistry fluorescence, or any combination thereof.

169. The method of claim 159, wherein the exposomic features are derived from one or more attractors.

170. The method of claim 169, further comprising analyzing a dynamic relationship between the one or more attractors' signal recurrence rates, determinism, mean diagonal length, maximum diagonal length, divergence, Shannon entropy in diagonal length, trend in recurrences, laminarity, trapping time, maximum vertical line length, Shannon entropy in vertical line lengths, mean recurrence time, Shannon entropy in recurrence times, number of the most probable recurrences, or any combination thereof.

171. The method of claim 159, wherein the one or more subjects comprise a human, non-human mammal, vertebrate, or any combination thereof.

172. The method of claim 159, wherein the trained predictive model comprises a classification algorithm.

173. The method of claim 172, wherein the classification algorithm is configured to identify the one or more exposomic signatures of the subject associated with an exposure to the gravitational force in addition to the earth's gravitational force.

174. The method of claim 172, wherein the classification algorithm is trained with one or more subjects' one or more biological samples' exposomic features and corresponding responses, wherein the one or more subjects are exposed to a gravitational force in addition to the earth's gravitational force.

175. The method of claim 172, wherein the classification algorithm is configured to determine if the one or more subjects' have metabolic signatures associated with exposure to a gravitational force in addition to the earth's gravitational force.

176. A method for administering a treatment to a subject participating in space travel, comprising:
(a) receiving one or more biological samples of a subject prior to participating in space travel;
(b) determining one or more exposomic signatures of the one or more biological samples of the subject;
(c) calculating exposomic features of the subject's one or more exposomic signatures, wherein each feature of the exposomic features comprises one or more quantitative metrics; and
(d) administering a treatment to the subject participating in space travel at a time point, wherein the treatment and the time point of administering the treatment is determined as an output of a trained predictive model when the trained predictive model is provided the exposomic features of the subject as an input.

177. The method of claim 176, wherein the trained predictive model is configured to output an efficacy of the treatment.

178. The method of claim 176, wherein the trained predictive model is configured to output a measure of whether or not the treatment is efficacious.

179. The method of claim 176, wherein the time point comprises a time prior to, during, after, or any combination thereof time of the subject participating in the space travel.

180. The method of claim 176, wherein, the treatment is provided to mitigate negative side-effects of participating in space travel.

181. The method of claim 177, wherein the efficacy of the treatment is determined by the duration of time after the subject's participation in space travel after which the subject's exposomic features comprise baseline exposomic features of a subject that has not participated in space travel.

182. The method of claim 176, wherein the trained predictive model comprises a classification algorithm.

183. The method of claim 176, wherein space travel comprises traveling beyond the Karman line at an altitude of at least 100 kilometers (km) above the Earth's surface.

184. The method of claim 176, wherein the one or more biological samples comprise hair, teeth, toenails, fingernails, physiologic parameters, or any combination thereof.

185. The method of claim 176, wherein the exposomic features comprise a measurement of temporal dynamics of the one or more exposomic signatures.

186. The method of claim 185, wherein the measurement of the temporal dynamics comprises: determination of a linear slope, determination of a plurality of non-linear parameters describing curvature of the one or more exposomic signatures, determination of an abrupt change in intensity of the one or more exposomic signatures, determination of one or more changes in a baseline intensity of the one or more exposomic signatures, determination of a change of a frequency-domain representation of the one or more exposomic signatures, determination of a change of the power-spectral domain representation of the one or more exposomic signatures, determination of one or more recurrence quantification analysis parameters, determination of one or more cross-recurrence quantification analysis parameters, determination of one or more joint recurrence quantification analysis parameters, determination of one or more multidimensional recurrence quantification analysis parameters, estimation of a Lyapunov spectra, determination of a maximum Lyapunov exponent, or any combination thereof.

187. The method of claim 185, wherein the measurement of the temporal dynamics comprises determination of one or more recurrence quantification analysis parameters, wherein the one or more recurrence quantification analysis parameters comprises a recurrence rate, a determinism, a mean diagonal length, a maximum diagonal length, a divergence, a Shannon entropy in diagonal length, a trend in recurrence, a laminarity, a trapping time, a maximum vertical line length, a Shannon entropy in vertical line length, a mean recurrence time, a Shannon entropy in recurrence time, or a number of a most probable recurrence.

188. The method of claim 176, wherein the one or more exposomic signatures are measured by mass spectrometry, laser ablation-inductively coupled plasma-mass spectrometry, laser induced breakdown spectroscopy, Raman spectroscopy, immunohistochemistry fluorescence, or any combination thereof.

189. The method of claim 176, wherein the exposomic features are derived from one or more attractors.

190. The method of claim 189, further comprising analyzing a dynamic relationship between the one or more attractors' signal recurrence rates, determinism, mean diagonal length, maximum diagonal length, divergence, Shannon entropy in diagonal length, trend in recurrences, laminarity, trapping time, maximum vertical line length, Shannon entropy in vertical line lengths, mean recurrence time, Shannon entropy in recurrence times, number of the most probable recurrences, or any combination thereof.

191. The method of claim 176, wherein the subject is a human, non-human mammal, or any combination thereof.

192. The method of claim 176, wherein the trained predictive model comprises a classification algorithm.

193. The method of claim 192, wherein the classification algorithm is trained with one or more subjects' one or more biological samples' exposomic features, treatment administered, time point of administering the treatment, and corresponding clinical response, wherein the one or more subjects participated in space travel.

194. The method of claim 192, wherein the classification algorithm is configured to determine if one or more subjects have a dysregulated metabolism induced by the space travel.

195. A method for evaluating one or more subjects' response to an intervention in the presence or lack thereof gravity, comprising:
(a) sampling each respective position in a plurality of positions along a reference line of a first set of one or more biological samples of a first set of subjects and a second set of one or more biological samples of a second set of subjects, thereby obtaining a plurality of ion samples, each ion sample in the plurality of ion samples corresponding to a different position in the plurality of positions, and each position in the plurality of positions representing a different period of growth of the first and second set of one or more biological samples, wherein the first set of subjects are subjected to space travel and the second set of subjects are not subjected to space travel, and wherein the first and second set of subjects are administered an intervention;
(b) analyzing each ion sample in the plurality of ion samples with a mass spectrometer thereby obtaining a first dataset that includes a plurality of traces, each trace in the plurality of traces being a concentration of a corresponding elemental isotope, in a plurality of elemental isotopes, over time collectively determined from the plurality of ion samples;
(c) deriving a second dataset from the plurality of traces that includes a set of features, each respective feature in the set of features being determined by a variation of a single isotope or a combination of isotopes in the plurality of traces; and
(d) inputting the set of features into a trained classifier thereby obtaining a probability from the trained classifier of the first and second set of one or more subjects' which predicts the one or more subjects' response to the space travel.

196. The method of claim 195, wherein the probability indicates that a biological sample of the one or more biological samples has been dysregulated as a result of the space travel.

197. The method of claim 196, wherein an efficacy of the intervention is measured by the probability of dysregulation.

198. The method of claim 195, wherein the intervention comprises a diet, pharmaceutical therapeutic, or any combination thereof.

199. The method of claim 198, wherein the diet comprises iodine supplementation.

200. The method of claim 195, wherein the intervention ameliorates radiation exposure.

201. The method of claim 195, wherein the first or second set of subjects comprise a human, non-human mammal, or any combination thereof.

202. The method of claim 201, wherein the non-human mammal comprises mice.

203. The method of claim 195, wherein the first or second set of one or more biological samples comprise hair, teeth, toenails, fingernails, physiologic parameters, or any combination thereof.

204. The method of claim 195, comprising treating the first or second set of one or more biological samples with a solvent or a surfactant prior to the sampling.

205. The method of claim 195, comprising irradiating the first or second set of one or more biological samples with a low powered laser to remove debris from the first or second set of one or more biological samples prior to the sampling.

206. The method of claim 195, wherein the sampling including irradiating, with a laser, the first or second set of one or more biological samples of the first or second set of subjects thereby extracting a plurality of particles from the first or second set of one or more biological samples and ionizing the plurality of particles with an inductively coupled plasma mass spectrometer, thereby obtaining the plurality of ion samples.

207. The method of claim 195, wherein the first or second set of subjects' responses comprise adverse response, positive response, or neutral response.

208. The method of claim 195, wherein the trained classifier is configured to identify a set of features of a subject associated with participating in space travel.

209. The method of claim 195, wherein the trained classifier is trained with the first set of subjects' one or more biological samples' set of features and corresponding responses.

210. The method of claim 195, wherein the trained classifier is configured to determine if a subject comprises a dysregulated metabolism induced by the space travel.

211. A method for outputting a subject's response to an intervention following space travel, comprising:
(a) exposing a subject's one or more biological samples to an optical signal comprising a plurality of Raman wavelengths, wherein the subject is subjected to space travel;
(b) acquiring a plurality of Raman spectra from the one or more biological samples;
(c) processing the plurality of Raman spectra to generate a plurality of Raman features; and
(d) outputting a probability of a subject's response to an intervention when subjected to space travel as a result of providing a trained classifier an input of the plurality of Raman features.

212. The method of claim 211, wherein the probability indicates that the subject has been dysregulated as a result of the space travel.

213. The method of claim 211, wherein an efficacy of the intervention is measured by the probability of dysregulation.

214. The method of claim 211, wherein the one or more biological samples comprise hair, teeth, toenails, fingernails, physiologic parameters, or any combination thereof.

215. The method of claim 211, wherein the intervention comprises a diet, pharmaceutical therapeutic, or any combination thereof.

216. The method of claim 215, wherein the diet comprises iodine supplementation.

217. The method of claim 215, wherein the intervention ameliorates radiation exposure.

218. The method of claim 215, wherein the subject comprises a human, non-human mammal, or any combination thereof.

219. The method of claim 218, wherein the non-human mammal comprises mice.

220. The method of claim 211, wherein the plurality of Raman spectra comprises from about 200 to about 3700 wave numbers.

221. The method of any one of claim 211, wherein acquiring comprises using a Raman spectroscopy microscope.

222. The method of claim 211, wherein the plurality of Raman features comprise one or more traces derived from a Raman spatial map.

223. The method of claim 222, wherein the one or more traces are reduced using a dimensionality-reduction algorithm, wherein the dimensionality-reduction algorithm comprises principal component analyses, independent component analysis, or any combination thereof algorithm.

224. The method of claim 223, wherein a measurement of temporal dynamics is derived from the one or more traces, wherein the temporal dynamics comprise: determination of a linear slope, determination of a plurality of non-linear parameters describing curvature of the one or more exposomic signatures, determination of an abrupt change in intensity of the one or more exposomic signatures, determination of one or more changes in a baseline intensity of the one or more exposomic signatures, determination of a change of a frequency-domain representation of the one or more exposomic signatures, determination of a change of the power-spectral domain representation of the one or more exposomic signatures, determination of one or more recurrence quantification analysis parameters, determination of one or more cross-recurrence quantification analysis parameters, determination of one or more joint recurrence quantification analysis parameters, determination of one or more multidimensional recurrence quantification analysis parameters, estimation of a Lyapunov spectra, determination of a maximum Lyapunov exponent, or any combination thereof.

225. The method of claim 224, wherein the measurement of the temporal dynamics comprises determination of one or more the recurrence quantification analysis parameters, wherein the one or more the recurrence quantification analysis parameters comprises a recurrence rate, a determinism, a mean diagonal length, a maximum diagonal length, a divergence, a Shannon entropy in diagonal length, a trend in recurrence, a laminarity, a trapping time, a maximum vertical line length, a Shannon entropy in vertical line length, a mean recurrence time, a Shannon entropy in recurrence time, or a number of a most probable recurrence.

226. The method of claim 211, wherein the trained classifier comprises a gradient-boosted ensemble model.

227. The method of claim 211, wherein the trained classifier is configured to process one or more features selected from the group consisting of laminarity, entropy, trapping time (TT), mean diagonal length (MDL), recurrence time (RT), Vmax, determinism, Lmax, and any combination thereof.

228. The method of claim 211, wherein the trained classifier is configured to process two or more features selected from the group consisting of laminarity, entropy, trapping time (TT), mean diagonal length (MDL), recurrence time (RT), Vmax, determinism, Lmax, and any combination thereof.

229. The method of claim 211, wherein the trained classifier is trained to identify Raman signatures associated with space travel.

230. The method of claim 211, wherein the output probability of the trained classifier indicates that the subject is exhibiting temporal dynamics associated with space travel.

231. The method of claim 211, wherein the trained classifier is trained with a population of human or non-human animals' plurality of Raman features, wherein the population of human or non-human animals have experienced space travel.

232. The method of claim 211, wherein an efficacy of the intervention is measured by a change of the subject's temporal dynamics towards a baseline temporal dynamics of the subject prior to space travel.

233. A method for evaluating a subject's response to an intervention to reduce the biological effects of space travel, comprising:
(a) staining a first set of one or more biological samples of a first set of subject and a second set of one or more biological samples of a second set of subjects to produce a stained first and second set of one or more biological samples, wherein the first set of subject are subjected to space travel and the second set of subjects are not subjected to space travel, and wherein the first and second set of subjects are administered an intervention;
(b) analyzing a fluorescence intensity spatially across the stained first and second set of one or more biological samples; and
(c) calculating a set of features of the first and second set of subjects' spatial fluorescent intensity across the stained first and second set of one or more biological samples, wherein each feature of the first and second set of subjects' exposomic features comprises one or more metrics quantifying temporal dynamics in the fluorescence intensity trace; and
(d) outputting a probability of a subject's response to an intervention to reduce the biological effects of space travel as an output of a trained predictive model when the predictive model is provided the subject's exposomic features of spatial fluorescent intensity across a stained one or more biological samples of the subject, wherein the trained predictive model is trained with the first and second set of subjects' exposomic features' one or more metrics, corresponding administered intervention, and clinical response to the administered intervention.

234. The method of claim 233, wherein the trained predictive model is configured to determine whether a biological sample of the subjects has been dysregulated via space travel.

235. The method of claim 233, wherein the trained predictive model is configured to determine the effect of the intervention as a measure of intervention efficacy based at least in part on the analysis of the fluorescence intensity spatially across one or more biological samples.

236. The method of claim 233, wherein the first or second set of one or more biological samples comprise hair, teeth, toenails, fingernails, physiologic parameters, or any combination thereof.

237. The method of claim 233, wherein the intervention comprises a diet, pharmaceutical therapeutic, or any combination thereof.

238. The method of claim 237, wherein the diet comprises iodine supplementation.

239. The method of claim 233, wherein the intervention ameliorates radiation exposure.

240. The method of claim 233, wherein the first or second set of subjects comprise a human, non-human mammal, or any combination thereof.

241. The method of claim 240, wherein the non-human mammal comprises mice.

242. The method of claim 233, wherein the trained predictive model comprises a gradient-boosted ensemble model.

243. The method of claim 233, wherein a measurement of temporal dynamics of the spatial fluorescence intensity traces include: determination of a linear slope, determination of a plurality of non-linear parameters describing curvature of the one or more exposomic signatures, determination of an abrupt change in intensity of the one or more exposomic signatures, determination of one or more changes in a baseline intensity of the one or more exposomic signatures, determination of a change of a frequency-domain representation of the one or more exposomic signatures, determination of a change of the power-spectral domain representation of the one or more exposomic signatures, determination of one or more recurrence quantification analysis parameters, determination of one or more cross-recurrence quantification analysis parameters, determination of one or more joint recurrence quantification analysis parameters, determination of one or more multidimensional recurrence quantification analysis parameters, estimation of a Lyapunov spectra, determination of a maximum Lyapunov exponent, or any combination thereof.

244. The method of claim 233, wherein the measurement of the temporal dynamics comprises determination of one or more the recurrence quantification analysis parameters, wherein the one or more the recurrence quantification analysis parameters comprises a recurrence rate, a determinism, a mean diagonal length, a maximum diagonal length, a divergence, a Shannon entropy in diagonal length, a trend in recurrence, a laminarity, a trapping time, a maximum vertical line length, a Shannon entropy in vertical line length, a mean recurrence time, a Shannon entropy in recurrence time, or a number of a most probable recurrence.

245. The method of claim 233, wherein the analyzing comprises obtaining a fluorescence image of the first and second set stained one or more biological samples and analyzing the spatial fluorescence intensity of the fluorescence image.

246. The method of claim 233, wherein obtaining the fluorescence image of the stained tooth sample comprises using an inverted or non-inverted confocal microscope.

247. The method of any one of claims 233-246, wherein staining the first set of one or more biological samples of a first and second set of subjects comprises using a C-reactive stain.

248. The method of any one of claims 233-246, further comprising sectioning the tooth sample.

249. The method of claim 233, wherein staining the first and second set of one or more biological samples comprises decalcifying the first and second set of one or more biological samples.

250. The method of claim 233, wherein the trained model is selected from the group consisting of: a neural network algorithm, a support vector machine algorithm, a decision tree algorithm, an unsupervised clustering algorithm, a supervised clustering algorithm, a regression algorithm, a gradient-boosting algorithm, and any combination thereof.

251. The method of claim 233, wherein the trained model comprises a gradient-boosted decision tree.

252. The method of claim 233, wherein the trained predictive model comprises a classification algorithm.

253. The method of claim 252, wherein the classification algorithm is trained to identify features in spatial fluorescence intensity associated with the space travel.

254. A method for outputting one or more pathways associated with space travel, comprising:
(a) receiving one or more biological samples of a first set of one or more subjects and a second set of one or more subjects, wherein the first set of one or more subjects has participated in space travel and the second set of one or more subjects has not;
(b) determining one or more pathways' exposomic signatures from the one or more biological samples of the first and second set of one or more subjects;
(c) calculating exposomic features of the one or more pathways' exposomic signatures; and
(d) outputting the one or more pathways associated with space travel by comparing the first and second set of subjects' exposomic features of the one or more pathways.

255. The method of claim 254, wherein each feature of the exposomic features comprises one or more quantitative metrics.

256. The method of claim 254, wherein space travel comprises traveling beyond the Karman line at an altitude of at least 100 kilometers (km) above the Earth's surface.

257. The method of claim 254, wherein the biological sample comprises hair, teeth, toenails, fingernails, physiologic parameters, or any combination thereof.

258. The method of claim 254, wherein the exposomic features comprise a measurement of temporal dynamics of the one or more exposomic signatures.

259. The method of claim 258, wherein the measurement of the temporal dynamics comprises: determination of a linear slope, determination of a plurality of non-linear parameters describing curvature of the one or more exposomic signatures, determination of an abrupt change in intensity of the one or more exposomic signatures, determination of one or more changes in a baseline intensity of the one or more exposomic signatures, determination of a change of a frequency-domain representation of the one or more exposomic signatures, determination of a change of the power-spectral domain representation of the one or more exposomic signatures, determination of one or more recurrence quantification analysis parameters, determination of one or more cross-recurrence quantification analysis parameters, determination of one or more joint recurrence quantification analysis parameters, determination of one or more multidimensional recurrence quantification analysis parameters, estimation of a Lyapunov spectra, determination of a maximum Lyapunov exponent, or any combination thereof.

260. The method of claim 258, wherein the measurement of the temporal dynamics comprises determination of one or more recurrence quantification analysis parameters, wherein the one or more recurrence quantification analysis parameters comprises a recurrence rate, a determinism, a mean diagonal length, a maximum diagonal length, a divergence, a Shannon entropy in diagonal length, a trend in recurrence, a laminarity, a trapping time, a maximum vertical line length, a Shannon entropy in vertical line length, a mean recurrence time, a Shannon entropy in recurrence time, or a number of a most probable recurrence.

261. The method of claim 254, wherein the exposomic signatures are measured by mass spectrometry, laser ablation-inductively coupled plasma-mass spectrometry, laser induced breakdown spectroscopy, Raman spectroscopy, immunohistochemistry fluorescence, or any combination thereof.

262. The method of claim 254, wherein the exposomic features are derived from one or more attractors.

263. The method of claim 260, further comprising analyzing a dynamic relationship between the one or more signal recurrence rates, determinism, mean diagonal length, maximum diagonal length, divergence, Shannon entropy in diagonal length, trend in recurrences, laminarity, trapping time, maximum vertical line length, Shannon entropy in vertical line lengths, mean recurrence time, Shannon entropy in recurrence times, number of the most probable recurrences, or any combination thereof.

264. The method of claim 254, wherein the one or more subjects are a human, non-human mammal, vertebrate, or any combination thereof.

265. A method for providing a treatment to ameliorate the effects of space travel, comprising:
(a) receiving one or more biological samples of one or more subjects participating in space travel;
(b) determining one or more pathways' exposomic signatures from the one or more biological samples of the one or more subjects;
(c) calculating exposomic features of the one or more pathways' exposomic signatures; and
(d) providing a treatment to the one or more subjects, wherein the treatment ameliorates the effect of the space travel on the one or more subjects' exposomic features of the one or more pathways' exposomic signatures.

266. The method of claim 265, wherein the treatment comprises a diet, pharmaceutical, nutraceutical, food bar, or any combination thereof.

267. The method of claim 265, wherein each feature of the exposomic features comprises one or more quantitative metrics.

268. The method of claim 265, wherein the space travel comprises traveling beyond the Karman line at an altitude of at least 100 kilometers (km) above the Earth's surface.

269. The method of claim 265, wherein the biological sample comprises hair, teeth, toenails, fingernails, physiologic parameters, or any combination thereof.

270. The method of claim 265, wherein the exposomic features comprise a measurement of temporal dynamics of the one or more exposomic signatures.

271. The method of claim 270, wherein the measurement of the temporal dynamics comprises: determination of a linear slope, determination of a plurality of non-linear parameters describing curvature of the one or more exposomic signatures, determination of an abrupt change in intensity of the one or more exposomic signatures, determination of one or more changes in a baseline intensity of the one or more exposomic signatures, determination of a change of a frequency-domain representation of the one or more exposomic signatures, determination of a change of the power-spectral domain representation of the one or more exposomic signatures, determination of one or more recurrence quantification analysis parameters, determination of one or more cross-recurrence quantification analysis parameters, determination of one or more joint recurrence quantification analysis parameters, determination of one or more multidimensional recurrence quantification analysis parameters, estimation of a Lyapunov spectra, determination of a maximum Lyapunov exponent, or any combination thereof.

272. The method of claim 270, wherein the measurement of the temporal dynamics comprises determination of one or more recurrence quantification analysis parameters, wherein the one or more recurrence quantification analysis parameters comprises a recurrence rate, a determinism, a mean diagonal length, a maximum diagonal length, a divergence, a Shannon entropy in diagonal length, a trend in recurrence, a laminarity, a trapping time, a maximum vertical line length, a Shannon entropy in vertical line length, a mean recurrence time, a Shannon entropy in recurrence time, or a number of a most probable recurrence.

273. The method of claim 265, wherein the exposomic signatures are measured by mass spectrometry, laser ablation-inductively coupled plasma-mass spectrometry, laser induced breakdown spectroscopy, Raman spectroscopy, immunohistochemistry fluorescence, or any combination thereof.

274. The method of claim 265, wherein the exposomic features are derived from one or more attractors.

275. The method of claim 272, further comprising analyzing a dynamic relationship between the one or more signal recurrence rates, determinism, mean diagonal length, maximum diagonal length, divergence, Shannon entropy in diagonal length, trend in recurrences, laminarity, trapping time, maximum vertical line length, Shannon entropy in vertical line lengths, mean recurrence time, Shannon entropy in recurrence times, number of the most probable recurrences, or any combination thereof.

276. The method of claim 265, wherein the one or more subjects are a human, non-human mammal, vertebrate, or any combination thereof.

277. A method for outputting one or more pathways associated with an intervention, comprising:
(a) receiving one or more biological samples of a first set of one or more subjects and a second set of one or more subjects, wherein the first set of one or more subjects has received an intervention and the second set of one or more subjects has not received an intervention;
(b) determining one or more pathways' exposomic signatures from the one or more biological samples of the first and second set of one or more subjects;
(c) calculating exposomic features of the one or more pathways' exposomic signatures; and
(d) outputting the one or more pathways associated with the intervention by comparing the first and second set of subjects' exposomic features of the one or more pathways.

278. The method of claim 277, wherein each feature of the exposomic features comprises one or more quantitative metrics.

279. The method of claim 277, wherein the biological sample comprises hair, teeth, toenails, fingernails, physiologic parameters, or any combination thereof.

280. The method of claim 277, wherein the exposomic features comprise a measurement of temporal dynamics of the one or more exposomic signatures.

281. The method of claim 280, wherein the measurement of the temporal dynamics comprises: determination of a linear slope, determination of a plurality of non-linear parameters describing curvature of the one or more exposomic signatures, determination of an abrupt change in intensity of the one or more exposomic signatures, determination of one or more changes in a baseline intensity of the one or more exposomic signatures, determination of a change of a frequency-domain representation of the one or more exposomic signatures, determination of a change of the power-spectral domain representation of the one or more exposomic signatures, determination of one or more recurrence quantification analysis parameters, determination of one or more cross-recurrence quantification analysis parameters, determination of one or more joint recurrence quantification analysis parameters, determination of one or more multidimensional recurrence quantification analysis parameters, estimation of a Lyapunov spectra, determination of a maximum Lyapunov exponent, or any combination thereof.

282. The method of claim 280, wherein the measurement of the temporal dynamics comprises determination of one or more recurrence quantification analysis parameters, wherein the one or more recurrence quantification analysis parameters comprises a recurrence rate, a determinism, a mean diagonal length, a maximum diagonal length, a divergence, a Shannon entropy in diagonal length, a trend in recurrence, a laminarity, a trapping time, a maximum vertical line length, a Shannon entropy in vertical line length, a mean recurrence time, a Shannon entropy in recurrence time, or a number of a most probable recurrence.

283. The method of claim 277, wherein the exposomic signatures are measured by mass spectrometry, laser ablation-inductively coupled plasma-mass spectrometry, laser induced breakdown spectroscopy, Raman spectroscopy, immunohistochemistry fluorescence, or any combination thereof.

284. The method of claim 277, wherein the exposomic features are derived from one or more attractors.

285. The method of claim 282, further comprising analyzing a dynamic relationship between the one or more signal recurrence rates, determinism, mean diagonal length, maximum diagonal length, divergence, Shannon entropy in diagonal length, trend in recurrences, laminarity, trapping time, maximum vertical line length, Shannon entropy in vertical line lengths, mean recurrence time, Shannon entropy in recurrence times, number of the most probable recurrences, or any combination thereof.

286. The method of claim 277, wherein the one or more subjects are a human, non-human mammal, vertebrate, or any combination thereof.

287. The method of claim 277, wherein the intervention comprises a pharmaceutical, diet, food bar, nutraceutical, metal ion supplement, or any combination thereof.

288. A method to determine exposomic features of a subject associated with space travel, comprising:
(a) receiving one or more biological samples of a subject;
(b) determining one or more pathways' exposomic signatures from the one or more biological samples;
(c) calculating exposomic features of the one or more pathways' exposomic signatures; and
(d) outputting the exposomic features of the subject associated with space travel by comparing the subject's exposomic features and a database of space travel exposomic features.

289. The method of claim 288, wherein each feature of the exposomic features comprises one or more quantitative metrics.

290. The method of claim 288, wherein the biological sample comprises hair, teeth, toenails, fingernails, physiologic parameters, or any combination thereof.

291. The method of claim 288, wherein the exposomic features comprise a measurement of temporal dynamics of the one or more exposomic signatures.

292. The method of claim 291, wherein the measurement of the temporal dynamics comprises: determination of a linear slope, determination of a plurality of non-linear parameters describing curvature of the one or more exposomic signatures, determination of an abrupt change in intensity of the one or more exposomic signatures, determination of one or more changes in a baseline intensity of the one or more exposomic signatures, determination of a change of a frequency-domain representation of the one or more exposomic signatures, determination of a change of the power-spectral domain representation of the one or more exposomic signatures, determination of one or more recurrence quantification analysis parameters, determination of one or more cross-recurrence quantification analysis parameters, determination of one or more joint recurrence quantification analysis parameters, determination of one or more multidimensional recurrence quantification analysis parameters, estimation of a Lyapunov spectra, determination of a maximum Lyapunov exponent, or any combination thereof.

293. The method of claim 291, wherein the measurement of the temporal dynamics comprises determination of one or more recurrence quantification analysis parameters, wherein the one or more recurrence quantification analysis parameters comprises a recurrence rate, a determinism, a mean diagonal length, a maximum diagonal length, a divergence, a Shannon entropy in diagonal length, a trend in recurrence, a laminarity, a trapping time, a maximum vertical line length, a Shannon entropy in vertical line length, a mean recurrence time, a Shannon entropy in recurrence time, or a number of a most probable recurrence.

294. The method of claim 288, wherein the exposomic signatures are measured by mass spectrometry, laser ablation-inductively coupled plasma-mass spectrometry, laser induced breakdown spectroscopy, Raman spectroscopy, immunohistochemistry fluorescence, or any combination thereof.

295. The method of claim 288, wherein the exposomic features are derived from one or more attractors.

296. The method of claim 293, further comprising analyzing a dynamic relationship between the one or more signal recurrence rates, determinism, mean diagonal length, maximum diagonal length, divergence, Shannon entropy in diagonal length, trend in recurrences, laminarity, trapping time, maximum vertical line length, Shannon entropy in vertical line lengths, mean recurrence time, Shannon entropy in recurrence times, number of the most probable recurrences, or any combination thereof.

297. The method of claim 288, wherein the subject is human, non-human mammal, vertebrate, or any combination thereof.

298. A method to determine exposomic features of a subject associated with space travel with a trained predictive model, comprising:
(a) receiving one or more biological samples of a subject;
(b) determining one or more pathways' exposomic signatures from the one or more biological samples;
(c) calculating exposomic features of the one or more pathways' exposomic signatures; and
(d) outputting the exposomic features of the subject associated with space travel by providing a trained predictive model with the exposomic features of the subject as an input, wherein the trained predictive model is trained on exposomic features of one or more subjects that participated in space travel.

299. The method of claim 298, wherein each feature of the exposomic features comprises one or more quantitative metrics.

300. The method of claim 298, wherein the biological sample comprises hair, teeth, toenails, fingernails, physiologic parameters, or any combination thereof.

301. The method of claim 298, wherein the exposomic features comprise a measurement of temporal dynamics of the one or more exposomic signatures.

302. The method of claim 301, wherein the measurement of the temporal dynamics comprises: determination of a linear slope, determination of a plurality of non-linear parameters describing curvature of the one or more exposomic signatures, determination of an abrupt change in intensity of the one or more exposomic signatures, determination of one or more changes in a baseline intensity of the one or more exposomic signatures, determination of a change of a frequency-domain representation of the one or more exposomic signatures, determination of a change of the power-spectral domain representation of the one or more exposomic signatures, determination of one or more recurrence quantification analysis parameters, determination of one or more cross-recurrence quantification analysis parameters, determination of one or more joint recurrence quantification analysis parameters, determination of one or more multidimensional recurrence quantification analysis parameters, estimation of a Lyapunov spectra, determination of a maximum Lyapunov exponent, or any combination thereof.

303. The method of claim 301, wherein the measurement of the temporal dynamics comprises determination of one or more recurrence quantification analysis parameters, wherein the one or more recurrence quantification analysis parameters comprises a recurrence rate, a determinism, a mean diagonal length, a maximum diagonal length, a divergence, a Shannon entropy in diagonal length, a trend in recurrence, a laminarity, a trapping time, a maximum vertical line length, a Shannon entropy in vertical line length, a mean recurrence time, a Shannon entropy in recurrence time, or a number of a most probable recurrence.

304. The method of claim 298, wherein the exposomic signatures are measured by mass spectrometry, laser ablation-inductively coupled plasma-mass spectrometry, laser induced breakdown spectroscopy, Raman spectroscopy, immunohistochemistry fluorescence, or any combination thereof.

305. The method of claim 298, wherein the exposomic features are derived from one or more attractors.

306. The method of claim 303, further comprising analyzing a dynamic relationship between the one or more signal recurrence rates, determinism, mean diagonal length, maximum diagonal length, divergence, Shannon entropy in diagonal length, trend in recurrences, laminarity, trapping time, maximum vertical line length, Shannon entropy in vertical line lengths, mean recurrence time, Shannon entropy in recurrence times, number of the most probable recurrences, or any combination thereof.

307. The method of claim 298, wherein the subject is human, non-human mammal, vertebrate, or any combination thereof.
